(19) United States
(12) Patent Application Publication
(10) Pub. No.: US 2026/0007674 A1
(43) Pub. Date: Jan. 8, 2026

(54) Novel Chemotypes for Treating Parasitic Diseases and Methods of Use

(71) Applicants: The Medical College of Wisconsin, Inc., Milwaukee, WI (US); University of Florida Research Foundation, Inc., Gainesville, FL (US)

(72) Inventors: Jonathan S. Marchant, Brookfield, WI (US); Daniel J. Sprague, Greenfield, WI (US); Timothy Patrick Spicer, Stuart, FL (US); Louis Scampavia, West Palm Beach, FL (US)

(21) Appl. No.: 18/993,409

(22) PCT Filed: Jul. 13, 2023
(86) PCT No.: PCT/US2023/070148

§ 371 Date: Jan. 10, 2025

Related U.S. Application Data
(60) Provisional application No. 63/388,863, filed on Jul. 13, 2022.
(60) Provisional application No. 63/505,958, filed on Jun. 2, 2023.

(51) Int. Cl. A61K 31/519; A61K 31/166; A61K 31/4162; A61K 31/517; A61P 33/12; C07C 233/69; C07C 233/83; C07D 239/92; C07D 487/04; C07D 495/04

(52) U.S. Cl. CPC A61K 31/519; A61K 31/166; A61K 31/4162; A61K 31/517; A61P 33/12; C07C 233/69; C07C 233/83; C07D 239/92; C07D 487/04; C07D 495/04

(57) ABSTRACT

The present disclosure provides novel chemotypes, pharmaceutical compositions thereof, and method of use thereof for treating parasitic infection, including infection caused by parasitic flatworms. The present compounds may be useful as alternatives to praziquantel and may have improved activities against parasitic infections, particularly parasitic flatworm infections.

### CROSS-REFERENCE TO RELATED APPLICATIONS

The present application claims priority to U.S. Provisional Patent Application No. 63/388,863, filed Jul. 13, 2022 and U.S. Provisional Patent Application No. 63/505,958, filed Jun. 2, 2023, the content of all which are hereby incorporated by reference in its entirety.

### STATEMENT REGARDING FEDERALLY SPONSORED RESEARCH OR DEVELOPMENT

This invention was made with government support under NIAID AI155405 awarded by the National Institutes of Health (NIH). The government has certain rights in the invention.

### BACKGROUND

Infectious diseases caused by parasitic flatworms (e.g. trematodes, cestodes, and monogeneans) cause significant clinical (human) and veterinary (animal) disease worldwide. There are limited drug options for treating these infections which have significant healthcare and economic impact. Given the worldwide burden of neglected tropical diseases, there is ongoing need to develop novel anthelmintic agents to strengthen the pipeline of drugs to combat these burdensome infections. Many diseases caused by parasitic flatworms are treated using the anthelmintic drug praziquantel (PZQ), employed for decades as the key clinical agent to treat schistosomiasis. PZQ activates a flatworm transient receptor potential (TRP) channel within the melastatin family (TRPM_{PZQ}) to mediate sustained Ca²⁺ influx and worm paralysis.

There is a need to develop novel anthelmintic agents to help battle parasitic infections, which is still a worldwide problem. Alternatives to PZQ are needed. PZQ has several features that could be improved and the threat of drug resistance, potentially accelerated by the rollout of recent mass drug administration (MDA) initiatives, persists.

### SUMMARY OF THE INVENTION

The present disclosure describes novel chemotypes that are broad-spectrum flatworm TRPM_{PZQ} (transient receptor potential melastatin-praziquantel) channel activators, and methods of use thereof for treatment of parasitic infections, particularly parasitic flatworm infections.

In one aspect, the present disclosure provides compound of Formula (I′), or a pharmaceutically acceptable salt thereof:

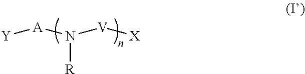

wherein
A is a ring system selected from the group consisting of

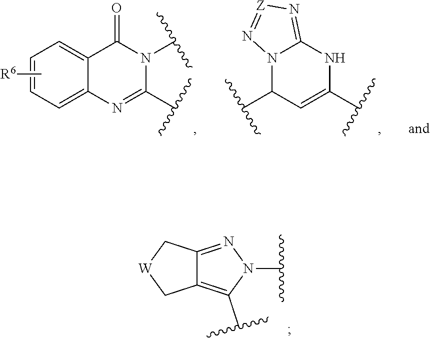

or A is an alkenyl of

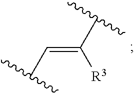

X is alkyl, aryl, heteroaryl, C₁₋₄ alkylenearyl, or cycloalkyl, wherein X is optionally substituted with one or more substituents selected from the group consisting of alkyl, haloalkyl, alkoxy, halogen, cyano, deuterium, and nitro;
Y is alkyl, cycloalkyl, aryl, C₁₋₄ alkylenearyl, or heteroaryl, wherein Y is optionally substituted with one or more substituents selected from the group consisting of halogen, alkoxy optionally substituted with one or more halogen, alkyl, haloalkyl, and nitro;
Z is N or CR⁵;
W is S, S(O), or S(O)₂;
V is C(O) or S(O)₂;
n is 0 or 1; and
   wherein
R is hydrogen, alkyl, aryl, cycloalkyl, or heteroaryl;
R¹ is hydrogen or alkyl;
R² is hydrogen, alkyl optionally substituted with —C(O)OR¹, or aryl optionally substituted with alkyl;
R³ is —OC(O)R¹, —C(O)OR¹, or —C(O)NR²R⁴;
R⁴ is alkyl optionally substituted with hydroxy;
R⁵ is selected from the group consisting of hydrogen, hydroxyalkyl, —OC(O)R¹, —C(O)OR¹, —NR¹C(O)R², and —NR¹S(O)₂R²; and
R⁶ is an optional substituent selected from the group consisting of halogen, alkyl, haloalkyl, alkoxy, cyano, and nitro,
provided that the compound is not 4-bromo-N-(2-(4-fluorophenyl)-4-oxoquinazolin-3(4H)-yl)benzamide or 4-bromo-N-(2-(2-fluorophenyl)-4-oxoquinazolin-3(4H)-yl)benzamide.

In some embodiments, the compound of Formula (I′), or a pharmaceutically acceptable salt thereof, has a structure of Formula I(a):

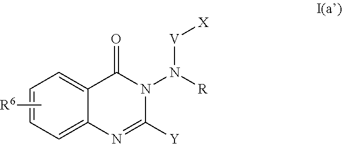

wherein
X is alkyl, aryl, heteroaryl, C₁₋₄ alkylenearyl, or cycloalkyl, wherein X is optionally substituted with one or more substituents selected from the group consisting of alkyl, haloalkyl, alkoxy, halogen, cyano, deuterium, and nitro;
Y is alkyl, cycloalkyl, aryl, C₁₋₄ alkylenearyl, or heteroaryl, wherein Y is optionally substituted with one or more substituents selected from the group consisting of halogen, alkoxy optionally substituted with one or more halogen, alkyl, haloalkyl, and nitro;
R is hydrogen, alkyl, aryl, cycloalkyl, or heteroaryl; and
R⁶ is an optional substituent selected from the group consisting of halogen and alkyl.

In another aspect, the present disclosure provides a composition comprising the compound as described herein, or a pharmaceutically acceptable salt thereof, and a pharmaceutically acceptable carrier.

In another aspect, the present disclosure provides a method of treating a parasitic infection in a subject in need thereof, the method comprises administering to the subject an effective amount of the compound as described herein, or a pharmaceutically acceptable salt thereof, or the composition as described herein. In some embodiments, the parasitic infection is a parasitic flatworm infection. For example, the parasitic flatworm can be trematode, cestode, monogenean, schistosome, tapeworm, or a combination thereof.

In another aspect, the present disclosure provides a method of treating schistosomiasis in a subject in need thereof, the method comprises administering to the subject an effective amount of the compound as described herein, or a pharmaceutically acceptable salt thereof, or the composition as described herein.

In another aspect, the present disclosure provides a method of activating a transient receptor potential channel of a parasitic flatworm, the method comprising introducing to the parasitic flatworm an effective amount of the compound as described herein, or a salt thereof, or the composition as described herein.

In another aspect, the present disclosure provides a method of causing paralysis or inhibiting growth of a parasitic flatworm, the method comprising introducing to the parasitic flatworm an effective amount of the compound as described herein, or a salt thereof, or the composition as described herein.

In some embodiments, the parasitic flatworm is in an infected subject. In some embodiments, the subject is a human.

### BRIEF DESCRIPTION OF THE DRAWINGS

FIG. 1 shows the structure of screened compounds 1-9 that are N-amidoquinazolinones.

FIG. 2 shows dose-response curves of compounds 1-9. Representative data from SmTRP_{PZQ} activation (▪), FhTRP_{PZQ} activation (▴), and HEK activation (negative control, •) are shown.

FIG. 3 shows that N-amidoquinazolinones contract S. mansoni in a manner similar to the channel activator (and standard of treatment) praziquantel.

FIG. 4 shows the structures of screened compounds 10-12 and their dose-response curves. Representative data from SmTRP_{PZQ} activation (▪), FhTRP_{PZQ} activation (▴), and HEK activation (negative control, •) are shown.

FIG. 5 shows the structures of screened compounds 13-15 and their dose-response curves. Representative data from SmTRP_{PZQ} activation (▪), FhTRP_{PZQ} activation (▴), and HEK activation (negative control, •) are shown.

FIG. 6 shows the structures of screened compounds 16-18 and their dose-response curves. Representative data from SmTRP_{PZQ} activation (▪), FhTRP_{PZQ} activation (▴), and HEK activation (negative control, •) are shown.

FIG. 7 shows the structures of screened compounds 19-21 and their dose-response curves. Representative data from SmTRP_{PZQ} activation (▪), FhTRP_{PZQ} activation (▴), and HEK activation (negative control, •) are shown.

FIG. 8 shows the structures of screened compounds 22-24 and their dose-response curves. Representative data from SmTRP_{PZQ} activation (▪). FhTRP_{PZQ} activation (▴), and HEK activation (negative control, •) are shown.

FIG. 9 shows the structures of screened compounds 25-27 and their dose-response curves. Representative data from SmTRP_{PZQ} activation (▪), FhTRP_{PZQ} activation (▴), and HEK activation (negative control, •) are shown.

FIG. 10 shows the structures of screened compounds 28-30 and their dose-response curves. Representative data from SmTRP_{PZQ} activation (▪), FhTRP_{PZQ} activation (▴), and HEK activation (negative control, •) are shown.

FIG. 11 shows the structures of screened compound 31 and its dose-response curve. Representative data from SmTRP_{PZQ} activation (▪), FhTRP_{PZQ} activation (▴), and HEK activation (negative control, •) are shown.

FIG. 12 shows the structures of screened compounds 32-34 and their dose-response curves. Representative data from SmTRP_{PZQ} activation (▪). FhTRP_{PZQ} activation (▴), and HEK activation (negative control, •) are shown.

FIG. 13 shows that 2-amidoacrylates or 2-amidoacrylamides contract S. mansoni in a manner similar to the channel activator (and standard of treatment) praziquantel.

FIG. 14 shows the structures of screened compounds 40-41 and their dose-response curves. Representative data from SmTRP_{PZQ} activation (▪), FhTRP_{PZQ} activation (▴), and HEK activation (negative control, •) are shown.

FIGS. 15A-C show the putative binding pose of Praziquantel and BZQ in Sm.TRPM_{PZQ} and Fh.TRPM_{PZQ}.

FIG. 16 shows representative results of in vivo treatment of schistosomiasis using BZQ/S55.

FIGS. 17A-L show representative BZQ/S55 ex vivo activities on Fasciola hepatica adults and immature worms. FIG. 17A shows Schistosomes treated with DMSO (control) or Praziquantel (500 nM). Praziquantel causes a rapid, spastic paralysis, but DMSO treatment has no effect. FIG. 17B shows Fasciola hepatica treated with DMSO, or praziquantel (50 μM). Neither treatment has an effect on the liver fluke. FIG. 17C shows Schistosomes and Fasciola hepatica treated with BZQ/S55. Rapid, sustained contraction and paralysis in both species were observed. FIG. 17D shows the motility scoring of immature F. hepatica treated with various drugs. FIG. 17E shows the motility scoring of immature, triclabendazole-resistant F. hepatica treated with various drugs. FIG. 17F shows the motility scoring of adult F. hepatica treated with various drugs. FIG. 17G shows the electron microscopy image of S. mansoni treated with DMSO (tegument appeared intact). FIG. 17H shows the electron microscopy image of S. mansoni treated with praziquantel (tegument was destroyed). FIG. 17I shows the electron microscopy image of S. mansoni treated with BZQ/S55 (tegument was destroyed). FIG. 17J shows the electron microscopy image of F. hepatica treated with DMSO (no effect). FIGS. 17K and 17L show the electron microscopy images of F. hepatica treated with BZQ/S55 (notable tegument damage was observed, consistent with activation of TRPM_{PZQ} in other species).

The drawings illustrate only example embodiments and are therefore not to be considered limiting of the scope of the embodiments described herein, as other embodiments are within the scope of the disclosure.

### DETAILED DESCRIPTION OF THE INVENTION′

Before the present materials and methods are described, it is understood that this invention is not limited to the particular methodology, protocols, materials, and reagents described, as these may vary. It is also to be understood that the terminology used herein is for the purpose of describing particular embodiments only and is not intended to limit the scope of the present invention which will be limited only by the appended claims.

As used in this specification and the claims, the singular forms “a,” “an,” and “the” include plural forms unless the context clearly dictates otherwise. For example, the term “a compound” should be interpreted to mean “one or more compounds” unless the context clearly dictates otherwise. As used herein, the term “plurality” means “two or more.”

As used herein, the terms “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

Unless defined otherwise, all technical and scientific terms used herein have the same meanings as commonly understood by one of ordinary skill in the art to which this disclosure pertains. All publications and patents specifically mentioned herein are incorporated by reference for all purposes, including those describing and disclosing the chemicals, cell lines, vectors, animals, instruments, statistical analysis and methodologies which might be useful in connection with the present disclosure. In the case of conflict, the present specification, including definitions, will control.

### Definitions

Chemical entities and the use thereof may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a saturated straight or branched divalent hydrocarbon group having 1-12, 1-10, or 1-6 carbon atoms, referred to herein as C₁-C₁₂ alkylene, C₁-C₁₀-alkylene, and C₁-C₆-alkylene, respectively. An exemplary alkylene group is —CH₂CH₂—.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy. In some instances, the number of carbon atoms in an alkoxy moiety is indicated by the prefix “C_{x-y}”, wherein x is the minimum and y is the maximum number of carbon atoms in the substituent. Thus, for example, “C₁₋₆ alkoxy” means an alkoxy substituent containing from 1 to 6 carbon atoms and “C₁₋₄ alkoxy” means an alkoxy substituent containing from 1 to 4 carbon atoms.

The term “carboxy” or “carboxyl” as used herein refers to the group-COOH or its corresponding salts, e.g. —COONa, etc.

The term “aryl” is art-recognized and refers to a carbocyclic aromatic group. Representative aryl groups include phenyl, naphthyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. The term “phenyl” refers to a mono-substituted benzene ring and has a formula of —C₆H₅.

The term “heteroaryl” is art-recognized and refers to a heterocyclic aromatic group. Representative heteroaryl groups include pyridinyl, quinolinyl, furanyl, thionyl, and the like. The term “heteroaryl” includes polycyclic ring systems having two or more heterocyclic rings in which two or more carbon or heteroatom are common to two adjoining rings (the rings are “fused rings”) wherein at least one of the rings is a heterocyclic aromatic group and, e.g., the other ring(s) may be cycloalkyls, cycloalkenyls, cycloalkynyls, and/or aryls. In certain embodiments, the heteroaryl group is a 6-10 membered ring structure. The term “pyridyl” refers to a group derived from pyridine by removal of a hydrogen atom from a ring carbon atom in pyridine. The pyridyl group has a formula —C₅H₄N.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The terms “heterocycloalkyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C3-C7” indicates that the heterocyclic ring contains a total of from 3 to 7 ring atoms, inclusive of any heteroatoms that occupy a ring atom position. In one embodiment, the heterocyclyl is piperidinyl.

The term “halogen” refers to halogen atoms F, Cl, Br, and I, or halogen substituents fluoro (—F), chloro (—Cl), bromo (—Br), and iodo- (—I).

The term “haloalkyl” is art-recognized and refers to an alkyl group, as defined above, having halogen atoms, as defined above, replacing one or more hydrogen atoms. Representative haloalkyl groups include trifluoromethyl, dibromoethyl, monochloropropyl, and the like.

The term “hydroxy” refers to a group of the form-OH.

The term “hydroxyalkyl” refers to an alkyl means an alkyl, as defined herein, in which a hydrogen atom is replaced by —OH. Representative examples of hydroxyalkyl include, but are not limited to those derived from C₁₋₆ alkyls, such as —CH₂OH, —CH₂CH₂OH, —CH₂CH₂CH₂OH, and the like.

The term “nitro” refers to a group of the form —NO₂.

The term “thiophenyl” refers to a group of the form

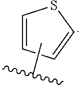

The term “1,3-benzodioxolyl” refers to a group of the form

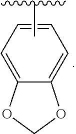

The term “cyano” refers to a group of the form-CN.

As used herein, the dashed line ( [symbol] ) in the formulae as described herein is an optional single bond. The symbol having one dashed line on top of a solid line (“ [symbol] ”) refers to a single bond that may optionally be a double bond.

Terms such as “alkyl,” “cycloalkyl,” “alkylene,” “cycloalkylene,” “polycycloalkylidene” etc. may be preceded by a designation indicating the number of atoms present in the group in a particular instance (e.g., “C₁-C₄alkyl,” “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁-C₄” or “C₁₋₄,” the members of the group that follows may have any number of carbon atoms falling within the recited range. A “C₁-C₄alkyl” or “C₁₋₄alkyl,” for example, is an alkyl group having from 1 to 4 carbon atoms, however arranged (i.e., straight chain or branched).

If a group is described as being “substituted”, a non-hydrogen substituent group is in the place of hydrogen on a carbon or nitrogen of that group. Thus, for example, a substituted alkyl is an alkyl in which at least one non-hydrogen group is in the place of a hydrogen on the alkyl. To illustrate, monofluoroalkyl is alkyl substituted with a fluoro group, and difluoroalkyl is alkyl substituted with two fluoro groups. It should be recognized that if there is more than one substitution on a substituent, each non-hydrogen group may be identical or different (unless otherwise stated). Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

When a group is referred to as “unsubstituted” or not referred to as “substituted” or “optionally substituted”, it means that the group does not have any substituents. If a group is described as being “optionally substituted”, the group may be either (1) not substituted or (2) substituted. If a group is described as being optionally substituted with up to a particular number of non-hydrogen substituents, that group may be either (1) not substituted; or (2) substituted by up to that particular number of substituent groups or by up to the maximum number of substitutable positions on that group, whichever is less.

If substituents are described as being independently selected from a group, each substituent is selected independent of the other. Each substituent, therefore, may be identical to or different from the other substituent(s).

A person of ordinary skill in the art would be able to choose the substituents that fulfill the valency rules. For example, in a non-solvated or non-salt form of a compound, nitrogen typically has three bonds attached to it and oxygen typically has two bonds attached to it.

As used herein. “salt” refers to acid addition salts and basic addition salts. It may also refer to those salts that may be prepared in situ during the final isolation and purification of the present compounds.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Pharmaceutically acceptable salts of the present compounds are contemplated and may be utilized in the methods disclosed herein. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-, 1,4-dioate, hexyne-1,6-dioate, benzoate, chlorobenzoate, methylbenzoate, hydroxy benzoate, methoxy benzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

### Compounds

In one aspect, the present disclosure provides a compound of Formula (I′), or a pharmaceutically acceptable salt thereof,

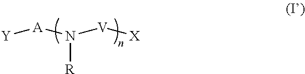

wherein
   A is a ring system selected from the group consisting of

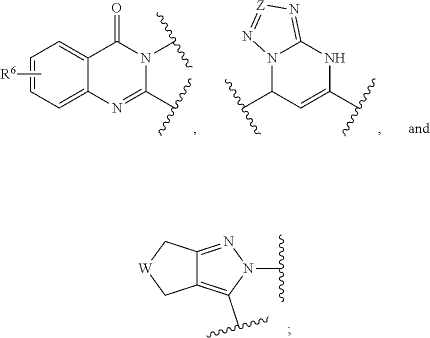

or A is an alkenyl of

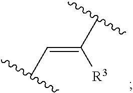

X is alkyl, aryl, heteroaryl, C₁₋₄ alkylenearyl, or cycloalkyl, wherein X is optionally substituted with one or more substituents selected from the group consisting of alkyl, haloalkyl, alkoxy, halogen, cyano, deuterium, and nitro;
Y is alkyl, cycloalkyl, aryl, C₁₋₄ alkylenearyl, or heteroaryl, wherein Y is optionally substituted with one or more substituents selected from the group consisting of halogen, alkoxy optionally substituted with one or more halogen, alkyl, haloalkyl, and nitro;
Z is N or CR⁵;
W is S, S(O), or S(O)₂;
V is C(O) or S(O)₂;
n is 0 or 1; and
wherein
   R is hydrogen, alkyl, aryl, cycloalkyl, or heteroaryl;
   R¹ is hydrogen or alkyl;
   R² is hydrogen, alkyl optionally substituted with —C(O)OR¹, or aryl optionally substituted with alkyl;
   R³ is —OC(O)R¹, —C(O)OR′, or —C(O)NR²R⁴;
   R⁴ is alkyl optionally substituted with hydroxy;
   R⁵ is selected from the group consisting of hydrogen, hydroxyalkyl, —OC(O)R′, —C(O)OR¹, —NR¹C(O)R², and —NR'S(O)₂R²; and
   R⁶ is an optional substituent selected from the group consisting of halogen, alkyl, haloalkyl, alkoxy, cyano, and nitro,
   provided that the compound is not 4-bromo-N-(2-(4-fluorophenyl)-4-oxoquinazolin-3(4H)-yl)benzamide or 4-bromo-N-(2-(2-fluorophenyl)-4-oxoquinazolin-3(4H)-yl)benzamide.

In some embodiments, V is C(O). In some embodiments, V is S(O)₂.

In some embodiments, R⁶ is absent. In some embodiments, R⁶ is halogen (e.g., Cl). In some embodiments, R⁶ is alkyl (e.g., methyl).

In some embodiments, the compound of Formula (I′), or a pharmaceutically acceptable salt thereof, has a structure of Formula (I),

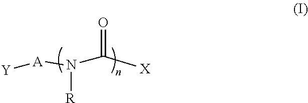

wherein
   A is a ring system selected from the group consisting of

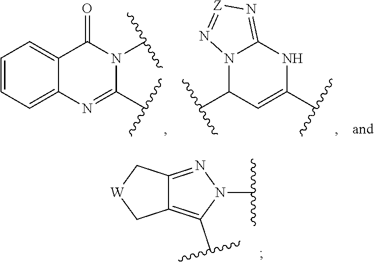

or A is an alkenyl of

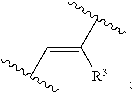

X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
Z is N or CR⁵;
W is S, S(O), or S(O)₂;
n is 0 or 1; and
wherein
   R is hydrogen, alkyl, aryl, cycloalkyl, or heteroaryl;
   R¹ is hydrogen or alkyl;
   R² is hydrogen, alkyl optionally substituted with —C(O)OR¹, or aryl optionally substituted with alkyl;
   R³ is —OC(O)R¹, —C(O)OR¹, or —C(O)NR²R⁴;
   R⁴ is alkyl optionally substituted with hydroxy; and
   R⁵ is selected from the group consisting of hydrogen, hydroxyalkyl, —OC(O)R¹, —C(O)OR¹, —NR¹C(O)R², and —NR¹S(O)₂R².

In some embodiments, the compound of Formula (I′), or a pharmaceutically acceptable salt thereof, has a structure of Formula I(a′),

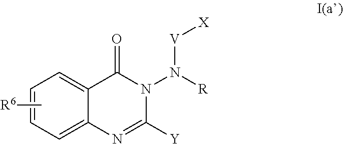

wherein
   X is alkyl, aryl, heteroaryl, C₁₋₄ alkylenearyl, or cycloalkyl, wherein X is optionally substituted with one or more substituents selected from the group consisting of alkyl, haloalkyl, alkoxy, halogen, cyano, deuterium, and nitro;
   Y is alkyl, cycloalkyl, aryl, C₁₋₄ alkylenearyl, or heteroaryl, wherein Y is optionally substituted with one or more substituents selected from the group consisting of halogen, alkoxy optionally substituted with one or more halogen, alkyl, haloalkyl, and nitro;
   R is hydrogen, alkyl, aryl, cycloalkyl, or heteroaryl; and
   R⁶ is an optional substituent selected from the group consisting of halogen and alkyl.

In some embodiments, V in Formula I(a) is C(O). In some embodiments, V in formula I(a′) is S(O)₂.

In some embodiments, R⁶ in Formula I(a′) is absent. In some embodiments, R⁶ in Formula I(a′) is halogen (e.g., Cl). In some embodiments, R⁶ in Formula I(a′) is alkyl (e.g., methyl).

In some embodiments, the compound of Formula (I), or a pharmaceutically acceptable salt thereof, has a structure of Formula I(a),

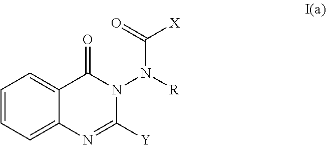

wherein
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro; and
R is hydrogen, alkyl, aryl, cycloalkyl, or heteroaryl.

In some embodiments, X in compounds of Formula (I′), Formula (I), Formula I(a′), or Formula I(a) is alkyl, cycloalkyl, phenyl, naphthyl, furanyl, pyridazyl, benzyl, pyridyl, or thionyl, wherein the cycloalkyl, phenyl, naphthyl, furanyl, pyridazyl, benzyl, pyridyl, or thionyl is optionally substituted with one or more substituents selected from the group consisting of alkoxy, alkyl, haloalkyl, cyano, deuterium, and halogen. For example, X can be an optionally substituted cycloalkyl, an optionally substituted phenyl, an optionally substituted an optionally substituted naphthyl, an optionally substituted furanyl, an optionally substituted pyridazyl, an optionally substituted benzyl, an optionally substituted pyridyl, or an optionally substituted thionyl.

In some embodiments, X is pyridyl, or phenyl optionally substituted with one or more substituents selected from the group consisting of alkoxy, alkyl, and halogen.

In some embodiments, X is phenyl substituted with 1, 2, 3, 4 or 5 deuterium.

In some embodiments, X is phenyl, phenyl mono-substituted at 2- or 3-position, or phenyl bi-substituted at both 2- and 3-position. The numbering of substituents begins with the carbon atom on the phenyl ring attached to the remainder of the molecule, as follows:

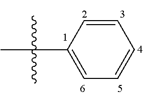 [SMILES: *c1ccccc1]

In some embodiments, Y in compounds of Formula (I), Formula (I′), Formula I(a), or Formula I(a) is alkyl, cycloalkyl, furanyl, benzyl, thionyl, or phenyl, wherein the cycloalkyl, furanyl, benzyl, thionyl, or phenyl is optionally substituted with one or more groups selected from the group consisting of halogen, haloalkyl, alkoxy optionally substituted with one or more halogen, and nitro.

In some embodiments, Y is optionally substituted phenyl. In some embodiments, Y is phenyl optionally substituted with one or more groups selected from the group consisting of halogen and nitro. In some embodiments, Y is 3-chlorophenyl.

In some embodiments of compounds of Formula (I′), Formula (I), Formula I(a′), or Formula I(a). X is alkyl, cycloalkyl, phenyl, naphthyl, furanyl, pyridazyl, benzyl, pyridyl, or thionyl, wherein the cycloalkyl, phenyl, naphthyl, furanyl, pyridazyl, benzyl, pyridyl, or thionyl is optionally substituted with one or more substituents selected from the group consisting of alkoxy, alkyl, haloalkyl, cyano, deuterium, and halogen; and Y is alkyl, cycloalkyl, furanyl, benzyl, thionyl, or phenyl, wherein the cycloalkyl, furanyl, benzyl, thionyl, or phenyl is optionally substituted with one or more groups selected from the group consisting of halogen, haloalkyl, alkoxy optionally substituted with one or more halogen, and nitro.

In some embodiments of compounds of Formula (I′), Formula (I), Formula I(a′), or Formula I(a), X is pyridyl, or phenyl optionally substituted with one or more substituents selected from the group consisting of alkoxy, alkyl, and halogen; and Y is phenyl optionally substituted with one or more groups selected from the group consisting of halogen and nitro.

In some embodiments, the compound of Formula (I), Formula (I′), Formula I(a), or Formula I(a′) is selected from the group consisting of:
2-methoxy-N-(2-(3-nitrophenyl)-4-oxoquinazolin-3(4H)-yl)benzamide.
N-(2-(3-chlorophenyl)-4-oxoquinazolin-3(4H)-yl)-2-methoxybenzamide;
N-(2-(3-chlorophenyl)-4-oxoquinazolin-3(4H)-yl)-2-methylbenzamide;
N-(2-(3-chlorophenyl)-4-oxoquinazolin-3(4H)-yl)-2-fluorobenzamide;
N-(2-(3-chlorophenyl)-4-oxoquinazolin-3(4H)-yl) isonicotinamide;
3-chloro-N-(2-(3-chlorophenyl)-4-oxoquinazolin-3(4H)-yl)benzamide;
N-(2-(3-chlorophenyl)-4-oxoquinazolin-3(4H)-yl)-4-fluorobenzamide;
5-bromo-2-chloro-N-(2-(3-chlorophenyl)-4-oxoquinazolin-3(4H)-yl)benzamide;
2-bromo-N-(2-(3-chlorophenyl)-4-oxoquinazolin-3(4H)-yl)-5-methoxybenzamide;
N-(2-(3-chlorophenyl)-4-oxoquinazolin-3(4H)-yl)benzamide;
N-(2-(3-chlorophenyl)-4-oxoquinazolin-3(4H)-yl)benzamide-2,3,4,5,6-d₅;
N-(2-(3-chlorophenyl)-4-oxoquinazolin-3(4H)-yl)-2-(trifluoromethyl)benzamide;
N-(2-(3-chlorophenyl)-4-oxoquinazolin-3(4H)-yl)-2-nitrobenzamide;
2-chloro-N-(2-(3-chlorophenyl)-4-oxoquinazolin-3(4H)-yl)benzamide;
N-(2-(3-chlorophenyl)-4-oxoquinazolin-3(4H)-yl)-3-fluorobenzamide;
N-(2-(3-chlorophenyl)-4-oxoquinazolin-3(4H)-yl)-3-nitrobenzamide;
N-(2-(3-chlorophenyl)-4-oxoquinazolin-3(4H)-yl)-3-methylbenzamide;
N-(2-(3-chlorophenyl)-4-oxoquinazolin-3(4H)-yl)-3-methoxybenzamide;
N-(2-(3-chlorophenyl)-4-oxoquinazolin-3(4H)-yl)-2,6-difluorobenzamide;
N-(2-(3-chlorophenyl)-4-oxoquinazolin-3(4H)-yl)-4-methylbenzamide;
N-(2-(3-chlorophenyl)-4-oxoquinazolin-3(4H)-yl)-4-nitrobenzamide;
N-(2-(3-chlorophenyl)-4-oxoquinazolin-3(4H)-yl)-4-methoxybenzamide;
N-(2-(3-chlorophenyl)-4-oxoquinazolin-3(4H)-yl)-2-naphthamide;
N-(2-(3-chlorophenyl)-4-oxoquinazolin-3(4H)-yl) furan-2-carboxamide;
N-(2-(3-chlorophenyl)-4-oxoquinazolin-3(4H)-yl)-1-naphthamide;
N-(2-(3-chlorophenyl)-4-oxoquinazolin-3(4H)-yl)-2-fluoronicotinamide;
N-(2-(3-chlorophenyl)-4-oxoquinazolin-3(4H)-yl)thiophene-2-carboxamide;
N-(2-(3-chlorophenyl)-4-oxoquinazolin-3(4H)-yl)thiophene-3-carboxamide;
N-(2-(3-chlorophenyl)-4-oxoquinazolin-3(4H)-yl)cyclohexanecarboxamide;
N-(2-(3-chlorophenyl)-4-oxoquinazolin-3(4H)-yl)-2-phenylacetamide;
N-(2-(3-chlorophenyl)-4-oxoquinazolin-3(4H)-yl) acetamide;
N-(2-(3-chlorophenyl)-4-oxoquinazolin-3(4H)-yl)-N-methylbenzamide;
N-(4-oxo-2-(2-(trifluoromethyl)phenyl)quinazolin-3(4H)-yl)benzamide;
N-(2-(3-methoxyphenyl)-4-oxoquinazolin-3(4H)-yl)benzamide;
N-(4-oxo-2-(m-tolyl)quinazolin-3(4H)-yl)benzamide;
N-(2-(3-nitrophenyl)-4-oxoquinazolin-3(4H)-yl)benzamide;
N-(4-oxo-2-(3-(trifluoromethoxy)phenyl)quinazolin-3(4H)-yl)benzamide;
N-(4-oxo-2-(3-(trifluoromethyl)phenyl)quinazolin-3(4H)-yl)benzamide;
N-(2-(4-methoxyphenyl)-4-oxoquinazolin-3(4H)-yl)benzamide;
N-(2-(4-chlorophenyl)-4-oxoquinazolin-3(4H)-yl)benzamide;
2-fluoro-N-(2-methyl-4-oxoquinazolin-3(4H)-yl)benzamide;
N-(4-oxo-2-phenylquinazolin-3(4H)-yl)benzamide;
N-(2-cyclohexyl-4-oxoquinazolin-3(4H)-yl)benzamide;
N-(4-oxo-2-(thiophen-2-yl)quinazolin-3(4H)-yl)benzamide;
N-(4-oxo-2-(thiophen-3-yl)quinazolin-3(4H)-yl)benzamide;
N-(2-(furan-2-yl)-4-oxoquinazolin-3(4H)-yl)benzamide;
N-(2-(3-chlorobenzyl)-4-oxoquinazolin-3(4H)-yl)benzamide;
N-(2-(3-chlorophenyl)-5-methyl-4-oxoquinazolin-3(4H)-yl)benzamide;
N-(5-chloro-2-(3-chlorophenyl)-4-oxoquinazolin-3(4H)-yl)benzamide;
N-(2-(3-chlorophenyl)-6-methyl-4-oxoquinazolin-3(4H)-yl)benzamide;
N-(6-chloro-2-(3-chlorophenyl)-4-oxoquinazolin-3(4H)-yl)benzamide;
N-(7-chloro-2-(3-chlorophenyl)-4-oxoquinazolin-3(4H)-yl)benzamide;
N-(2-(3-chlorophenyl)-8-methyl-4-oxoquinazolin-3(4H)-yl)benzamide;
N-(8-chloro-2-(3-chlorophenyl)-4-oxoquinazolin-3(4H)-yl)benzamide;
3-methyl-N-(4-oxo-2-(thiophen-2-yl)quinazolin-3(4H)-yl)benzamide;
2-methoxy-N-(2-(3-nitrophenyl)-4-oxoquinazolin-3(4H)-yl)benzamide;
N-(2-(5-chlorothiophen-3-yl)-4-oxoquinazolin-3(4H)-yl)-2-fluorobenzamide;
N-(2-(3-chlorophenyl)-4-oxoquinazolin-3(4H)-yl) furan-2-carboxamide;
N-(2-(3-chlorophenyl)-4-oxoquinazolin-3(4H)-yl)-2-nitrobenzamide;
2-fluoro-N-(4-oxo-2-phenylquinazolin-3(4H)-yl)benzamide;
N-(2-cyclohexyl-4-oxoquinazolin-3(4H)-yl)-2-fluorobenzamide;
N-(2-(3-chlorophenyl)-4-oxoquinazolin-3(4H)-yl)-3-cyanobenzamide;
N-(2-(3-chlorophenyl)-4-oxoquinazolin-3(4H)-yl)-5-fluorothiophene-2-carboxamide;
N-(2-(3-chlorophenyl)-4-oxoquinazolin-3(4H)-yl)-4-cyanobenzamide;
N-(2-(3-chlorophenyl)-4-oxoquinazolin-3(4H)-yl) nicotinamide;
N-(2-(3-chlorophenyl)-4-oxoquinazolin-3(4H)-yl) picolinamide;
N-(2-(3-chlorophenyl)-4-oxoquinazolin-3(4H)-yl)cyclohexanesulfonamide;
N-(2-(3-chlorophenyl)-6-methyl-4-oxoquinazolin-3(4H)-yl)-2-fluorobenzamide;
N-(2-(3-chlorophenyl)-4-oxoquinazolin-3(4H)-yl)pyridazine-3-carboxamide;
2-fluoro-N-(2-methyl-4-oxoquinazolin-3(4H)-yl)benzamide;
N-(6-chloro-2-(3-chlorophenyl)-4-oxoquinazolin-3(4H)-yl)-2-fluorobenzamide;
2-bromo-N-(2-(3-chlorophenyl)-4-oxoquinazolin-3(4H)-yl)benzamide;
N-(2-(5-chlorothiophen-3-yl)-4-oxoquinazolin-3(4H)-yl)-2-fluorobenzamide;
N-(2-(3-chlorophenyl)-4-oxoquinazolin-3(4H)-yl)-5-fluorothiophene-2-carboxamide;
4-chloro-N-(2-(3-chlorophenyl)-4-oxoquinazolin-3(4H)-yl)benzenesulfonamide; and
N-(2-(3-chlorophenyl)-4-oxoquinazolin-3(4H)-yl)-2-fluorobenzenesulfonamide, or pharmaceutically acceptable salt thereof.

In some embodiments, the compound of Formula (I), Formula (I′), Formula I(a), or
Formula I(a) is
N-(2-(3-chlorophenyl)-4-oxoquinazolin-3(4H)-yl)benzamide; or
N-(2-(3-chlorophenyl)-4-oxoquinazolin-3(4H)-yl)benzamide-2,3,4,5,6-d5,
or a pharmaceutically acceptable salt thereof.

In some other embodiments, the compound of Formula (I′) or Formula (I), or a pharmaceutically acceptable salt thereof, has a structure of Formula I (b),

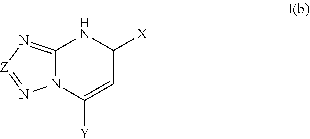

wherein
Z is N or CR⁵;
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
R⁵ is hydrogen, hydroxyalkyl, —OC(O)R¹, —C(O)OR¹, —NR¹C(O)R², or —NR¹S(O)₂R²;
R¹ is hydrogen or alkyl; and
R² is hydrogen, alkyl optionally substituted with —C(O)OR¹, or aryl optionally substituted with alkyl.

In some embodiments, X in compounds of Formula I (b) is phenyl optionally substituted with one or more substituents selected from the group consisting of halogen, alkoxy, and alkyl, or X is thiophenyl.

In some embodiments, Y in compounds of Formula I (b) is phenyl or thiophenyl, that is optionally substituted with one or more substituents selected from the group consisting of alkyl, halogen, alkoxy, and alkoxy substituted with one or more halogen, or Y is 1,3-benzodioxolyl.

In some embodiments of compounds of Formula I (b), X is phenyl optionally substituted with one or more substituents selected from the group consisting of halogen, alkoxy, and alkyl, or X is thiophenyl, and Y is phenyl or thiophenyl, that is optionally substituted with one or more substituents selected from the group consisting of alkyl, halogen, alkoxy, and alkoxy substituted with one or more halogen, or Y is 1,3-benzodioxolyl.

In some embodiments, the compound of Formula I (b) is

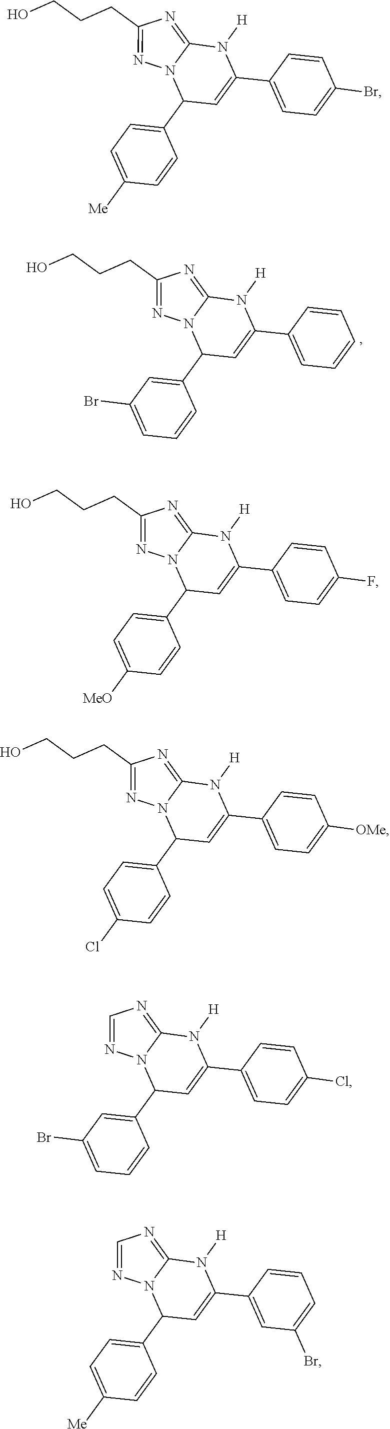 [SMILES: Cc1ccc(C2C=C(c3ccc(Br)cc3)Nc3nc(CCCO)nn32)cc1 ; OCCCc1nc2n(n1)C(c1cccc(Br)c1)C=C(c1ccccc1)N2 ; COc1ccc(C2C=C(c3ccc(F)cc3)Nc3nc(CCCO)nn32)cc1 ; COc1ccc(C2=CC(c3ccc(Cl)cc3)n3nc(CCCO)nc3N2)cc1 ; Clc1ccc(C2=CC(c3cccc(Br)c3)n3ncnc3N2)cc1 ; Cc1ccc(C2C=C(c3cccc(Br)c3)Nc3ncnn32)cc1]

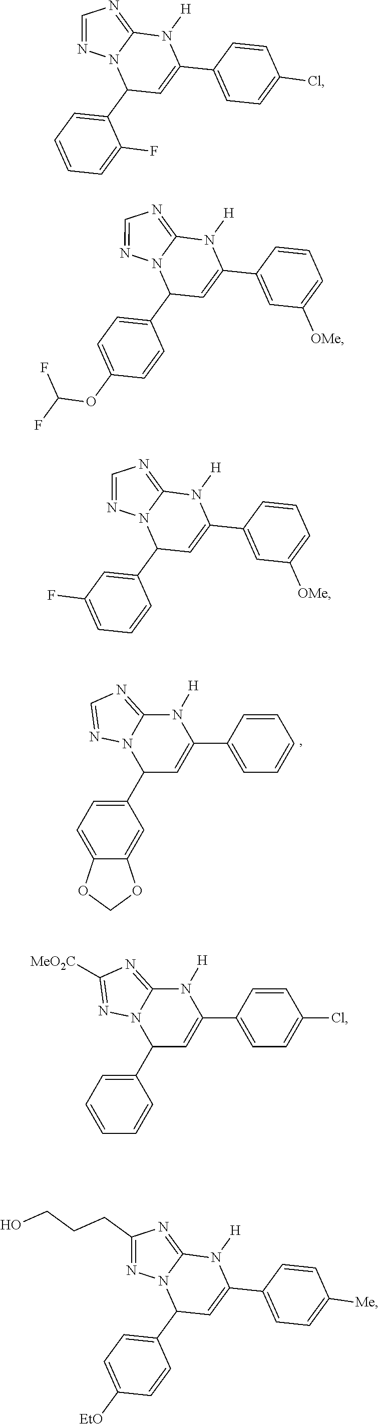 [SMILES: Fc1ccccc1C1C=C(c2ccc(Cl)cc2)Nc2ncnn21 ; COc1cccc(C2=CC(c3ccc(OC(F)F)cc3)n3ncnc3N2)c1 ; COc1cccc(C2=CC(c3cccc(F)c3)n3ncnc3N2)c1 ; C1=C(c2ccccc2)Nc2ncnn2C1c1ccc2c(c1)OCO2 ; COC(=O)c1nc2n(n1)C(c1ccccc1)C=C(c1ccc(Cl)cc1)N2 ; CCOc1ccc(C2C=C(c3ccc(C)cc3)Nc3nc(CCCO)nn32)cc1]

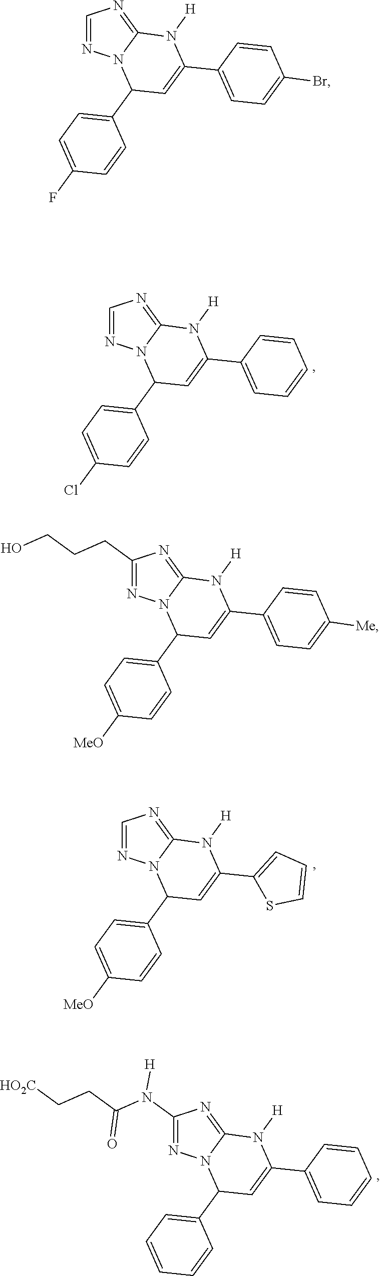 [SMILES: Fc1ccc(C2C=C(c3ccc(Br)cc3)Nc3ncnn32)cc1 ; Clc1ccc(C2C=C(c3ccccc3)Nc3ncnn32)cc1 ; COc1ccc(C2C=C(c3ccc(C)cc3)Nc3nc(CCCO)nn32)cc1 ; COc1ccc(C2C=C(c3cccs3)Nc3ncnn32)cc1 ; O=C(O)CCC(=O)Nc1nc2n(n1)C(c1ccccc1)C=C(c1ccccc1)N2]

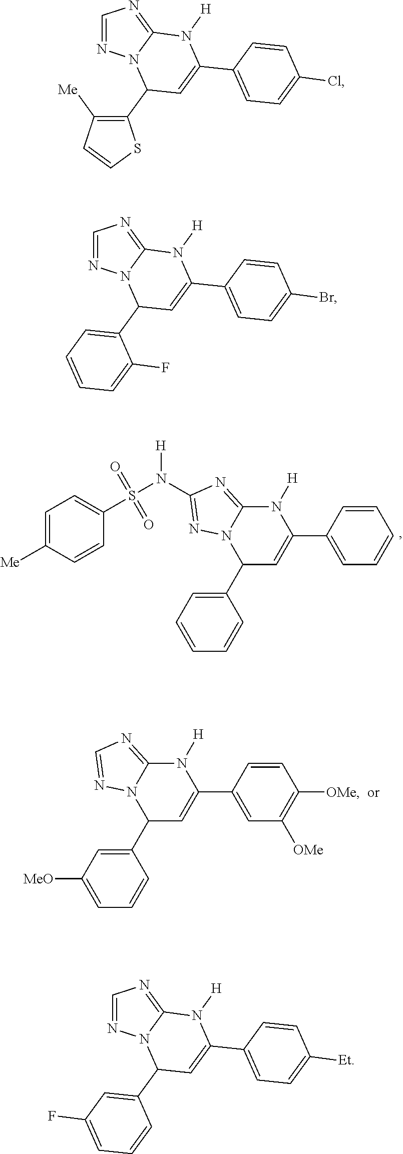

In some other embodiments, the compound of Formula (I′) or Formula (I), or a pharmaceutically acceptable salt thereof, has a structure of Formula I (c),

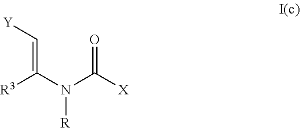

wherein
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
R is hydrogen, alkyl, aryl, cycloalkyl, or heteroaryl;
R³ is —OC(O)R¹, —C(O)OR¹, or —C(O)NR²R⁴;
R¹ is hydrogen or alkyl;
R² is hydrogen, alkyl optionally substituted with —C(O)OR¹, or aryl optionally substituted with alkyl; and
R⁴ is alkyl optionally substituted with hydroxy.

In some embodiments of compounds of Formula I (c),
X is phenyl optionally substituted with alkoxy;
Y is phenyl optionally substituted with halogen or nitro;
R is hydrogen;
R³ is —OC(O)R¹, —C(O)OR¹, or —C(O)NHR⁴;
R¹ is alkyl; and
R⁴ is alkyl substituted with hydroxy.

In some embodiments, the compound of Formula I (c) is

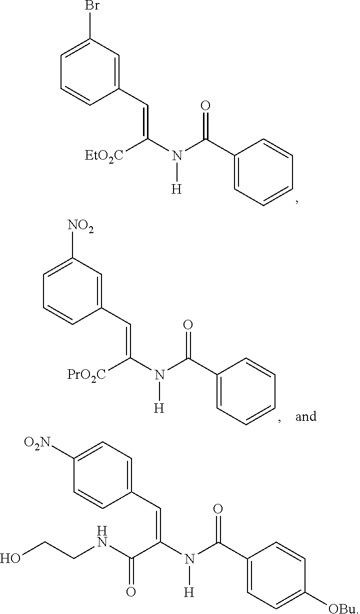

In some other embodiments, the compound of Formula (I′) or Formula (I), or a pharmaceutically acceptable salt thereof, has a structure of Formula I (d),

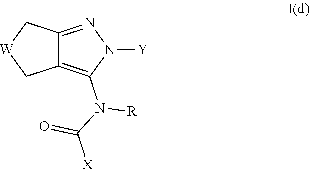

wherein
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
W is S, S(O), or S(O)₂; and
R is hydrogen, alkyl, aryl, cycloalkyl, or heteroaryl.

In some embodiments of compounds of Formula (Id),
W is S or S(O)₂;
X is alkyl;
Y is phenyl optionally substituted by halogen; and
R is hydrogen.

In some embodiments, the compound of Formula I (d) is

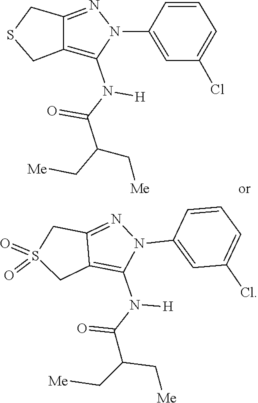

### Pharmaceutical Compositions

The compounds employed in the compositions and methods disclosed herein may be administered as pharmaceutical compositions. Such compositions may take any physical form which is pharmaceutically acceptable; illustratively, they can be orally administered pharmaceutical compositions. Such pharmaceutical compositions contain an effective amount of a disclosed compound, which effective amount is related to the dose of the compound to be administered.

In one aspect, the present disclosure provides a composition comprising the compounds as described herein, or a pharmaceutically acceptable salt thereof, and a pharmaceutically acceptable carrier.

The compositions may contain from about 0.5% to about 50% of the compound in total, depending on the desired doses and the type of composition to be used. The amount of the compound may be defined as the “effective amount”, which is the amount of the compound that provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy. In some embodiments, the pharmaceutical composition includes a compound as described herein in a range from about 0.1 to about 2000 mg, such as from about 0.5 to 500 mg or from about 1 to about 100 mg. The pharmaceutical composition may be administered to provide the compound at a daily dose of about 0.01 mg/kg to about 1000 mg/kg body weight, such as about 0.01 mg/kg to about 100 mg/kg, about 0.1 mg/kg to about 1000 mg/kg, about 0.1 to about 500 mg/kg, about 0.1 to about 100 mg/kg, or about 50 to about 100 mg/kg body weight.

In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 UM, 0.01 μM, 0.5 μM, 0.1 μM, 1.0 μM, 10 μM, and 100 μM (e.g., 0.1 μM-1.0 μM).

The term “pharmaceutically acceptable” means approved by a regulatory agency of the Federal or a state government or listed in the U.S. Examples of suitable pharmaceutical carriers are described in “Remington's Pharmaceutical Sciences” by E. W. Martin. Suitable pharmaceutically acceptable carriers include, but are not limited to, for example, suitable diluents, vehicles, excipients, preservatives, solubilizers, emulsifiers, liposomes, or nanoparticles, among others. Additionally, such pharmaceutically acceptable carriers may be aqueous or non-aqueous solutions, suspensions, and emulsions. Examples of nonaqueous solutions include propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The formulation should be selected according to the mode of administration. The compositions may include a pharmaceutical carrier, excipient, or diluent, which are nontoxic to the subject being exposed thereto at the dosages and concentrations employed. Examples of pharmaceutical carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN brand surfactant, polyethylene glycol (PEG), and PLURONICS™ surfactant.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

Suitable formulations include those that are suitable for more than one route of administration. For example, the formulation can be one that is suitable for both oral and intravenous administration. Alternatively, suitable formulations include those that are suitable for only one route of administration as well as those that are suitable for one or more routes of administration, but not suitable for one or more other routes of administration. For example, the formulation can be one that is suitable for oral, topical, transdermal, percutaneous, intravenous, intramuscular, intranasal, buccal, and/or intrathecal administration but not suitable for intracerebral administration.

The inert ingredients and manner of formulation of the pharmaceutical compositions may be selected from conventional technologies. The usual methods of formulation used in pharmaceutical science may be used here. Suitable types of compositions include, but are not limited to, tablets, chewable tablets, capsules, solutions, parenteral solutions, intranasal sprays or powders, troches, suppositories, transdermal patches, and suspensions.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders, but any suitable capsule formulation can be used.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

The composition can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the compound is pumped by osmotic action.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

The composition can be prepared with materials (e.g., actives excipients, carriers (such as cyclodextrins), diluents, etc.) having properties (e.g., purity) that render the formulation suitable for administration to humans or non-human subjects. In some embodiments, the composition is suitable for use in humans. In some embodiments, the composition is prepared with materials having purity and/or other properties that render it suitable for administration to non-human subjects, but not suitable for administration to humans.

Each dosage unit may contain the dose of a given compound, for example, a daily dose, or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The composition may include a single compound or a combination of compounds as described herein for administration. For example, a two or more of the compounds described herein may be administered for an anti-parasitic effect. In addition, the composition may include solvate forms of the compounds or salts, esters, and/or amides, thereof. Solvate forms may include ethanol solvates, hydrates, and the like.

The disclosed compounds or pharmaceutical compositions comprising the disclosed compounds may be administered with additional therapeutic agents. The additional therapeutic agent may include, for example, one or more known agent for treating parasitic infection. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical compositions are formulated to comprise the disclosed compounds and further to comprise the one or more additional therapeutic agents.

### Methods of Use

The compounds as described herein may effectively inhibit the growth and propagation of parasites in vivo, in particular the growth and propagation of flatworms within a subject. In one aspect, the present disclosure provides a method of treating a parasitic infection in a subject in need thereof, the method comprises administering to the subject an effective amount of the compounds as described herein, or a pharmaceutically acceptable salt thereof, or the composition as described herein. In use, the compounds as described herein may be cytotoxic to the parasite but not to the subject being treated.

Suitable subjects include mammals and non-mammals. Mammals include any member of the class Mammalia. Examples of mammals include, but are not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, guinea pigs, and the like. Examples of non-mammals include, but are not limited to, birds, fish, crustaceans, among others. A subject can be any suitable age or sex. In some embodiments, the subject is a human. In some embodiments, the subject is a non-human subject, such as a fish, a bird, a dog, a cat, or a livestock.

As used herein, “treating” or “treatment” describes the management and care of a subject for combating the disease, condition, or disorder. Treating includes the administration of the compound or composition described herein to reduce, prevent, ameliorate and/or improve the onset of the symptoms or complications, alleviating the symptoms or complications, or reducing or eliminating the disease, condition, or disorder associated with a parasitic infection, such as a flatworm parasitic infection. The term treating as described herein includes the inhibiting or reducing growth and/or propagation/reproduction of parasites within a subject (i.e., in vivo).

Symptoms of parasitic infections include, but are not limited to, for example, stomach cramps and pain, nausea or vomiting, dehydration, weight loss, swollen lymph nodes, digestive problems including unexplained constipation, diarrhea or persistent gas, skin issues such as rashes, eczema, hives, and itching, continuous muscle and joint pain, fatigue, depression or feeling of apathetic, constant hunger, iron deficiency/anaemia, grinding teeth during sleep, unexplained feelings of anxiety, recurrent yeast infections, itching of the anus or vagina, itching, redness, irritation, and an unusual discharge from the genital area, trouble falling asleep or waking up multiple times during the night.

As used herein the term “effective amount” or “therapeutically effective amount” refers to the amount or dose of the present compound or composition, which, upon single or multiple dose administration to the subject, provides the desired effect in the subject under diagnosis or treatment. The disclosed methods may include administering an effective amount of the disclosed compounds (e.g., as present in a pharmaceutical composition) for treating a disease or disorder, e.g., a parasitic infection.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

The term “administering” or “administration” refers to introducing the present compounds or compositions into the body of the subject, such as by oral delivery. Suitable routes of administration include, but are not limited to, oral, topical, transdermal, buccal, sublingual, pulmonary, transdermal, transmucosal, rectal, as well as subcutaneous, intraperitoneal, intravenous, and intramuscular injection.

The methods of treating a parasitic infection include treating a parasitic flatworm infection. In some embodiments, the parasitic infection is a parasitic flatworm infection. Suitable flatworm infections contemplated to be treated using the compound and compositions described herein include, but are not limited to, trematode (i.e. flukes, such as schistosoma species (blood fluke), Fasciola species (liver fluke), Paragonimus westermani (lung fluke), clonorchis sinensis (liver fluke), Opisthorchis viverrini (liver fluke)), cestode (i.e. tapeworms, such as pork tapeworm (taenia solium), beef tapeworm (taenia saginata), Echinococcus species, Dipylidium caninum (heartworm), Diphyllobothrium species, Gyrodactcylus species, and dwarf tapeworm (hymenolepis nana)), turbellaria (planarians), monogenean (i.e. monopisthocotyleans and the polyopisthocotyleans), and turbellaria (such as temnocephala lamothei, dugesia aenigma, dugesia bifida, planaria simplex, girardia dorotocephala, girardia tigrina). In particular embodiments, the present method can be used to treat a parasitic flatworm infection, in which the parasitic flatworm is trematode, cestode, monogenean, schistosome, tapeworm, or a combination thereof.

The benzamidoquinazolinone compounds as disclosed herein may provide treatment for a disease caused by parasitic worms in multiple species (such as humans), including fasciolosis, schistosomiasis, and other diseases. Schistosomiasis is a disease caused by parasitic flatworms, namely, schistosomes. In another aspect, the present disclosure provides a method of treating schistosomiasis in a subject in need thereof, the method comprises administering to the subject an effective amount of the compounds as described herein, or a pharmaceutically acceptable salt thereof, or the composition as described herein.

The present compounds may activate a flatworm transient receptor potential (TRP) channel, which may in turn mediate sustained Ca²⁺ influx and worm paralysis. Thus, the disclosed compounds may act as ion channel activators to treat parasitic flatform infections. In another aspect, the present disclosure provides a method of activating a transient receptor potential channel of a parasitic flatworm, the method comprising introducing to the parasitic flatworm an effective amount of the compounds as described herein, or a salt thereof, or the composition as described herein. In some embodiments, the transient receptor potential channel is a member of the melastatin family (TRPM), such as TRPM1, TRPM2, TRPM3, TRPM4, TRPM5, TRPM6, TRPM7, and TRPM8.

The present compound may be used to effectively control infection or growth of a parasitic flatworm. In another aspect, the present disclosure provides a method of causing paralysis or inhibiting growth of a parasitic flatworm, the method comprising introducing to the parasitic flatworm an effective amount of the compounds as described herein, or a salt thereof, or the composition as described herein.

For the present methods, the compounds as described herein, or a salt thereof, or the composition as described herein may be introduced to a parasitic flatworm that is in an infected subject (e.g., to inhibit the growth of, or treat an infection caused by, the parasitic flatworm in the subject). In some embodiments, the subject is a human. Alternatively, the compound or composition may be introduced to a parasitic flatworm that is in an environment (outside the body of a subject), such as a river, a lake, a water reservoir, a grassland, or a forest. In these cases, the effective amount of the compound or composition refers to an amount that provides desired effect of activating a TRP channel and/or causing paralysis or inhibiting growth of the parasitic flatworm in such environment.

The compounds utilized in the methods disclosed herein may be administered in conventional dosage forms prepared by combining the active ingredient with standard pharmaceutical carriers or diluents according to known procedures, including for example mixing, granulating, compressing, and/or dissolving various ingredients as appropriate to the desired preparation.

In some embodiments, the subject may be administered a dose of the disclosed compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of the disclosed compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as endpoints any of these disclosed doses (e.g., 2.5 mg-200 mg).

### Kits

In another aspect, the present disclosure provides a kit comprising a pharmaceutical composition comprising the compounds as disclosed herein and instructional material.

The term “instructional material” refers to a publication, a recording, a diagram, or any other medium of expression which is used to communicate the usefulness of the present pharmaceutical composition for one of the purposes set forth herein in a human. The instructional material can also, for example, describe an appropriate dose of the present pharmaceutical composition. The instructional material of the present kit can, for example, be affixed to a container which contains a pharmaceutical composition as disclosed herein or be shipped together with a container which contains the pharmaceutical composition. Alternatively, the instructional material can be shipped separately from the container with the intention that the instructional material and the pharmaceutical composition be used cooperatively by the recipient.

It should be apparent to those skilled in the art that many additional modifications beside those already described are possible without departing from the inventive concepts. Other features and advantages of the invention will be apparent from the present description and claims. Although methods and materials similar or equivalent to those described herein can be used in the practice or testing of the present disclosure, suitable methods and materials are described below. In addition, the materials, methods, and examples are illustrative only and not intended to be limiting.

### EXAMPLES

The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### Example 1: Novel Chemotypes Identified

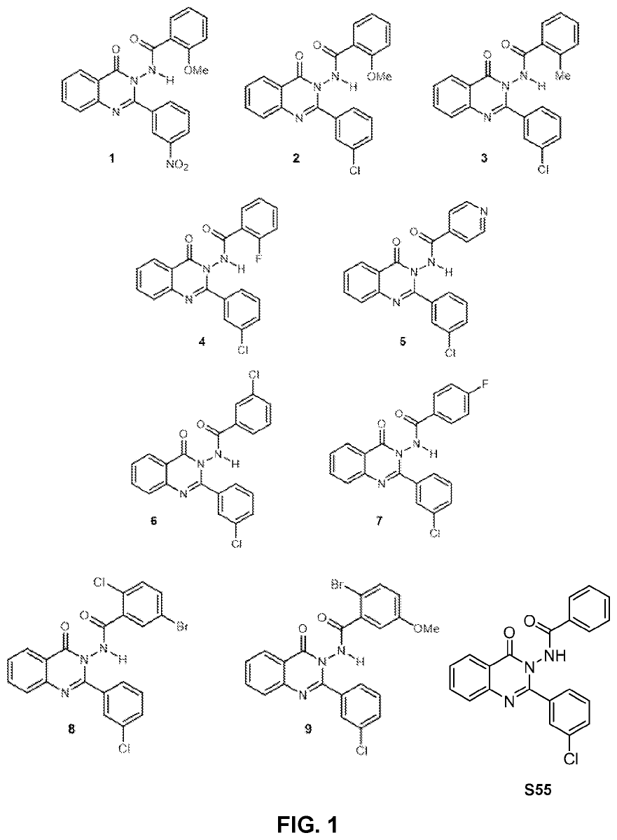

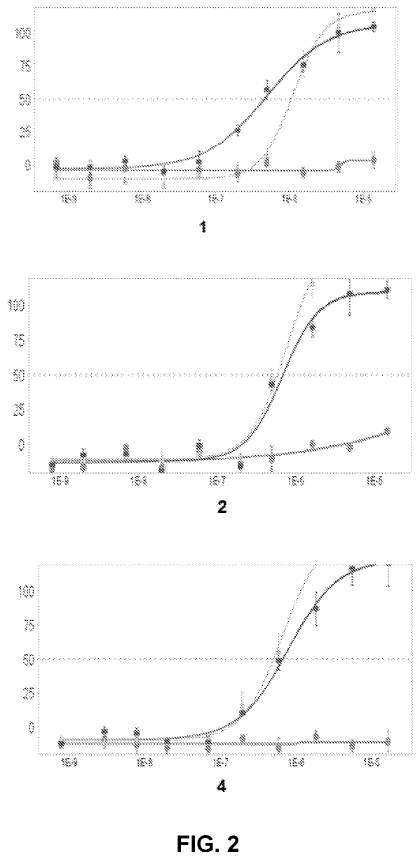

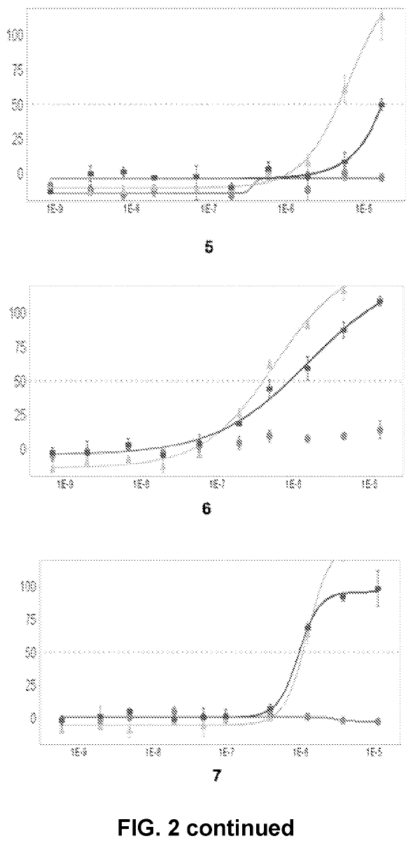

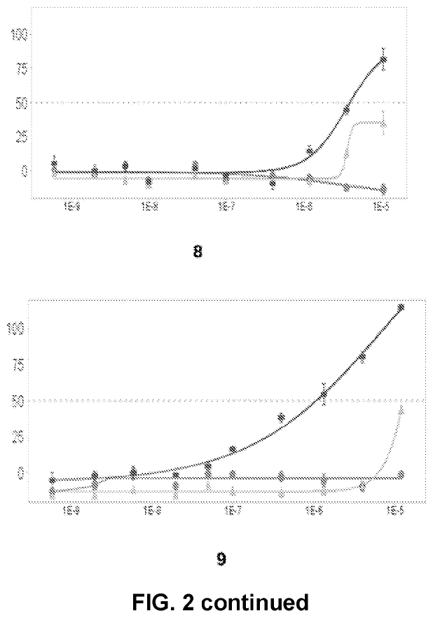

Exemplary, non-exhaustive, list of the compounds of the present disclosure are shown in FIG. 1. The dose-response curves for the some of these compounds are shown in FIG. 2. HEK cells were transfected with cDNA encoding Sm.TRPM_{PZQ} or the TRPM_{PZQ} from F. hepatica (Fh.TRPM_{PZQ}) and incubated overnight at 37° C. Briefly: the HEK cells were resuspended in electroporation buffer. Plasmid DNA was added to the cells in buffer, and this mixture was loaded into the appropriate cassette, inserted into the machine, and the cells were electroporated and then frozen. On the day of the assays, cells were thawed in a 37° C. water bath and resuspended in DMEM+10% FBS followed by pelleting in the usual way. The media was aspirated, and the remaining pellet was resuspended in Ix HBSS with 20 mM HEPES and 1% DMSO. This cell suspension was then dispensed into 1536 well plates at a density of 4,000 cells/well. Measurements of fluorescence intensity were made using a FLIPR. A read of basal fluorescence values was made prior to addition of compounds or controls. Responses compared on a per plate basis by evaluating the percentage response of each compound versus the high control (response to EC₁₀₀ PZQ (10 μM) corrected for low control (response to DMSO). Titration assays were executed as 10-point concentration-response curves, performed in triplicate. Counter-screening was performed as a negative control using untransfected HEK293 cells for comparison with results testing the same compounds effect on cells expressing Sm.TRPMPZQ or Fh.TRPMPZQ. For each compound, agonist activity was plotted against compound concentration and data were fitted with a sigmoidal function. The dark blue curve represents response of Sm.TRPMPZQ to agonist. The light blue curve represents response of Fh.TRPM_{PZQ} to agonist. The red curve represents response of untransfected HEK293 cells to agonists.

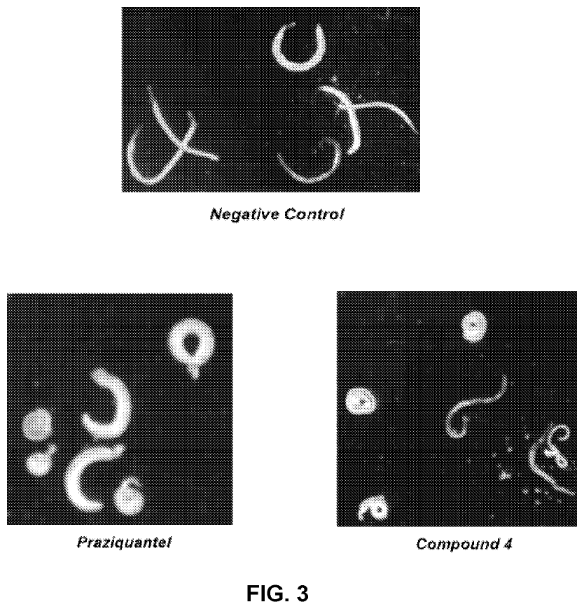

It was observed that N-amidoquinazolinones contract S. mansoni in a manner similar to the channel activator (and standard of treatment) praziquantel. The results are shown in FIG. 3. Adult schistosomes were harvested from the mesenteric vasculature of female Swiss Webster mice previously infected (˜49 days earlier). Harvested schistosomes were washed in RPMI 1640 supplemented with 5% heat inactivated FBS, HEPES, and penicillin-streptomycin. After isolation, worms were incubated overnight in vented petri dishes. Movement assays were performed using male and female worms in six well dishes (˜5 individual worms/3 ml media per well). Schistosomes were treated with a molecule such as compound 4, and video recordings of worm motility were captured using a stereomicroscope coupled to a CCD camera. This figure shows that Compound 4 causes contraction and paralysis in the same way that known molecule PZQ does.

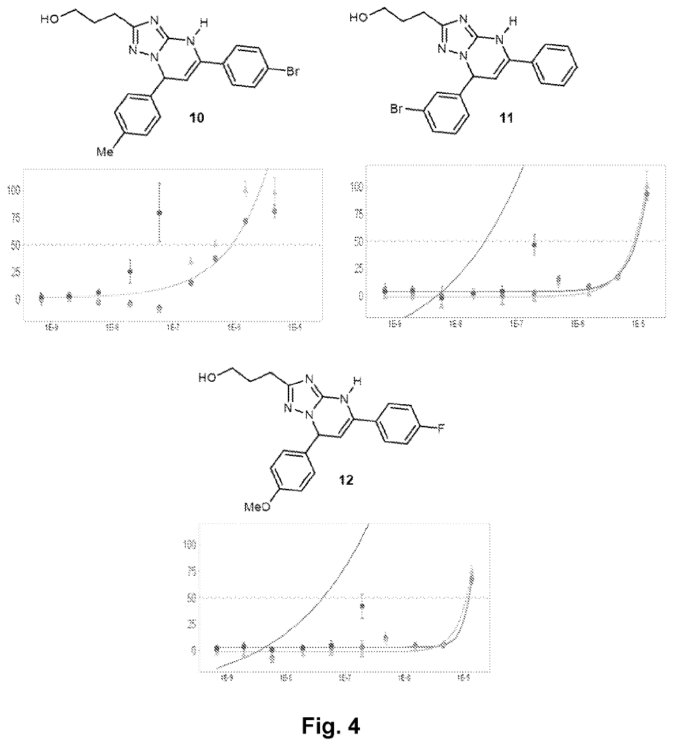

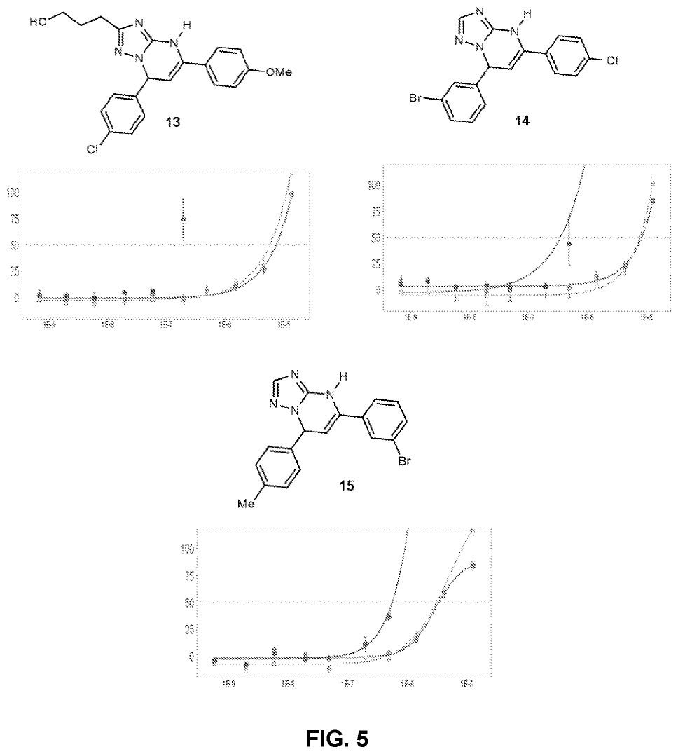

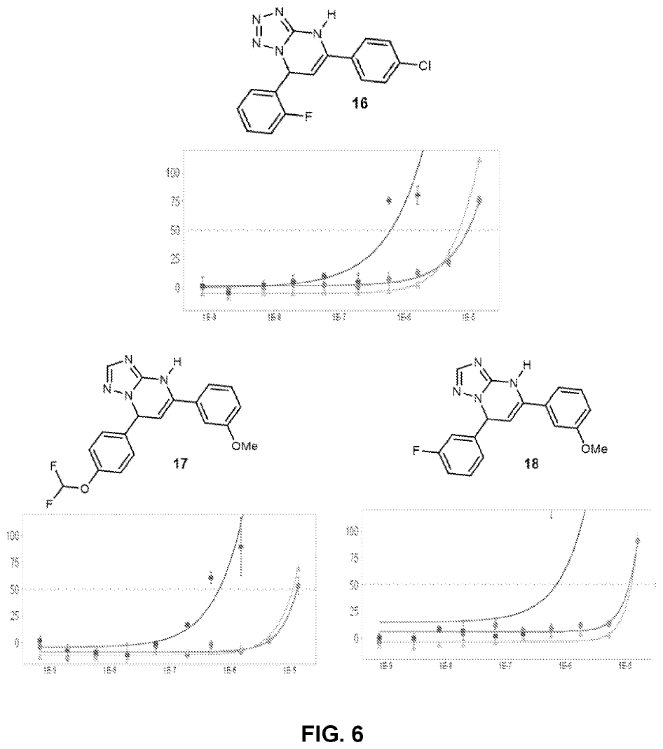

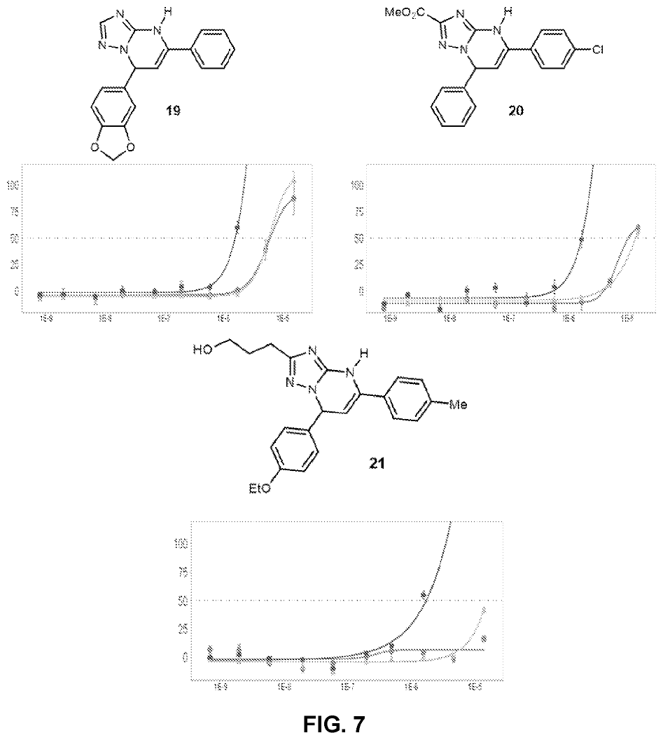

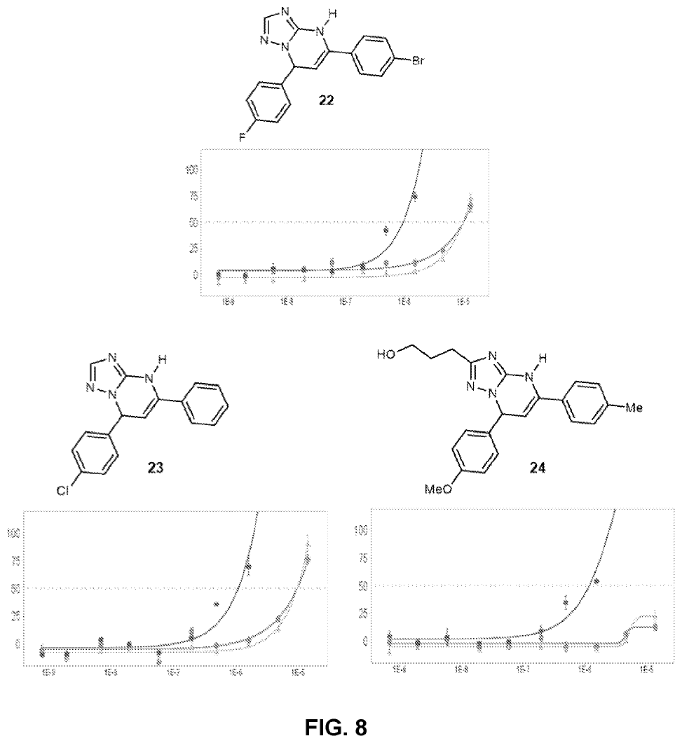

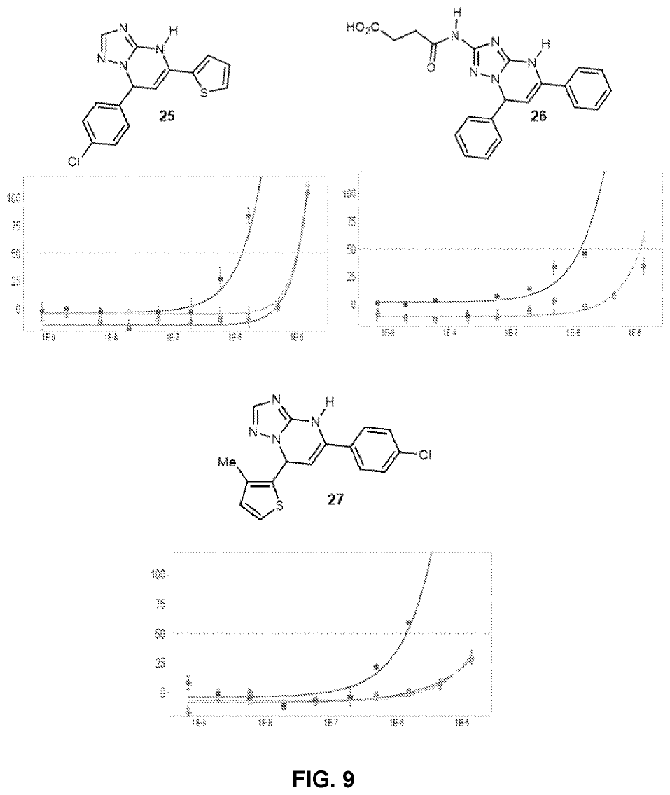

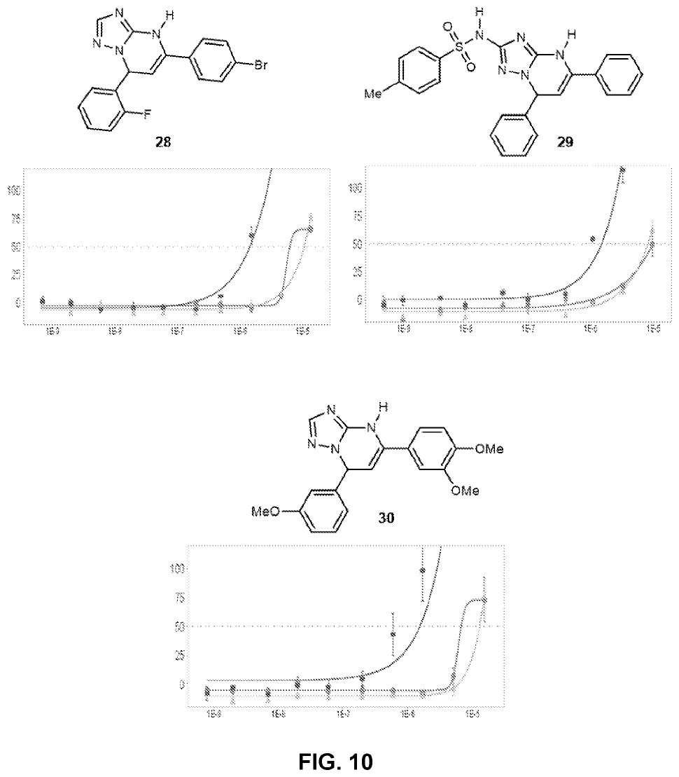

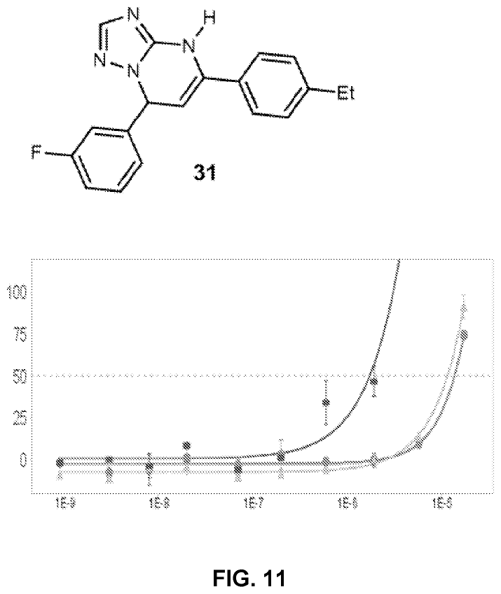

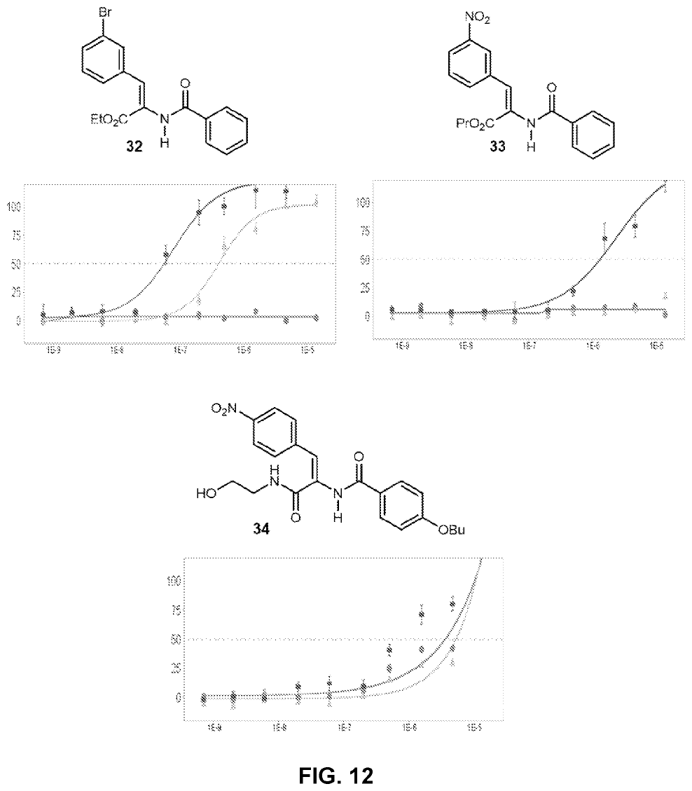

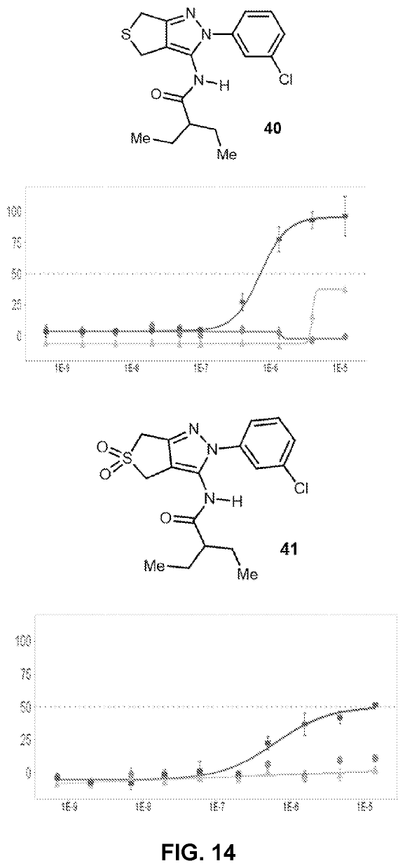

Additionally, the chemical structures listed in FIGS. 4-12 and FIG. 14 depict brief, but non-exhaustive examples of the present disclosure. The dose response curves were obtained as in FIG. 2. Briefly, HEK293 cells were transfected with cDNA encoding Sm.TRPM_{PZQ} or Fh.TRPM_{PZQ} and incubated overnight at 37° C. The HEK293 cells were resuspended in electroporation buffer. Plasmid DNA was added to the cells in buffer, and this mixture was loaded into the appropriate cassette, inserted into the machine, and the cells were electroporated and then frozen. On the day of the assays, cells were thawed in a 37° C. water bath and resuspended in DMEM+10% FBS followed by pelleting in the usual way. The media was aspirated, and the remaining pellet was resuspended in Ix HBSS with 20 mM HEPES and 1% DMSO. This cell suspension was then dispensed into 1536 well plates at a density of 4,000 cells/well. Measurements of fluorescence intensity were made using a FLIPR. A read of basal fluorescence values was made prior to addition of compounds or controls. Responses compared on a per plate basis by evaluating the percentage response of each compound versus the high control (response to EC100 PZQ (10 μM) corrected for low control (response to DMSO). Titration assays were executed as 10-point concentration-response curves, performed in triplicate. Counter-screening was performed as a negative control using untransfected HEK293 cells for comparison with results testing the same compounds effect on cells expressing Sm.TRPMPZQ or Fh.TRPMPZQ. For each compound, agonist activity was plotted against compound concentration and data were fitted with a sigmoidal function. The dark blue curve represents response of Sm.TRPMPZQ to agonist. The light blue curve represents response of Fh.TRPMPZQ to agonist. The red curve represents response of untransfected HEK293 cells to agonists.

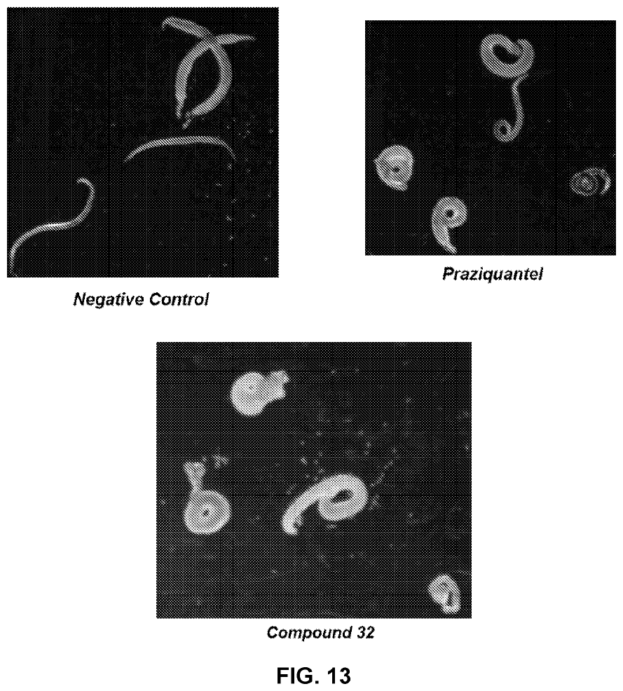

It was also observed that 2-amidoacrylates or 2-amidoacrylamides contract S. mansoni in a manner similar to the channel activator (and standard of treatment) praziquantel. The results are shown in FIG. 13. The worm mobility assay performed in FIG. 13 is same as that performed in FIG. 3. Briefly, adult schistosomes were harvested from the mesenteric vasculature of female Swiss Webster mice previously infected (˜49 days earlier). Harvested schistosomes were washed in RPMI 1640 supplemented with 5% heat inactivated FBS, HEPES, and penicillin-streptomycin. After isolation, worms were incubated overnight in vented petri dishes. Movement assays were performed using male and female worms in six well dishes (˜5 individual worms/3 ml media per well). Schistosomes were treated with a molecule such as compound 32, and video recordings of worm motility were captured using a stereomicroscope coupled to a CCD camera. This figure shows that Compound 32 causes contraction and paralysis in the same way that known molecule PZQ does.

### Example 2: Activity of Compound BZQ/S55

Infection with F. hepatica causes massive disease in humans, and is also responsible, worldwide, for >$3 billion USD in agricultural losses annually. The standard of treatment for this infection is triclabendazole. However, there is increasing resistance in the field to triclabendazole, and this resistance is estimated to be as high as 60% in certain regions. Given this resistance, and the importance of the infection to human health and agriculture, there is a need for new flukicides. The development of new molecules targeting novel druggable targets is essential to reach this goal. To this end, a first-in-class molecule, N-(2-(3-chlorophenyl)-4-oxoquinazolin-3(4H)-yl)benzamide (also referred to as “S55” herein, structure shown below) was identified along with other benzamidoquinazolinones (BZQ) compounds that are active against F. hepatica. These molecules function via activation of Fh.TRPM_{PZQ}, a new target that now proves druggable. These molecules function in a dose-dependent manner and effect rapid contraction of both immature and adult liver flukes. Significantly, these molecules retain complete efficacy against triclabendazole-resistant liver flukes. The benzamidoquinazolinones are also effective against schistosomiasis both ex vivo and in vivo, providing the opportunity for further development of broad- or pan-spectrum treatment of parasitic diseases. Therefore, the benzamidoquinazolinone class of molecules, and other analogs may provide treatment for fasciolosis, schistosomiasis, and other diseases caused by parasitic worms in multiple species including humans.

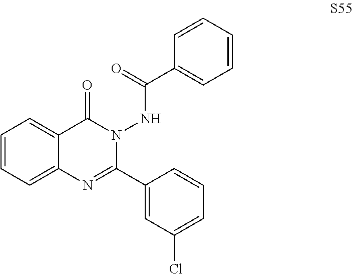 [SMILES: O=C(Nn1c(-c2cccc(Cl)c2)nc2ccccc2c1=O)c1ccccc1]

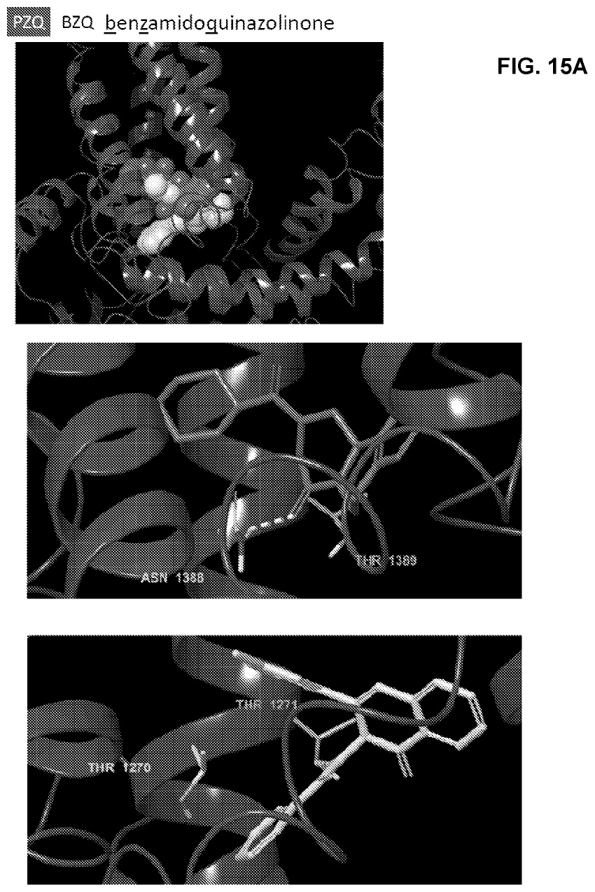

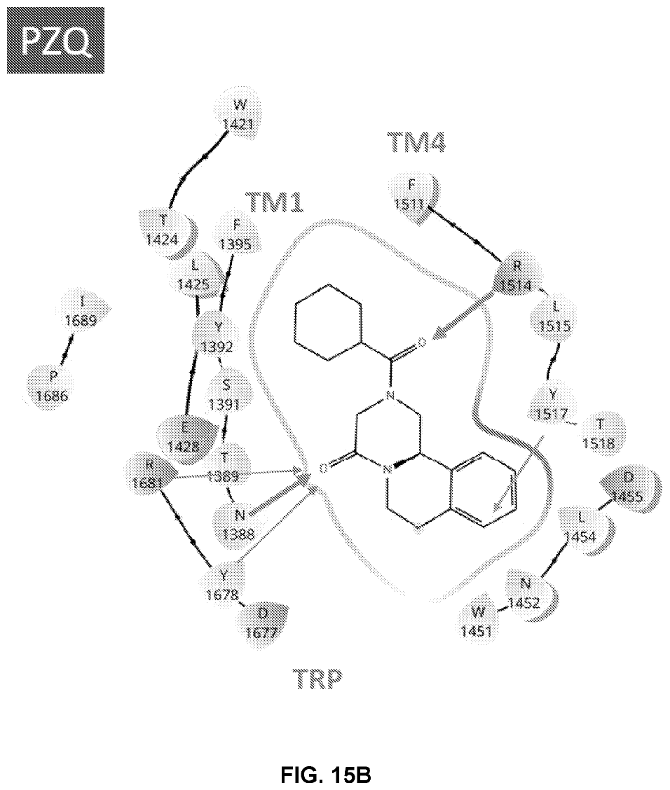

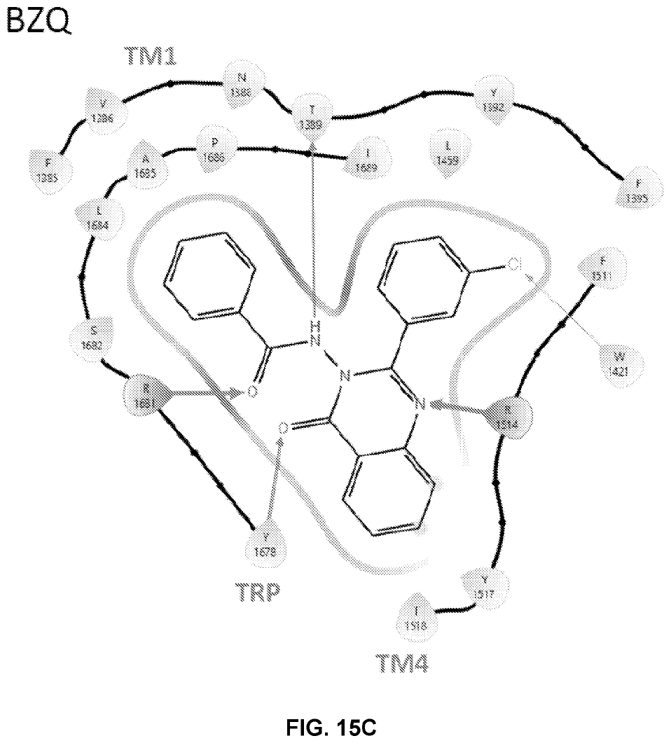

A putative binding pose of praziquantel and BZQ/S55 in Sm.TRPM_{PZQ} and Fh.TRPM_{PZQ}: The homology model of Sm.TRPM_{PZQ} was prepared using the Schrodinger Computational Suite as previously reported (Park et. al., Sci. Tran. Med., 2021). For the Fasciola hepatica model, the previously described Schistosoma mansoni model was used as a template, and the model was generated with the sequence for Fh.TRPM_{PZQ} previously reported (Rohr et. al., PNAS, 2023). Results generated from Induced-Fit docking reveal that BZQ binds in a different pose than praziquantel (FIG. 15A). This allows for activation of the TRPM_{PZQ} channel in multiple species. Specifically, the N—H of BZQ binds to the conserved T1389 (Sm.TRPM_{PZQ})/T1271 (Fh.TRPM_{PZQ}) (FIG. 15C). This is in stark contrast to the pose of praziquantel which requires residue N1388 (Sm.TRPM_{PZQ})/T1270 (Fh.TRPM_{PZQ}) to be a hydrogen bond donating residue other than threonine (FIG. 15B) (N, H, S are all tolerated in both species, allowing activation by both PZQ and BZQ). Otherwise, interactions remain conserved; R1514 (Sm.TRPM_{PZQ}) and its corresponding residue in other species is necessary for activation. Likewise, Y1678 (Sm.TRPM_{PZQ}) and its corresponding residue in other species is required, and R1681 (Sm.TRPM_{PZQ}) and its corresponding residue in other species both form interactions with the TRPM_{PZQ} binding pockets (FIG. 15C).

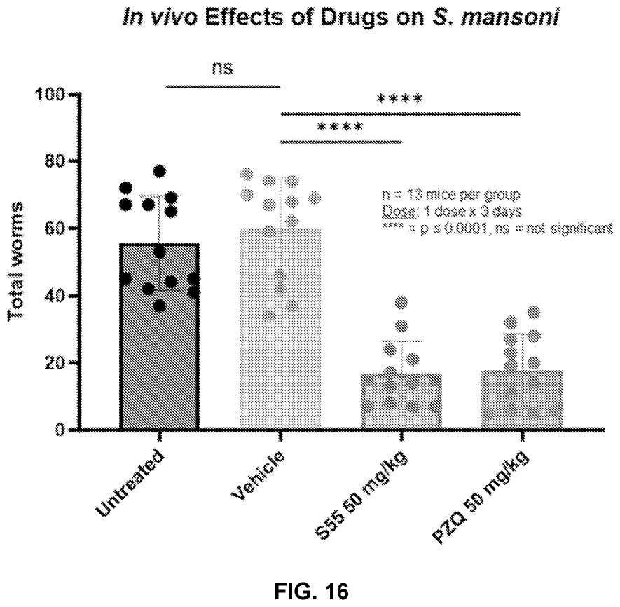

BZQ/S55 is as effective as Praziquantel in vivo in a murine model of schistosomiasis: Female Swiss Webster mice were infected with S. mansoni cercariae (NMRI strain) by the Schistosomiasis Resource Center at the Biomedical Research Institute (Rockville, MD). All animal experiments followed ethical regulations approved by the MCW IACUC committee. At 7 weeks post-infection, mice were randomly sorted into 4 groups of 13 mice. One group was left untreated as a control. A second group was treated with vehicle (a mixture of DMSO/PEG₄₀₀/H₂O). A third group was treated with praziquantel (50 mg/kg, intraperitoneal, 1× daily) for 3 days in a row. In addition, a fourth group was treated with BZQ/S55 in the same fashion as praziquantel. Four days after the first treatment, the mice were euthanized, and the mesenteric vasculature was dissected in the usual fashion to recover schistosomes. The worm burden from each mouse was individually plotted (FIG. 16). There was no significant difference between untreated mice and mice treated with vehicle, confirming that the vehicle the drugs were dosed in had no effect on worm burden. In contrast, there was a significant reduction in worm burden in the mice treated with both praziquantel and BZQ/S55, and there was no difference found between these two groups. Further, no toxicity was noticed. These findings indicate that S55 is an effective molecule for treating schistosomiasis, and is as effective as praziquantel, the standard of treatment (FIG. 16).

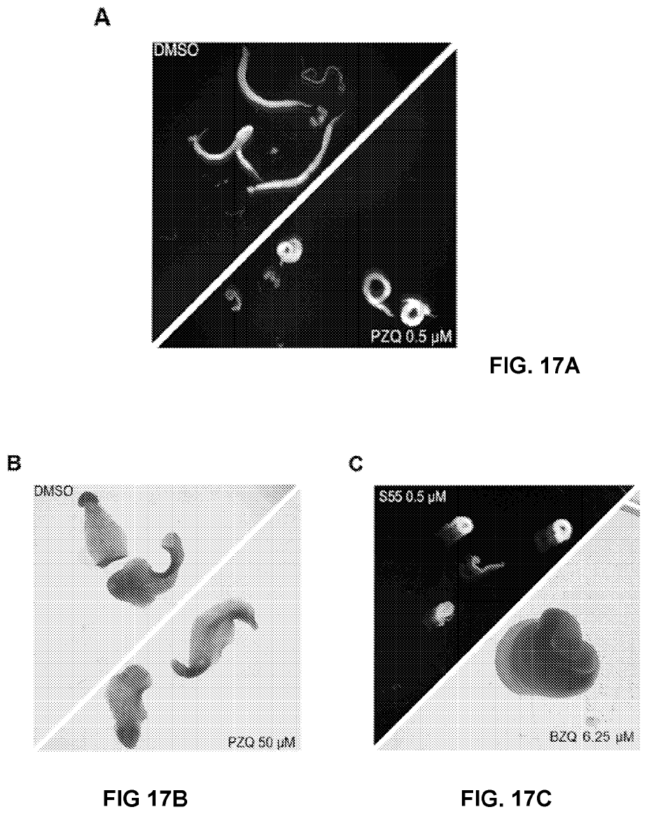

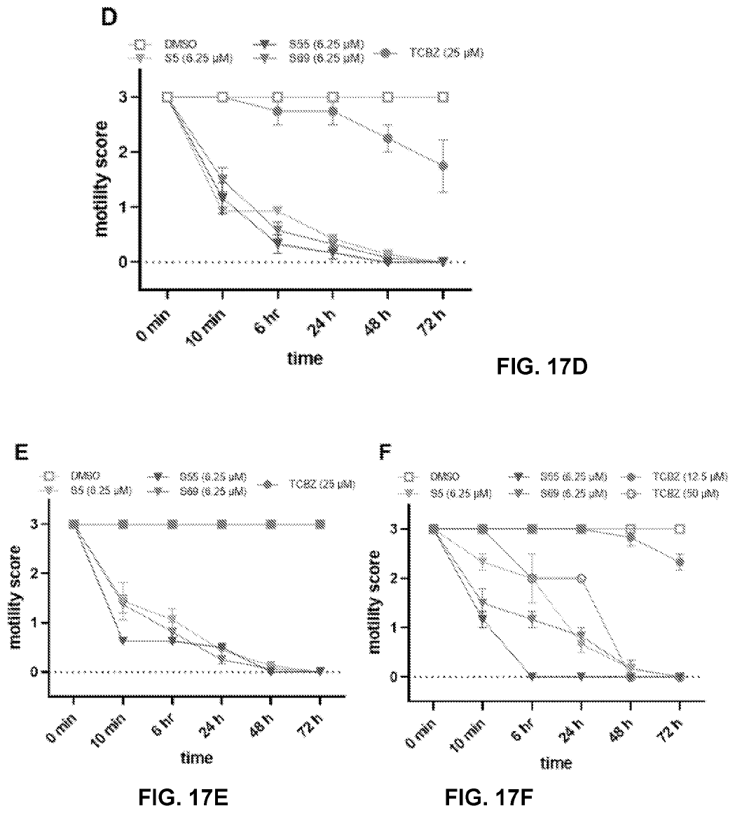

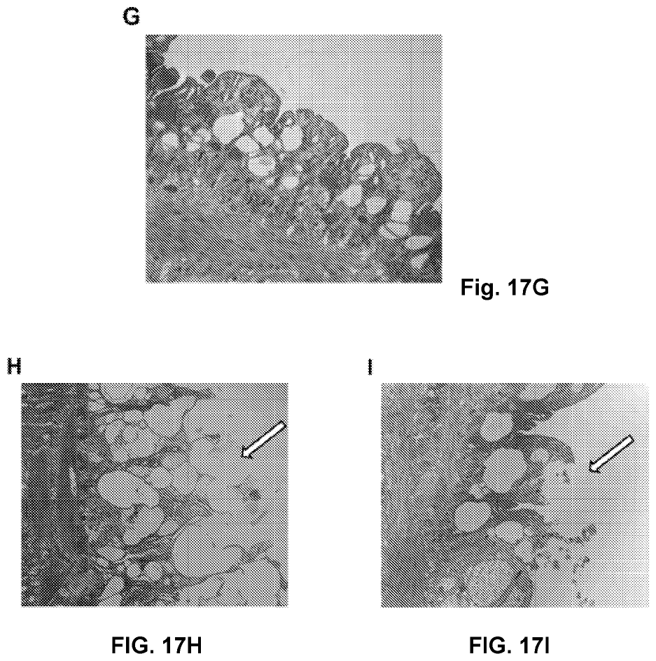

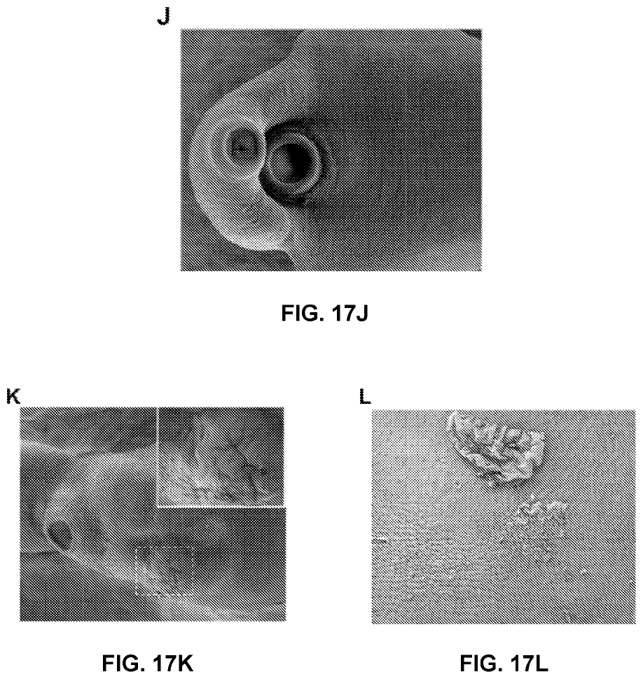

BZQ/S55 is active ex vivo on Fasciola hepatica adults and immature worms and mimics praziquantel on schistosomes: Immature and adult F. hepatica were obtained as previously reported (Morawietz et. al., Frontiers in Veterinary Science, 2020). Briefly, Male Wistar rats served as the final host to obtain immature and adult stages of F. hepatica after infection with metacercariae from an Italian strain of F. hepatica. Immature flukes were collected from livers 4 weeks post-infection. Adult flukes were collected from bile ducts at 12 weeks post-infection. Worms were kept for 1 h in normal saline to allow clearance of gut contents and then used for experiments. In vitro experiments were performed utilizing a similar procedure as previously reported. Excised worms were treated with different concentrations of praziquantel, triclabendazole, BZQ/S55, S5, and S69, or DMSO as a negative control. The flukes were incubated at 37° C. in a 5% CO₂ atmosphere for 72 h, and inhibitor-induced effects on worm viability were assessed using a stereomicroscope at 10× magnification. Worm motility was assessed using the following scores: 3 (normal motility), 2 (reduced motility), 1 (minimal and sporadic movements), and 0 (no movement even upon mechanical stimulation with forceps; considered dead). Representative results from these studies are shown FIGS. 17A-L.

### Example 3: Chemical Synthesis

### General Scheme for the Synthesis of Antiparasitic Benzamidoquinazolinones

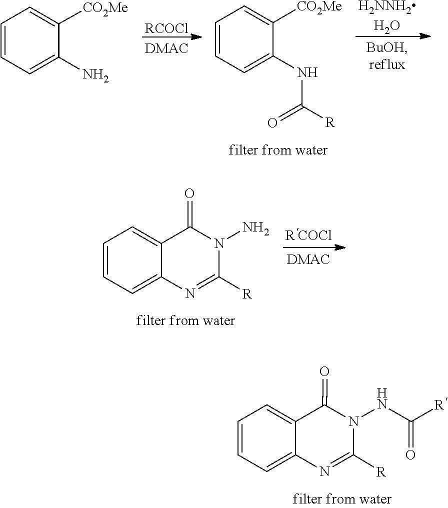

### Synthetic Chemistry Procedures

All reagents and solvents were commercial grade and purified prior to use if necessary. Thin layer chromatography (TLC) was performed using glass-backed silica gel (250 μm) plates or glass-backed Biotage® KP-NH plates. UV light, and/or the use of potassium iodoplatinate, potassium permanganate and ninhydrin stains were used to visualize products. MPLC was performed on a Biotage Isolera in conjunction with a Biotage Dalton 2000 using the conditions indicated. Nuclear magnetic resonance spectra (NMR) were acquired on a Bruker AV-III-500 (500 MHz) spectrometer equipped with a TCI cryoprobe. Chemical shifts were measured relative to residual solvent peaks as an internal standard set to δ 7.26 and δ 77.0 (CDCl₃) or 2.50 and 39.5 (DMSO-d₆). All reported compounds were >95% pure by 1H NMR analysis. High-resolution mass spectra (HRMS) were recorded on a Thermo Scientific Orbitrap XL spectrometer by use of the indicated ionization method. A post-acquisition gain correction was applied using reserpine as a lock mass.

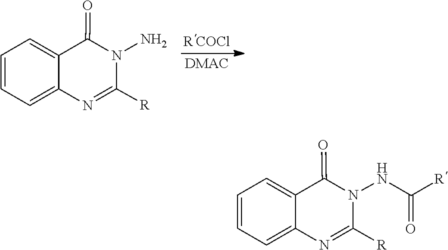

General Procedure A. To a solution of the amine in N,N-dimethylacetamide at ambient temperature was added the acid chloride dropwise, and the reaction was stirred at ambient temperature for 16 h. The reaction was poured into H₂O, and the precipitated solid was collected by filtration, washed with water, and dried in vacuo to afford the product.

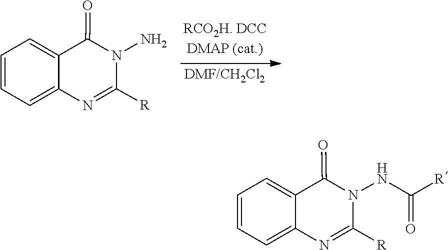

General Procedure B. To a solution of the amine, acid, and DMAP in DMF was added DCC and the reaction was stirred in a heating mantle at 60° C. for 18 h. The reaction was concentrated in vacuo, and the residue was suspended in CH₂Cl₂ and filtered through Celite®). The filtrate was washed with sat aq NaHCO₃, sat aq NH₄Cl, and brine, and dried and concentrated. MPLC afforded the desired product.

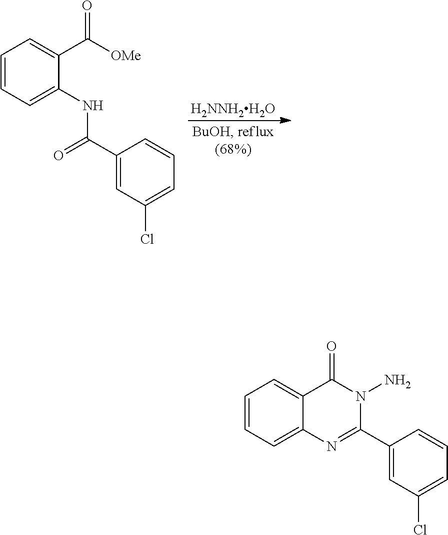

3-Amino-2-(3-chlorophenyl)quinazolin-4 (3H)-one (S11). A suspension of the ester (15.5 g, 53.5 mmol) in H₂NNH₂·H₂O (110 mL) and 1-butanol (110 mL) was refluxed in a heating mantle for 24 h. After cooling to ambient temperature, the reaction was poured into H₂O (4 L) and the precipitate was filtered and dried in vacuo to afford the product as a free-flowing, colorless solid (9.85 g, 68% yield). ¹H NMR (500 MHZ, DMSO-d₆) δ 8.19 (d, J=7.4 Hz, 1H), 7.87 (br s, 1H), 7.84 (br dd, J=7.6, 1.2 Hz, 1H), 7.76 (d, J=7.6 Hz, 1H), 7.73 (d, J=8.1 Hz, 1H), 7.61-7.55 (br m, 2H), 7.51 (dd, J=7.8 Hz, 1H), 5.65 (s, 2H); ¹³C NMR (125 MHz, DMSO-d₆) ppm 162.5, 155.9, 147.9, 138.1, 135.7, 133.4, 130.8, 130.74, 130.70, 129.6, 128.8, 128.4, 127.4, 121.6.

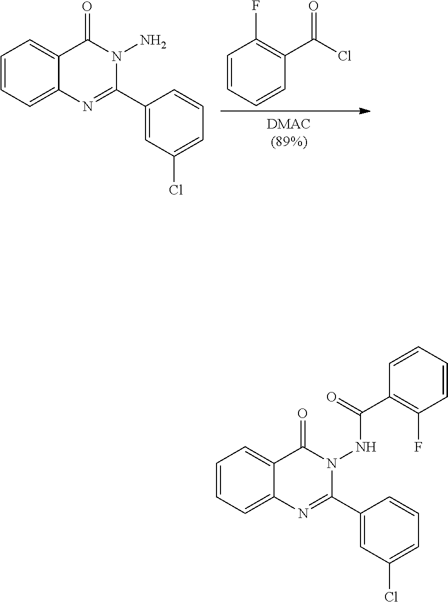

N-(2-(3-Chlorophenyl)-4-oxoquinazolin-3(4H)-yl)-2-fluorobenzamide. Following General Procedure A, the amine (1.00 g, 3.68 mmol) and acid chloride (528 μL, 4.42 mmol) in N,N-dimethylacetamide (40 mL), after filtration, afforded the amide as a colorless solid (1.25 g, 86% yield). ¹H NMR (500 MHZ, CDCl₃) δ 9.07 (d, J=11.7 Hz, 1H), 8.29 (d, J=7.7 Hz, 1H), 7.96 (ddd, J=7.7, 7.7, 1.4 Hz, 1H), 7.86-7.74 (series of br m, 3H), 7.64 (br d, J=7.7 Hz, 1H), 7.58-7.48 (series of br m, 2H), 7.42 (d, J=8.5 Hz, 1H), 7.35 (dd, J=7.8, 7.8 Hz, 1H), 7.24 (dd, J=7.6 Hz, 1H), 7.10 (dd, J=11.6, 8.5 Hz, 1H); ¹³C NMR (125 MHz, CDCl₃) ppm 163.5 (d, ³J_{C-F}=3.8 Hz), 160.8 (d, ¹J_{C-F}=249 Hz), 160.2, 154.4, 146.8, 135.3, 135.0, 134.9 (d, ³J_{C-F}=9.3 Hz), 134.2, 132.2 (d, ⁴J_{C-F}=1.9 Hz), 130.5, 129.5, 128.7, 128.1, 127.6, 127.2, 126.6, 125.0 (d, ³J_{C-F}=3.2 Hz), 121.0, 117.9 (d, ²J_{C-F}=12.5 Hz), 116.3 (d, ²J_{C-F}=24.0 Hz); 19F NMR (470 MHZ, CDCl₃) δ −110.0; HRMS (ESI) m/z: [M+H]⁺ calcd for C₂₁H₁₄ClFN₃O₂ 395.0753 found 395.0756.

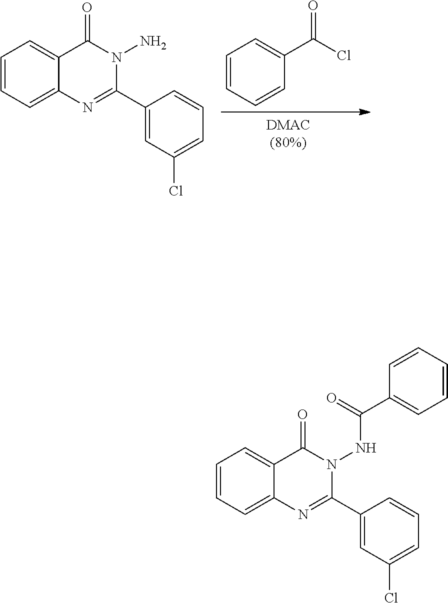

N-(2-(3-Chlorophenyl)-4-oxoquinazolin-3(4H)-yl)benzamide (S55/BZQ). Following General Procedure A, the amine (893 mg, 3.29 mmol) and acid chloride (459 μL, 3.95 mmol) in dimethylacetamide (33 mL) after filtration afforded the product as a colorless solid (991 mg, 80% yield). ¹H NMR (500 MHZ, DMSO-d₆) δ 11.79 (s, 1H), 8.23 (d, J=8.0 Hz, 1H), 8.00-7.91 (m, 1H), 7.83 (d, J=8.0 Hz, 1H), 7.78 (br s, 1H), 7.73 (d, J=7.3 Hz, 2H), 7.71-7.47 (series of br m, 7H); ¹³C NMR (125 MHz, DMSO-d₆) ppm 165.7, 159.5, 154.9, 146.5, 135.6, 135.2, 132.8, 132.6, 131.1, 130.3, 130.0, 128.9, 128.3, 128.0, 127.9, 127.5, 127.3, 126.7, 120.9; HRMS (ESI) m/z: [M+H]⁺ calcd for C₂₁H₁₅ClN₃O₂ 376.0847; found 376.0849.

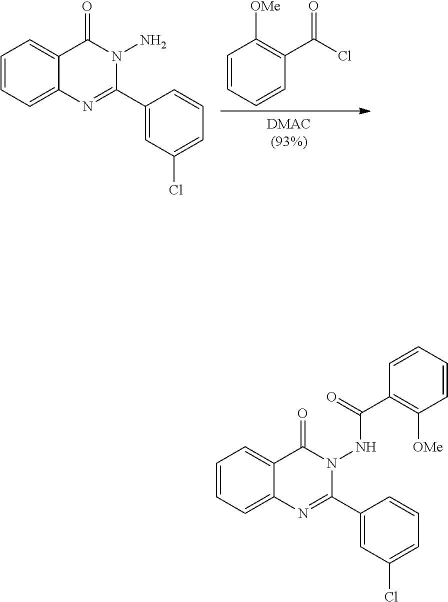

N-(2-(3-Chlorophenyl)-4-oxoquinazolin-3(4H)-yl)-2-methoxybenzamide. Following General Procedure A, the amine (125 mg, 460 μmol) and acid chloride (82 μL, 552 μmol) in dimethylacetamide (4.6 mL) after filtration afforded the product as a colorless solid (174 mg, 93% yield). ¹H NMR (500 MHz, DMSO-d₆) δ 11.08 (s, 1H), 8.22 (dd, J=7.1, 0.8 Hz, 1H), 7.98-7.91 (m, 1H), 7.85 (br s, 1H), 7.81 (d, J=8.1 Hz, 1H), 7.72 (d, J=7.7 Hz, 1H), 7.66 (br dd, J=7.7, 7.7 Hz, 1H), 7.62 (dd, J=7.7, 1.6 Hz, 1H), 7.58-7.47 (series of br m, 3H), 7.17 (d, J=8.4 Hz, 1H), 7.03 (dd, J=7.5, 7.5 Hz, 1H), 3.89 (s, 3H); ¹³C NMR (125 MHZ, DMSO-d₆) ppm 164.4, 159.2, 157.5, 155.1, 146.5, 135.5, 135.4, 134.0, 132.4, 130.7, 130.1, 129.9, 128.6, 127.9, 127.6, 126.7, 121.0, 120.7, 119.6, 112.4, 56.1 (One methine signal is not observed).

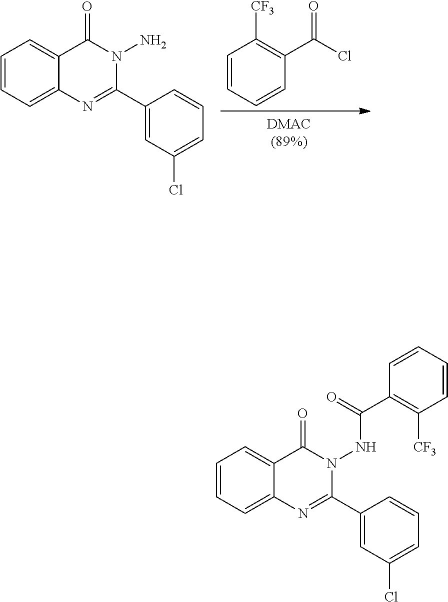

N-(2-(3-Chlorophenyl)-4-oxoquinazolin-3(4H)-yl)-2-(trifluoromethyl)benzamide. Following General Procedure A, the amine (75 mg, 276 μmol) and acid chloride (45 μL, 304 μmol) in dimethylacetamide (3.0 mL) after filtration afforded the product as a colorless solid (109 mg, 89% yield). ¹H NMR (500 MHZ, DMSO-d₆) δ 11.90 (s, 1H), 8.26 (d, J=7.4 Hz, 1H), 7.97 (br dd, J=7.1, 7.1 Hz, 1H), 7.89-7.60 (series of br m, 8H), 7.56 (dd, J=7.9 Hz, 1H), 7.45 (d, J=7.4 Hz, 1H); ¹³C NMR (125 MHz, DMSO-d₆) ppm 165.7, 159.3, 154.7, 146.4, 135.5, 135.0, 132.63, 132.58, 132.1, 131.4, 130.1, 130.0, 128.9, 128.2, 127.89, 127.86, 127.3, 126.8 (q, ³J_{C-F}=3.9 Hz), 126.7, 126.5, 123.0 (q, ¹J_{C-F}=272 Hz), 120.9; ¹⁹F NMR (470 MHZ, DMSO-d₆) δ −58.2.

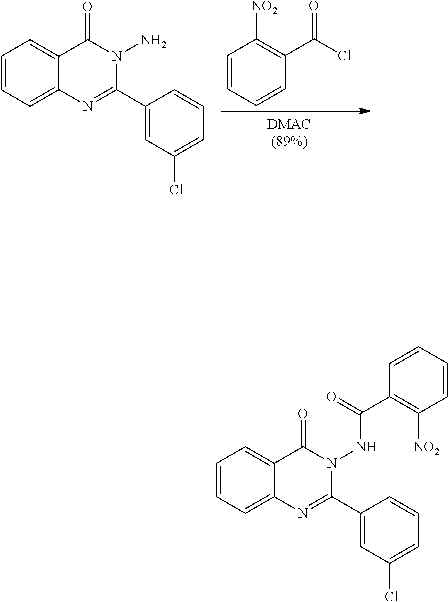

N-(2-(3-Chlorophenyl)-4-oxoquinazolin-3(4H)-yl)-2-nitrobenzamide. Following General Procedure A, the amine (75.0 mg, 276 μmol) and acid chloride (61.4 mg, 331 μmol) in dimethylacetamide (2.8 mL), after filtration, afforded the amide as a colorless solid (103 mg, 89% yield). ¹H NMR (500 MHZ, DMSO-d₆) δ 12.09 (s, 1H), 8.26 (dd, J=7.9, 1.0 Hz, 1H), 8.07 (d, J=8.0 Hz, 1H), 7.97 (ddd, J=8.3, 8.3, 1.4 Hz, 1H), 7.87 (ddd, J=7.5, 7.5, 0.7 Hz, 1H), 7.82 (d, J=8.1 Hz, 1H), 7.79 (ddd. J=7.5, 7.5, 1.2 Hz, 1H), 7.76-7.72 (br m, 1H), 7.70-7.62 (series of br m, 3H), 7.57 (dd, J=7.9, 7.9 Hz, 1H), 7.51 (dd, J=7.5, 1.0 Hz, 1H); ¹³C NMR (125 MHz, DMSO-d₆) ppm 164.4, 159.3, 154.6, 147.2, 146.4, 135.5, 135.0, 133.7, 132.7, 132.4, 130.3, 130.0, 129.1, 128.5, 128.3, 127.92, 127.89, 127.3, 126.7, 124.6, 120.9; HRMS (ESI) m/z: [M+H]⁺ calcd for C₂₁H₁₄ClN₄O₄ 421.0698 found 421.0700.

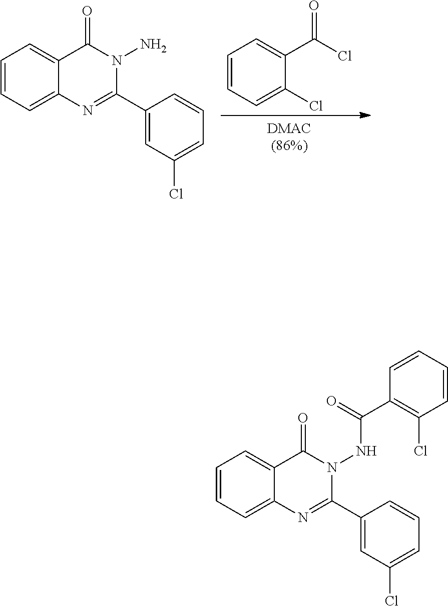

2-Chloro-N-(2-(3-chlorophenyl)-4-oxoquinazolin-3(4H)-yl)benzamide. Following General Procedure A, the amine (75.0 mg, 276 μmol) and acid chloride (42.0 μL, 331 μmol) in dimethylacetamide (2.8 mL), after filtration, afforded the amide as a colorless solid (97.2 mg, 86% yield). ¹H NMR (500 MHZ, DMSO-d₆) δ 11.78 (s, 1H), 8.26 (dd, J=7.9, 1.0 Hz, 1H), 7.97 (ddd, J=8.3, 8.3, 1.4 Hz, 1H), 7.82 (d, J=8.0 Hz, 1H), 7.73 (br s, 1H), 7.70-7.61 (series of br m, 3H), 7.59-7.50 (series of br m, 3H), 7.49-7.41 (m, 1H), 7.31 (d, J=7.5 Hz, 1H); ¹³C NMR (125 MHZ, DMSO-d₆) ppm 165.2, 159.1, 154.7, 146.4, 135.5, 135.1, 133.0, 132.7, 132.3, 130.4, 130.2, 130.1, 129.0, 128.2, 128.0, 127.9, 127.32, 127.25, 126.7, 120.9;Error! Bookmark not defined. HRMS (ESI) m/z: [M+H]⁺ calcd for C₂₁H₁₄Cl₂N₃O₂ 410.0458 found 410.0460.

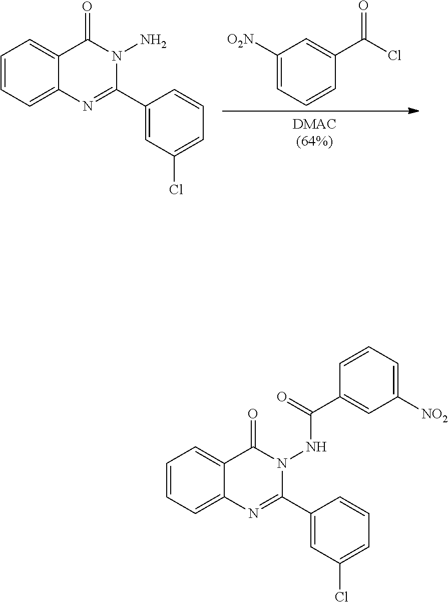 [SMILES: Nn1c(-c2cccc(Cl)c2)nc2ccccc2c1=O ; O=C(Cl)c1cccc([N+](=O)[O-])c1 ; O=C(Nn1c(-c2cccc(Cl)c2)nc2ccccc2c1=O)c1cccc([N+](=O)[O-])c1]

N-(2-(3-Chlorophenyl)-4-oxoquinazolin-3(4H)-yl)-3-nitrobenzamide. Following General Procedure A, the amine (75.0 mg, 276 μmol) and acid chloride (61.4 mg, 331 μmol) in dimethylacetamide (2.8 mL), after filtration, afforded the amide as a colorless solid (74.0 mg, 64% yield). ¹H NMR (500 MHz, DMSO-d₆) δ 12.24 (s, 1H), 8.55 (br s, 1H), 8.47 (dd, J=8.2, 1.5 Hz, 1H), 8.24 (d, J=7.2 Hz, 1H), 8.17 (d, J=7.8 Hz, 1H), 7.98 (br ddd, J=7.7, 7.7, 1.2 Hz, 1H), 7.88-7.81 (series of m, 2H), 7.79 (br s, 1H), 7.71-7.61 (m, 2H), 7.58 (br d, J=8.5 Hz, 1H), 7.51 (dd, J=7.8, 7.8 Hz, 1H); ¹³C NMR (125 MHZ, DMSO-d₆) ppm 164.2, 159.7, 154.9, 148.3, 146.8, 136.0, 135.4, 134.2, 133.1, 132.6, 131.3, 130.7, 130.5, 128.7, 128.42, 128.35, 127.8, 127.5, 127.1, 122.5, 121.3.

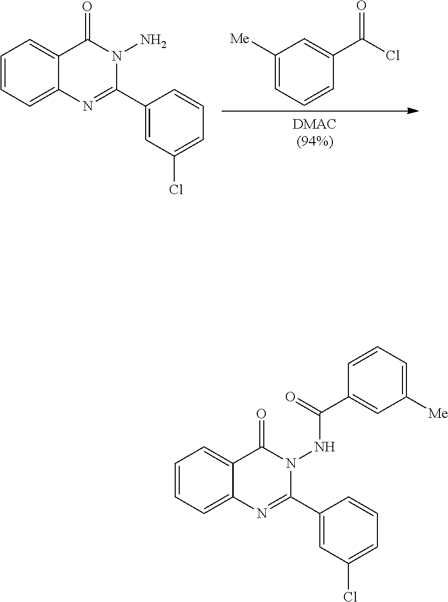 [SMILES: Nn1c(-c2cccc(Cl)c2)nc2ccccc2c1=O ; Cc1cccc(C(=O)Cl)c1 ; Cc1cccc(C(=O)Nn2c(-c3cccc(Cl)c3)nc3ccccc3c2=O)c1]

N-(2-(3-Chlorophenyl)-4-oxoquinazolin-3(4H)-yl)-3-methylbenzamide. Following General Procedure A, the amine (75.0 mg, 276 μmol) and acid chloride (40.0 μL, 304 μmol) in dimethylacetamide (3.0 mL), after filtration, afforded the amide as a colorless solid (101 mg, 94% yield). ¹H NMR (500 MHZ, DMSO-d₆) δ11.72 (s, 1H), 8.23 (d, J=7.9 Hz, 1H), 7.96 (ddd, J=7.6, 7.6, 1.2 Hz, 1H), 7.83 (d, J=8.1 Hz, 1H), 7.79 (br s, 1H), 7.72-7.63 (series of m, 2H), 7.61-7.48 (series of m, 4H), 7.42 (dd, J=7.5, 7.5 Hz, 1H), 7.39 (dd, J=7.5, 7.5 Hz, 1H), 2.35 (s, 3H); ¹³C NMR (125 MHZ, DMSO-d₆) ppm 166.1, 159.8, 155.2, 146.9, 138.6, 135.9, 135.6, 133.7, 133.0, 131.5, 130.6, 130.4, 129.1, 128.7, 128.4, 128.32, 128.29, 127.6, 127.0, 124.9, 121.3, 21.3.

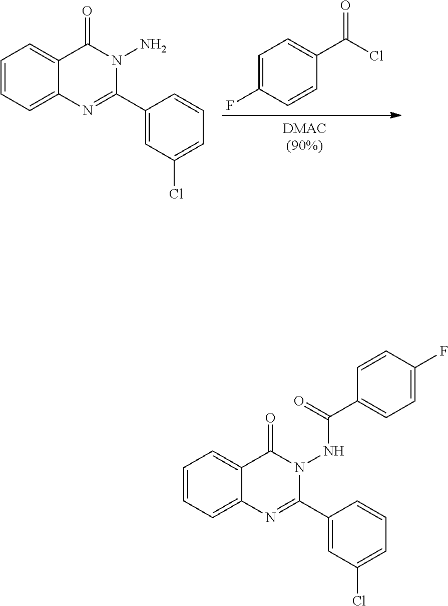

N-(2-(3-Chlorophenyl)-4-oxoquinazolin-3(4H)-yl)-4-fluorobenzamide. Following General Procedure A, the amine (125 mg, 460 μmol) and acid chloride (65 μL, 552 μmol) in dimethylacetamide (4.6 mL) after filtration afforded the product as a colorless solid (163 mg, 90% yield). ¹H NMR (500 MHz, DMSO-d₆) δ11.84 (s, 1H), 8.22 (br d, J=7.2 Hz, 1H), 8.00-7.93 (br m, 1H), 7.89-7.78 (series of br m, 3H), 7.77 (br s, 1H), 7.67 (br dd, J=7.8, 7.8 Hz, 2H), 7.57 (br d, J=8.1 Hz, 1H), 7.51 (dd, J=7.8, 7.8 Hz, 1H), 7.37 (dd, J=8.8, 8.8 Hz, 2H); ¹³C NMR (125 MHz, DMSO-d₆) ppm 164.7 (d, 1J_{C-F}=249 Hz), 164.6, 159.5, 154.8, 146.5, 135.6, 135.2, 132.7, 130.3, 130.2 (d, 2J_{C-F}=39 Hz), 128.3, 128.0 (d, 3J_{C-F}=5.6 Hz), 127.5 (d, ⁴J_{C-F}=3.0 Hz), 127.2, 126.7, 120.9, 116.1, 115.9; Error! Bookmark not defined. ¹⁹F NMR (470 MHZ, DMSO-d₆) δ −106.5.

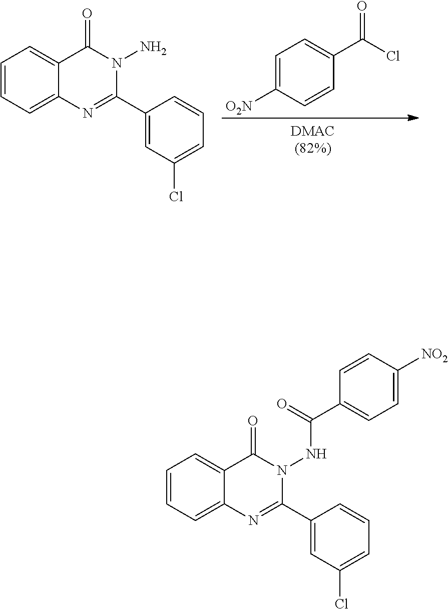

N-(2-(3-Chlorophenyl)-4-oxoquinazolin-3(4H)-yl)-4-nitrobenzamide. Following General Procedure A, the amine (75.0 mg, 276 μmol) and acid chloride (56.4 mg, 304 μmol) in dimethylacetamide (3.0 mL), after filtration, afforded the amide as a colorless solid (95.6 mg, 82% yield). ¹H NMR (500 MHZ, DMSO-d₆) δ 12.20 (s, 1H), 8.37 (d, J=8.8 Hz, 2H), 8.24 (br d, J=7.0 Hz, 1H), 7.98 (ddd, J=8.4, 8.4, 1.2 Hz, 1H), 7.94 (d, J=8.8 Hz, 2H), 7.84 (d, J=8.1 Hz, 1H), 7.77 (br s, 1H), 7.71-7.65 (series of br m, 2H), 7.59 (br d, J=8.8 Hz, 1H), 7.52 (dd, J=7.9, 7.9 Hz, 1H); ¹³C NMR (125 MHz, DMSO-d₆) ppm 164.2, 159.3, 154.5, 149.9, 146.4, 136.4, 135.6, 135.0, 132.7, 130.3, 130.0, 129.0, 128.2, 128.0, 127.9, 127.1, 126.7, 124.1, 120.8; HRMS (ESI) m/z: [M+H]⁺ calcd for C₂₁H₁₄ClN₄O₄ 421.0698 found 421.0699.

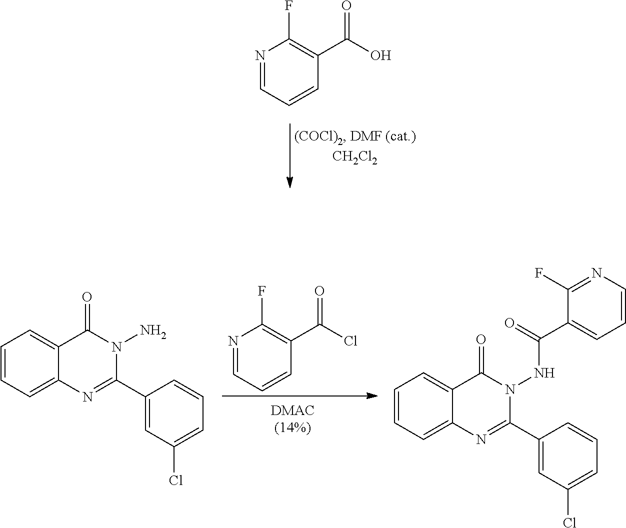

N-(2-(3-Chlorophenyl)-4-oxoquinazolin-3(4H)-yl)-2-fluoronicotinamide. To a solution of the acid (250 mg, 1.77 mmol) and DMF (5.5 μL) in CH₂Cl₂ (20 mL), chilled to 0° C. in an ice bath, was added oxalyl chloride (2 M in CH₂Cl₂, 1.07 mL, 2.13 mmol) dropwise. The ice bath was removed, and the reaction was stirred for 3 h and then concentrated in vacuo. Excess HCl was removed via concentrating from CH₂Cl₂ (5×). The resulting acid chloride was used without further manipulation.

The acid chloride was dissolved in N,N-dimethylacetamide (15 mL), and the solution was chilled to 0° C. in an ice bath. The amine was added, and the solution was transferred to a heating mantle and stirred at 80° C. for 16 h. The reaction was concentrated in vacuo, and the residue was dissolved in 1:1 CH₂Cl₂/sat aq NaHCO₃ and separated. The aq layer was extracted with CH₂Cl₂, and the combined organic layers were washed with sat aq NaHCO₃ and brine, and then dried and concentrated. The residue was dissolved in CH₂Cl₂ and dry loaded onto Celite. MPLC (Sfar KP-NH, 28 g, 25-100% ethyl acetate in hexanes) afforded the product as a colorless amorphous solid (78.9 mg, 14% yield). ¹H NMR (500 MHZ, DMSO-d₆) δ11.89 (br s, 1H), 8.44 (br d, J=3.6 Hz, 1H), 8.24 (d, J=7.0 Hz, 1H), 8.07-7.93 (series of m, 2H), 7.83 (d, J=8.1 Hz, 1H), 7.76 (br s, 1H), 7.71-7.61 (series of m, 3H), 7.57-7.49 (series of m, 2H); ¹³C NMR (125 MHZ, DMSO-d₆) ppm 162.1 (d, ³J_{C-F}=6.1 Hz), 159.2 (d, 1J_{C-F}=240 Hz), 159.1, 154.5, 151.1 (d, ³J_{C-F}=14.8 Hz), 146.4, 141.6 (d, ⁴J_{C-F}=2.8 Hz), 135.6, 135.0, 132.7, 130.4, 130.1, 128.3, 128.1, 128.0, 127.2, 126.7, 122.6 (d, ⁴J_{C-F}=4.4 Hz), 120.9, 115.2 (d, 2J_{C-F}=30 HZ).

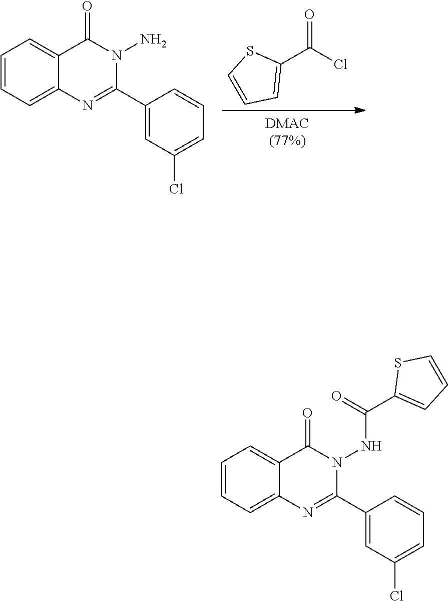

N-(2-(3-Chlorophenyl)-4-oxoquinazolin-3(4H)-yl)thiophene-2-carboxamide. Following General Procedure A, the amine (75 mg, 276 μmol) and acid chloride (35.4 μL, 331 μmol) in dimethylacetamide (3.0 mL) after filtration afforded the product as a colorless solid (81.1 mg, 77% yield). ¹H NMR (500 MHZ, DMSO-d₆) δ11.79 (s, 1H), 8.23 (d, J=7.3 Hz, 1H), 7.97 (ddd, J=7.1, 7.1, 1.1 Hz, 1H), 7.92 (d, J=4.8 Hz, 1H), 7.83 (br d, J=7.5 Hz, 1H), 7.81 (d, J=4.0 Hz, 1H), 7.77 (br s, 1H), 7.66 (m, 2H), 7.56 (br d, J=8.2 Hz, 1H), 7.50 (dd, J=7.8 Hz, 1H), 7.22 (dd, J=4.1 Hz, 1H); ¹³C NMR (125 MHZ, DMSO-d₆) ppm 160.5, 159.6, 154.8, 146.5, 135.6, 135.2, 135.0, 133.2, 132.6, 130.5, 130.3, 130.1, 128.5, 128.4, 128.01, 127.96, 127.3, 126.7, 120.9.

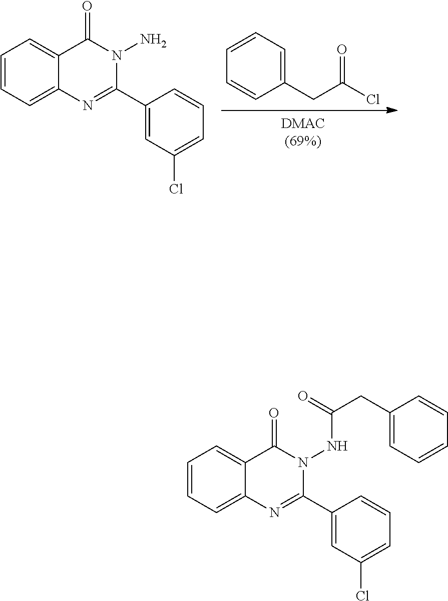

N-(2-(3-Chlorophenyl)-4-oxoquinazolin-3(4H)-yl)-2-phenylacetamide. Following General Procedure A, the amine (75 mg, 276 μmol) and acid chloride (44 μL, 331 μmol) in dimethylacetamide (3.0 mL) after filtration afforded the product as a colorless solid (74.1 mg, 69% yield). ¹H NMR (500 MHZ, DMSO-d₆) § 11.44 (ds, 1H), 8.20 (d, J=7.4 Hz, 1H), 7.96-7.89 (m, 1H), 7.77 (J=8.1 Hz, 1H), 7.63 (d, J=7.7 Hz, 1H), 7.61 (br s, 1H), 7.57 (br d, J=7.8 Hz, 1H), 7.42 (dd, J=7.8, 7.8 Hz, 1H), 7.25-7.13 (m, 3H), 6.99-6.90 (m, 2H), 3.52 (d, J=14.5 Hz, 1H), 3.47 (d, J=14.5 Hz, 1H); ¹³C NMR (125 MHZ, DMSO-d₆) ppm 169.4, 159.3, 154.9, 146.5, 135.4, 135.2, 134.4, 132.9, 130.0, 129.9, 128.7, 128.3, 128.2, 127.8, 127.1, 126.7, 126.6, 121.0.

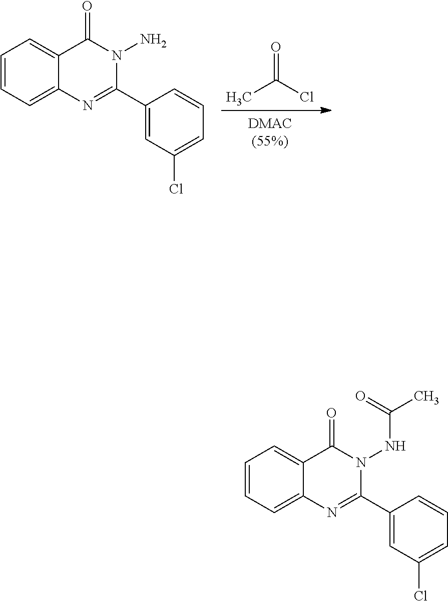

N-(2-(3-Chlorophenyl)-4-oxoquinazolin-3(4H)-yl) acetamide. To a solution of the amine (75 mg, 276 μmol) in dimethylacetamide (3.0 mL) was added acetyl chloride (1.0 in CH₂Cl₂, 359 μL, 359 μmol), and the reaction was stirred at ambient temperature for 24 h. The reaction was poured into H₂O and allowed to stand at ambient temperature for 24 h. Tan crystals formed and were collected by filtration, washed with H₂O, and dried in vacuo to afford the product as a tan solid (47.7 mg, 55% yield). ¹H NMR (500 MHZ, DMSO-d₆) δ 11.15 (s, 1H), 8.19 (d, J=7.1 Hz, 1H), 7.96-7.89 (br m, 1H), 7.78 (d, J=8.1 Hz, 1H), 7.68-7.50 (series of br m, 5H), 1.85 (s, 3H); ¹³C NMR (125 MHz, DMSO-d₆) ppm 168.9, 159.3, 154.9, 146.5, 135.41, 135.39, 132.6, 130.1, 130.0, 128.2, 127.82, 127.79, 127.1, 126.6, 121.0, 20.3.

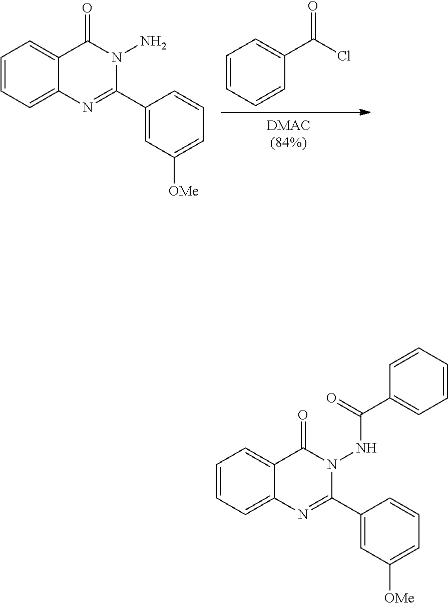

N-(2-(3-Methoxyphenyl)-4-oxoquinazolin-3(4H)-yl)benzamide. Following General Procedure A, the amine (100.0 mg, 374 μmol) and acid chloride (48.0 μL, 445 μmol) in dimethylacetamide (3.3 mL), after filtration, afforded the amide as a colorless solid (104 mg, 84% yield). ¹H NMR (500 MHz, DMSO-d₆) δ 11.72 (s, 1H), 8.23 (br dd, J=7.9, 0.85 Hz, 1H), 7.95 (ddd, J=8.2, 1.3, 1.3 Hz, 1H), 7.81 (d, J=8.0 Hz, 1H), 7.74 (d, J=7.3 Hz, 2H), 7.65 (dd, J=7.7, 7.7 Hz, 1H), 7.61 (dd, J=7.4, 7.4 Hz, 1H), 7.51 (dd, J=7.7, 7.7 Hz, 2H), 7.38 (dd, J=7.9, 7.9 Hz, 1H), 7.32-7.24 (series of br m, 2H), 7.05 (dd, J=8.2, 2.2 Hz, 1H), 3.77 (s, 3H); ¹³C NMR (125 MHz, DMSO-d₆) ppm 165.4, 159.6, 158.5, 156.0, 146.6, 135.4, 134.6, 132.7, 131.2, 129.2, 128.7, 127.8, 127.6, 127.4, 126.6, 120.78, 120.76, 116.1, 113.8, 55.3; HRMS (ESI) m/z: [M+H]⁺ calcd for C₂₂H₁₈N₃O₃ 372.1343 found 372.1344.

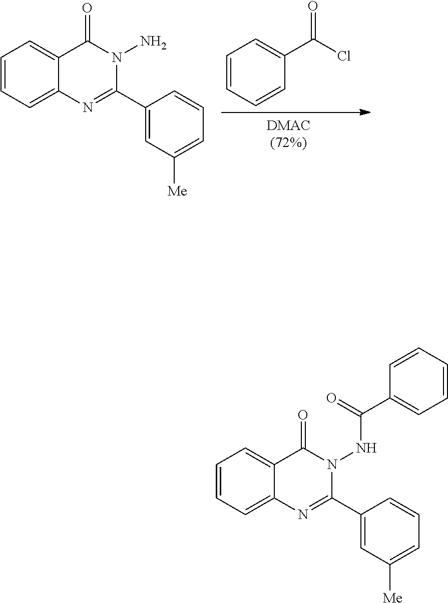

N-(4-Oxo-2-(m-tolyl)quinazolin-3(4H)-yl)benzamide. Following General Procedure A, the amine (100.0 mg, 398 μmol) and acid chloride (56.0 μL, 478 μmol) in dimethylacetamide (4.0 mL), after filtration, afforded the amide as a colorless solid (102 mg, 72% yield). ¹H NMR (500 MHZ, DMSO-d₆) δ 11.72 (s, 1H), 8.22 (dd, J=8.0, 1.0 Hz, 1H), 7.95 (ddd, J=8.3, 8.3, 1.4 Hz, 1H), 7.81 (d, J=8.2 Hz, 1H), 7.72 (d, J=7.3 Hz, 2H), 7.69-7.47 (series of br m, 6H), 7.35 (dd, J=7.6, 7.6 Hz, 1H), 7.30 (br d, J=7.6 Hz, 1H), 2.34 (s, 3H); ¹³C NMR (125 MHz, DMSO-d₆) ppm 165.4, 159.6, 156.4, 146.7, 137.2, 135.4, 133.3, 132.6, 131.3, 130.8, 129.1, 128.7, 127.8, 127.7, 127.5, 127.4, 126.6, 125.6, 120.7, 20.9; HRMS (ESI) m/z: [M+H]⁺ calcd for C₂₂H₁₈N₃O₂ 356.1394 found 356.1395.

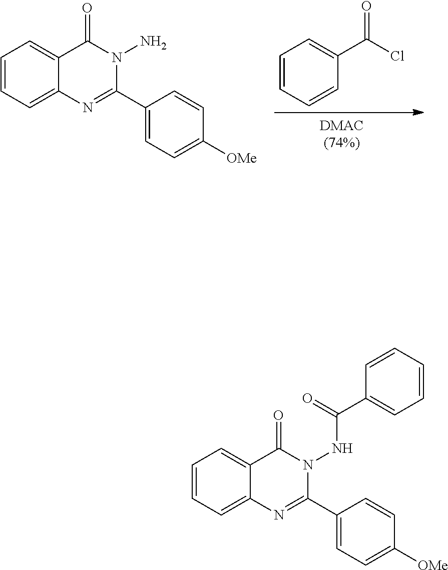 [SMILES: COc1ccc(-c2nc3ccccc3c(=O)n2N)cc1 ; O=C(Cl)c1ccccc1 ; COc1ccc(-c2nc3ccccc3c(=O)n2NC(=O)c2ccccc2)cc1]

N-(2-(4-Methoxyphenyl)-4-oxoquinazolin-3(4H)-yl)benzamide. Following General Procedure A, the amine (100.0 mg, 374 μmol) and acid chloride (53.0 μL, 449 μmol) in dimethylacetamide (3.7 mL), after filtration, afforded the amide as a colorless solid (91.6 mg, 74% yield). ¹H NMR (500 MHZ, DMSO-d₆) δ11.74 (s, 1H), 8.20 (br dd, J=8.0, 1.0 Hz, 1H), 7.93 (br ddd, J=8.3, 8.3, 1.4 Hz, 1H), 7.79 (d, J=8.4 Hz, 1H), 7.78 (d, J=7.3 Hz, 2H), 7.74 (d, J=8.8 Hz, 2H), 7.65-7.58 (series of m, 2H), 7.52 (dd, J=7.8, 7.8 Hz, 2H), 7.02 (d, J=8.8 Hz, 2H), 3.78 (s, 3H); ¹³C NMR (125 MHz, DMSO-d₆) ppm 165.4, 160.7, 159.8, 156.0, 146.8, 135.3, 132.6, 131.2, 130.5, 128.8, 127.7, 127.5, 127.3, 126.6, 125.5, 120.5, 113.3, 55.3; HRMS (ESI) m/z: [M+H]⁺ calcd for C₂₂H₁₈N₃O₃ 372.1343 found 372.1345.

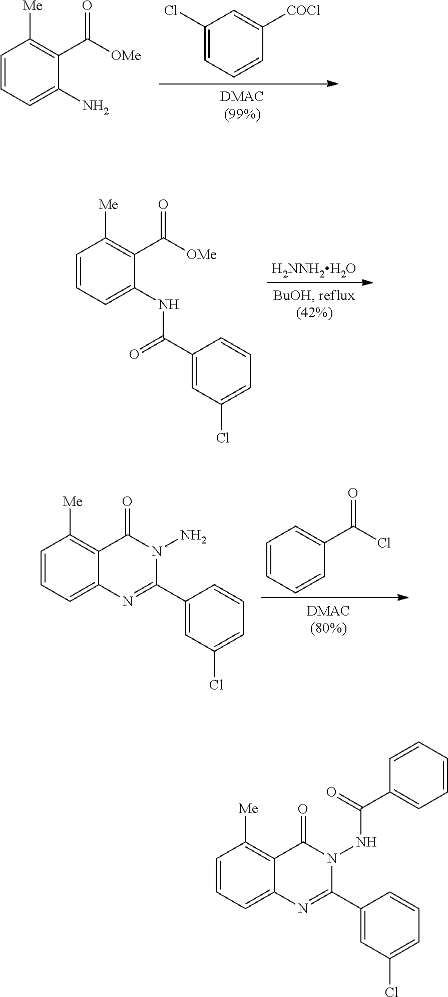

N-(2-(3-Chlorophenyl)-5-methyl-4-oxoquinazolin-3(4H)-yl)benzamide. Following General Procedure A, the amine (500 mg, 3.03 mmol) and acid chloride (466 μL, 3.64 mmol) in N,N-dimethylacetamide (30 mL) after filtration afforded the product as a colorless solid (910 mg, 99% yield).

A suspension of the ester (710 mg, 2.34 mmol) in H₂NNH₂·H₂O (7.8 mL) and 1-butanol (11.7 mL) was refluxed in a heating mantle for 18 h. After cooling to ambient temperature, the reaction was poured into H₂O and stirred vigorously for 30 minutes. The precipitate was filtered and dried in vacuo to afford the product as a free-flowing, colorless solid (284 mg, 42% yield).

Following General Procedure A, the amine (100.0 mg, 350 μmol) and acid chloride (49.0 μL, 420 μmol) in dimethylacetamide (3.5 mL), after filtration, afforded the amide as a colorless solid (109 mg, 80% yield). ¹H NMR (500 MHZ, DMSO-d₆) δ 11.64 (s, 1H), 7.83-7.70 (series of m, 4H), 7.68 (br d, J=7.7 Hz, 1H), 7.65-7.58 (series of m, 2H), 7.58-7.47 (series of m, 4H), 7.42 (d, J=7.4 Hz, 1H), 2.80 (s, 3H); ¹³C NMR (125 MHZ, DMSO-d₆) ppm 165.7, 159.7, 154.5, 147.9, 140.7, 135.2, 134.5, 132.7, 132.5, 131.1, 130.13, 130.09, 129.9, 128.8, 128.2, 127.4, 127.1, 126.1, 119.2, 22.4; HRMS (ESI) m/z: [M+H]⁺ calcd for C₂₂H₁₇ClN₃O₂ 390.1004 found 390.1007.

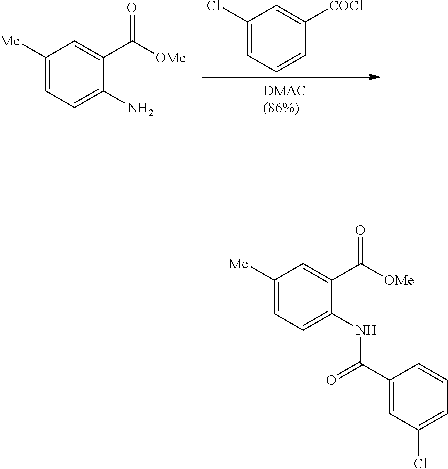 [SMILES: COC(=O)c1cc(C)ccc1N ; O=C(Cl)c1cccc(Cl)c1 ; COC(=O)c1cc(C)ccc1NC(=O)c1cccc(Cl)c1]

Methyl 2-(3-chlorobenzamido)-5-methylbenzoate. Following General Procedure A, the amine (1.00 g, 6.05 mmol) and acid chloride (929 μL, 7.26 mmol) in N,N-dimethylacetamide (61 mL) after filtration afforded the product as a colorless solid (1.58 g, 86% yield). ¹H NMR (500 MHZ, CDCl₃) δ 11.94 (s, 1H), 8.77 (d, J=8.6 Hz, 1H), 8.04 (br s, 1H), 7.92-7.86 (br m, 2H), 7.52 (br d, J=8.8 Hz, 1H), 7.48-7.39 (series of br m, 2H), 3.96 (s, 3H), 2.36 (s, 3H); ¹³C NMR (125 MHz, CDCl₃) ppm 169.1, 164.1, 139.1, 136.8, 135.6, 135.0, 132.5, 131.8, 131.1, 130.0, 128.0, 125.0, 120.4, 115.1, 52.5, 20.7.

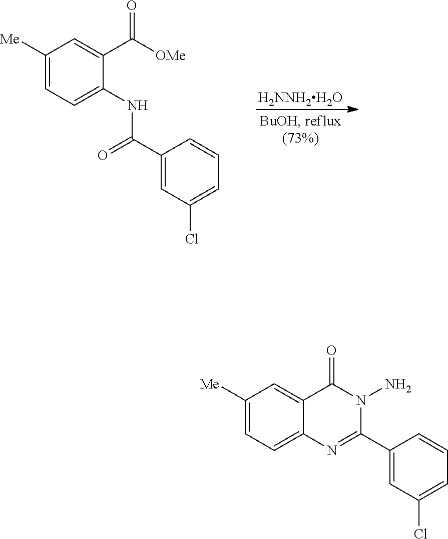 [SMILES: COC(=O)c1cc(C)ccc1NC(=O)c1cccc(Cl)c1 ; "H2NNH2·H2O" ; Cc1ccc2nc(-c3cccc(Cl)c3)n(N)c(=O)c2c1]

3-Amino-2-(3-chlorophenyl)-6-methylquinazolin-4 (3H)-one. A suspension of the ester (600 mg, 1.98 mmol) in H₂NNH₂·H₂O (6.6 mL) and 1-butanol (13 mL) was refluxed in a heating mantle for 18 h. After cooling to ambient temperature, the reaction was poured into H₂O and stirred vigorously for 30 minutes. The precipitate was filtered and dried in vacuo to afford the product as a free-flowing, colorless solid (411 mg, 73% yield). ¹H NMR (500 MHz, DMSO-d₆) δ 7.99 (s, 1H), 7.86 (s, 1H), 7.75 (br d, J=7.6 Hz, 1H), 7.68 (br d, J=7.6 Hz, 1H), 7.63 (d, J=8.3 Hz, 1H), 7.56 (br d, J=8.1 Hz, 1H), 7.51 (dd, J=7.9 Hz, 1H), 5.65 (s, 2H), 2.48 (s, 3H); ¹³C NMR (125 MHz, DMSO-d₆) ppm 161.5, 154.1, 145.1, 137.3, 137.2, 136.2, 132.5, 129.84, 129.79, 129.67, 128.7, 127.8, 125.7, 120.4, 21.3.

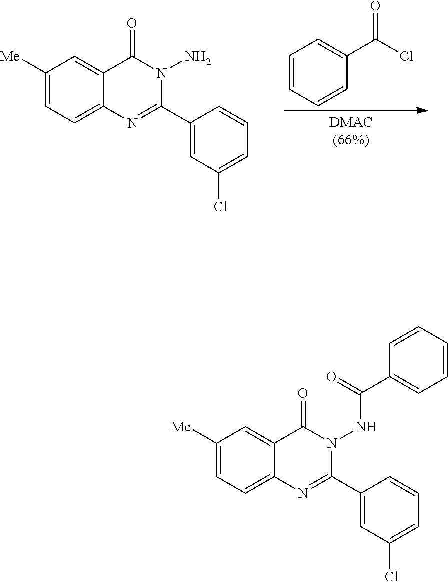 [SMILES: Cc1ccc2nc(-c3cccc(Cl)c3)n(N)c(=O)c2c1 ; O=C(Cl)c1ccccc1 ; Cc1ccc2nc(-c3cccc(Cl)c3)n(NC(=O)c3ccccc3)c(=O)c2c1]

N-(2-(3-Chlorophenyl)-6-methyl-4-oxoquinazolin-3(4H)-yl)benzamide. Following General Procedure A, the amine (100.0 mg, 350 μmol) and acid chloride (49.0 μL, 420 μmol) in dimethylacetamide (3.5 mL), after filtration, afforded the amide as a colorless solid (109 mg, 80% yield). ¹H NMR (500 MHZ, DMSO-d₆) δ 11.75 (s, 1H), 8.02 (br s, 1H), 7.82-7.43 (series of br m, 11H), 2.49 (s, 3H); ¹³C NMR (125 MHz, DMSO-d₆) 165.9, 159.8, 154.4, 144.9, 138.3, 137.1, 135.7, 133.1, 133.0, 131.5, 130.5, 130.3, 129.7, 129.2, 129.0, 128.7, 128.2, 127.8, 127.6, 126.3, 121.1, 21.3.

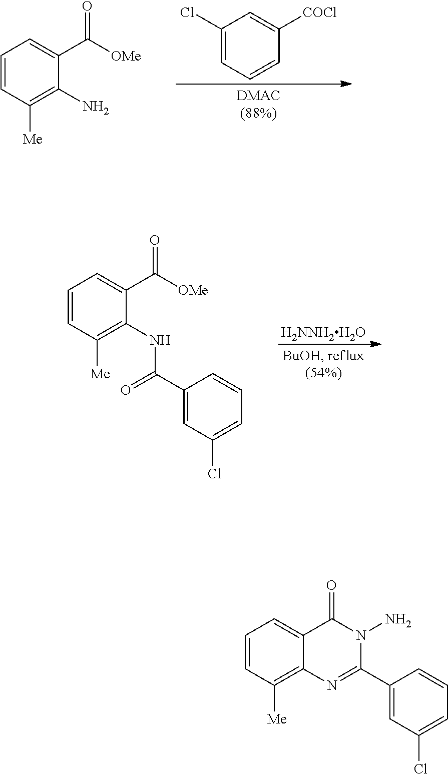 [SMILES: COC(=O)c1cccc(C)c1N ; O=C(Cl)c1cccc(Cl)c1 ; COC(=O)c1cccc(C)c1NC(=O)c1cccc(Cl)c1 ; "H2NNH2·H2O" ; Cc1cccc2c(=O)n(N)c(-c3cccc(Cl)c3)nc12]

3-Amino-2-(3-chlorophenyl)-8-methylquinazolin-4 (3H)-one. Following General Procedure A, the amine (1.00 g, 6.05 mmol) and acid chloride (929 μL, 7.29 mmol) in N,N-dimethylacetamide (61 mL) after filtration afforded the amide as a colorless solid (1.62 g, 88% yield). A suspension of the ester (1.18 g, 3.88 mmol) in H₂NNH₂·H₂O (13 mL) and 1-butanol (13 mL) was refluxed in a heating mantle for 18 h. After cooling to ambient temperature, the reaction was poured into H₂O and stirred vigorously for 30 minutes. The precipitate was filtered and dried in vacuo to afford the product as a free-flowing, colorless solid (597 mg, 54% yield). ¹H NMR (500 MHZ, DMSO-d₆) δ 8.03 (d, J=7.9 Hz, 1H), 7.91 (br s, 1H), 7.80 (d, J=7.7 Hz, 1H), 7.71 (d, J=7.2 Hz, 1H), 7.58 (d, J=8.3 Hz, 1H), 7.53 (dd, J=7.8, 7.8 Hz, 1H), 7.46 (dd, J=7.6 Hz, 1H), 5.67 (s, 2H), 2.55 (s, 3H); ¹³C NMR (125 MHZ, DMSO-d₆) ppm 161.3, 153.3, 145.0, 137.1, 135.7, 134.6, 132.1, 129.5, 129.4, 129.3, 128.5, 126.7, 123.7, 120.2, 17.1.

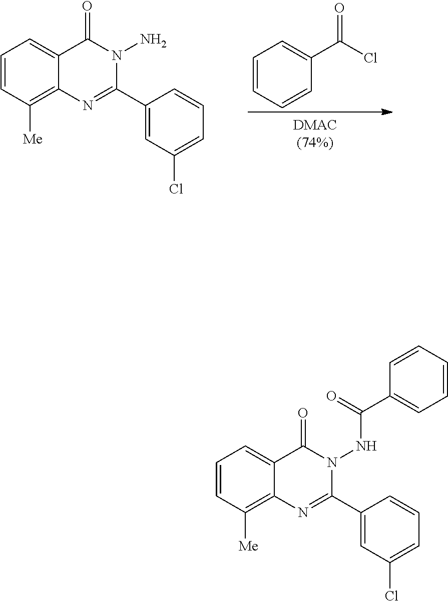 [SMILES: Cc1cccc2c(=O)n(N)c(-c3cccc(Cl)c3)nc12 ; O=C(Cl)c1ccccc1 ; Cc1cccc2c(=O)n(NC(=O)c3ccccc3)c(-c3cccc(Cl)c3)nc12]

N-(2-(3-Chlorophenyl)-8-methyl-4-oxoquinazolin-3(4H)-yl)benzamide. Following General Procedure A, the amine (100.0 mg, 350 μmol) and acid chloride (49.0 μL, 420 μmol) in dimethylacetamide (3.5 mL), after filtration, afforded the amide as a colorless solid (101 mg, 74% yield). ¹H NMR (500 MHz, DMSO-d₆) δ 11.79 (s, 1H), 8.06 (d, J=7.8 Hz, 1H), 7.82 (br d, J=6.7 Hz, 2H), 7.78-7.67 (series of br m, 3H), 7.62 (br dd, J=7.4, 7.4 Hz, 1H), 7.60-7.45 (series of br m, 5H), 2.60 (s, 3H); ¹³C NMR (125 MHz, DMSO-d₆) 165.5, 159.7, 153.6, 144.9, 136.3, 135.8, 135.5, 132.7, 132.6, 131.1, 130.2, 130.0, 128.8, 128.4, 127.5, 127.4, 127.3, 124.3, 120.9, 17.1; HRMS (ESI) m/z: [M+H]⁺ calcd for C₂₂H₁₇ClN₃O₂ 390.1004 found 390.1006.

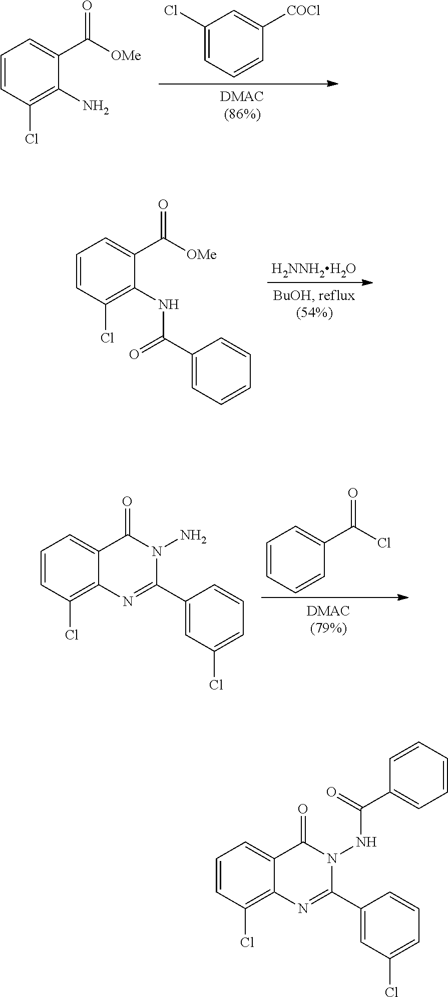

N-(8-Chloro-2-(3-chlorophenyl)-4-oxoquinazolin-3(4H)-yl)benzamide. Following General Procedure A, the amine (1.00 g, 5.39 mmol) and acid chloride (828 μL, 6.47 mmol) in N,N-dimethylacetamide (54 mL) after filtration afforded the ester as a colorless solid (1.51 g, 86% yield).

A suspension of the ester (1.00 g, 3.08 mmol) in H₂NNH₂·H₂O (10 mL) and 1-butanol (10 mL) was refluxed in a heating mantle for 18 h. After cooling to ambient temperature, the reaction was poured into H₂O and stirred vigorously for 30 minutes. The precipitate was filtered and dried in vacuo to afford the product as a free-flowing, colorless solid (479 mg, 54% yield).

Following General Procedure A, the amine (100.0 mg, 327 μmol) and acid chloride (46.0 μL, 392 μmol) in dimethylacetamide (3.3 mL), after filtration, afforded the amide as a colorless solid (106 mg, 79% yield). ¹H NMR (500 MHZ, DMSO-d₆) δ 11.91 (s, 1H), 8.20 (dd, J=7.9, 1.0 Hz, 1H), 8.13 (dd, J=7.9, 1.0 Hz, 1H), 7.95 (br d, J=7.2 Hz, 1H), 7.81 (br s, 1H), 7.78-7.46 (series of m, 8H); ¹³C NMR (125 MHz, DMSO-d₆) 165.5, 159.0, 155.6, 142.9, 135.6, 134.9, 132.8, 132.6, 131.5, 130.9, 130.5, 130.1, 128.8, 128.6, 128.4, 127.5, 127.3, 125.9, 122.7; HRMS (ESI) m/z: [M+H]⁺ calcd for C₂₁H₁₄C₁₂N₃O₂ 410.0458 found 410.0459.

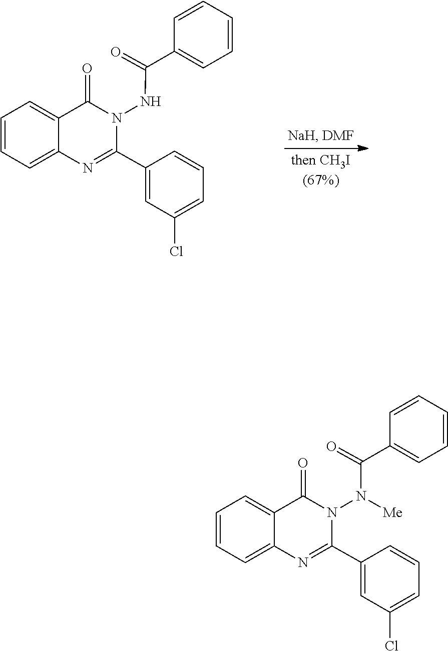 [SMILES: O=C(Nn1c(-c2cccc(Cl)c2)nc2ccccc2c1=O)c1ccccc1 ; CN(C(=O)c1ccccc1)n1c(-c2cccc(Cl)c2)nc2ccccc2c1=O]

N-(2-(3-Chlorophenyl)-4-oxoquinazolin-3(4H)-yl)-N-methylbenzamide (S235). To a suspension of NaH (60% in mineral oil, 24 mg, 599 μmol) in DMF (2 mL), chilled to 0° C. in an ice bath, was added the amide (150 mg, 399 μmol) as a solution in DMF (2 mL). The reaction was stirred for 2 h. and methyl iodide (37.3 μL, 599 μmol) was added. The ice bath was removed, and the reaction was stirred at ambient temperature for 18 h. The reaction was quenched by adding sat aq NH₄Cl and then concentrated in vacuo. The residue was dry loaded onto SiO₂ and purified via MPLC to afford the product as a free-flowing, colorless solid (104 mg, 67% yield).

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

For reasons of completeness, various aspects and embodiments of the present disclosure are set out in the following numbered clauses:

Clause 1. A compound of Formula (I′), or a pharmaceutically acceptable salt thereof:

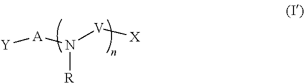

wherein
A is a ring system selected from the group consisting of

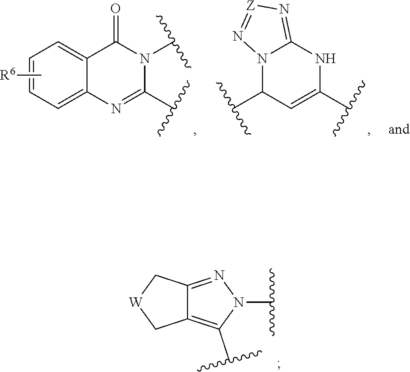

or A is an alkenyl of

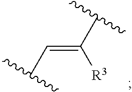

X is alkyl, aryl, heteroaryl, C₁₋₄ alkylenearyl, or cycloalkyl, wherein X is optionally substituted with one or more substituents selected from the group consisting of alkyl, haloalkyl, alkoxy, halogen, cyano, deuterium, and nitro;
Y is alkyl, cycloalkyl, aryl, C₁₋₄ alkylenearyl, or heteroaryl, wherein Y is optionally substituted with one or more substituents selected from the group consisting of halogen, alkoxy optionally substituted with one or more halogen, alkyl, haloalkyl, and nitro;
Z is N or CR⁵;
W is S, S(O), or S(O)₂;
V is C(O) or S(O)₂;
n is 0 or 1; and
   wherein
R is hydrogen, alkyl, aryl, cycloalkyl, or heteroaryl;
R¹ is hydrogen or alkyl;
R² is hydrogen, alkyl optionally substituted with —C(O)OR¹, or aryl optionally substituted with alkyl;
R³ is —OC(O)R′, —C(O)OR′, or —C(O)NR²R⁴;
R⁴ is alkyl optionally substituted with hydroxy;
R⁵ is selected from the group consisting of hydrogen, hydroxyalkyl, —OC(O)R¹, —C(O)OR¹, —NR¹C(O)R², and —NR¹S(O)₂R²; and
R⁶ is an optional substituent selected from the group consisting of halogen, alkyl, haloalkyl, alkoxy, cyano, and nitro,
provided that the compound is not 4-bromo-N-(2-(4-fluorophenyl)-4-oxoquinazolin-3(4H)-yl)benzamide or 4-bromo-N-(2-(2-fluorophenyl)-4-oxoquinazolin-3(4H)-yl)benzamide.

Clause 2. The compound of clause 1, or a pharmaceutically acceptable salt thereof, having a structure of Formula (I),

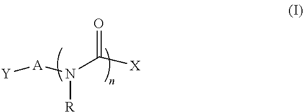

wherein
A is a ring system selected from the group consisting of

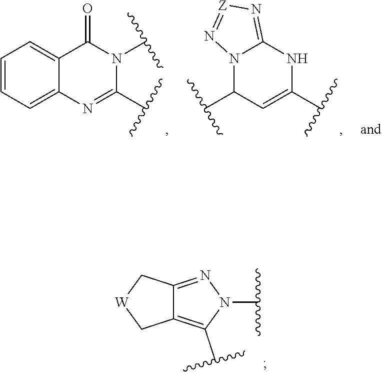

or A is an alkenyl of

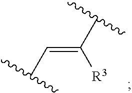

X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
Z is N or CR⁵;
W is S, S(O), or S(O)₂;
n is 0 or 1; and
   wherein
R is hydrogen, alkyl, aryl, cycloalkyl, or heteroaryl;
R¹ is hydrogen or alkyl;
R² is hydrogen, alkyl optionally substituted with —C(O)OR¹, or aryl optionally substituted with alkyl;
R³ is —OC(O)R¹, —C(O)OR′, or —C(O)NR²R⁴;
R⁴ is alkyl optionally substituted with hydroxy; and
R⁵ is selected from the group consisting of hydrogen, hydroxyalkyl, —OC(O)R¹, —C(O)OR¹, —NR′C(O)R², and —NR ‘S(O)₂R².

Clause 3. The compound of clause 1, or a pharmaceutically acceptable salt thereof, having a structure of Formula I(a’):

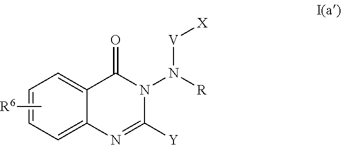

wherein
X is alkyl, aryl, heteroaryl, C₁₋₄ alkylenearyl, or cycloalkyl, wherein X is optionally substituted with one or more substituents selected from the group consisting of alkyl, haloalkyl, alkoxy, halogen, cyano, deuterium, and nitro;
Y is alkyl, cycloalkyl, aryl, C₁₋₄ alkylenearyl, or heteroaryl, wherein Y is optionally substituted with one or more substituents selected from the group consisting of halogen, alkoxy optionally substituted with one or more halogen, alkyl, haloalkyl, and nitro;
R is hydrogen, alkyl, aryl, cycloalkyl, or heteroaryl; and
R⁶ is an optional substituent selected from the group consisting of halogen and alkyl.

Clause 4. The compound of clause 2, or a pharmaceutically acceptable salt thereof, having a structure of Formula I(a):

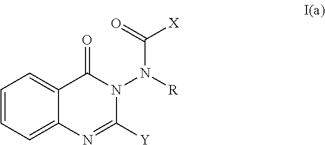

wherein
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro; and
R is hydrogen, alkyl, aryl, cycloalkyl, or heteroaryl.

Clause 5. The compound of clause 3, or a pharmaceutically acceptable salt thereof, wherein
X is alkyl, cycloalkyl, phenyl, naphthyl, furanyl, pyridazyl, benzyl, pyridyl, or thionyl, wherein the cycloalkyl, phenyl, naphthyl, furanyl, pyridazyl, benzyl, pyridyl, or thionyl is optionally substituted with one or more substituents selected from the group consisting of alkoxy, alkyl, haloalkyl, cyano, deuterium, and halogen; and
Y is alkyl, cycloalkyl furanyl, benzyl, thionyl, or phenyl, wherein the cycloalkyl, furanyl, benzyl, thionyl, or phenyl is optionally substituted with one or more groups selected from the group consisting of halogen, haloalkyl, alkoxy optionally substituted with one or more halogen, and nitro.

Clause 6. The compound of clause 4, or a pharmaceutically acceptable salt thereof, wherein X is pyridyl, or phenyl optionally substituted with one or more substituents selected from the group consisting of alkoxy, alkyl, and halogen; and Y is phenyl optionally substituted with one or more groups selected from the group consisting of halogen and nitro.

Clause 7. The compound of any one of clauses 1, 3, and 5, or a pharmaceutically acceptable salt thereof, wherein X is phenyl substituted with 1, 2, 3, 4 or 5 deuterium.

Clause 8. The compound of any one of clauses 1-7, or a pharmaceutically acceptable salt thereof, wherein X is phenyl, phenyl mono-substituted at 2- or 3-position, or phenyl bi-substituted at both 2- and 3-position.

Clause 9. The compound of any one of clauses 1-8, or a pharmaceutically acceptable salt thereof, wherein Y is optionally substituted phenyl.

Clause 10. The compound of clause 9, or a pharmaceutically acceptable salt thereof, wherein Y is 3-chlorophenyl.

Clause 11. The compound of any one of clauses 1 and 2, which is selected from the group consisting of
2-methoxy-N-(2-(3-nitrophenyl)-4-oxoquinazolin-3(4H)-yl)benzamide:
N-(2-(3-chlorophenyl)-4-oxoquinazolin-3(4H)-yl)-2-methoxybenzamide;
N-(2-(3-chlorophenyl)-4-oxoquinazolin-3(4H)-yl)-2-methylbenzamide:
N-(2-(3-chlorophenyl)-4-oxoquinazolin-3(4H)-yl)-2-fluorobenzamide:
N-(2-(3-chlorophenyl)-4-oxoquinazolin-3(4H)-yl) isonicotinamide;
3-chloro-N-(2-(3-chlorophenyl)-4-oxoquinazolin-3(4H)-yl)benzamide:
N-(2-(3-chlorophenyl)-4-oxoquinazolin-3(4H)-yl)-4-fluorobenzamide;
5-bromo-2-chloro-N-(2-(3-chlorophenyl)-4-oxoquinazolin-3(4H)-yl)benzamide;
2-bromo-N-(2-(3-chlorophenyl)-4-oxoquinazolin-3(4H)-yl)-5-methoxybenzamide:
N-(2-(3-chlorophenyl)-4-oxoquinazolin-3(4H)-yl)benzamide:
N-(2-(3-chlorophenyl)-4-oxoquinazolin-3(4H)-yl)benzamide-2,3,4,5,6-d₅;
N-(2-(3-chlorophenyl)-4-oxoquinazolin-3(4H)-yl)-2-(trifluoromethyl)benzamide;
N-(2-(3-chlorophenyl)-4-oxoquinazolin-3(4H)-yl)-2-nitrobenzamide:
2-chloro-N-(2-(3-chlorophenyl)-4-oxoquinazolin-3(4H)-yl)benzamide:
N-(2-(3-chlorophenyl)-4-oxoquinazolin-3(4H)-yl)-3-fluorobenzamide:
N-(2-(3-chlorophenyl)-4-oxoquinazolin-3(4H)-yl)-3-nitrobenzamide:
N-(2-(3-chlorophenyl)-4-oxoquinazolin-3(4H)-yl)-3-methylbenzamide;
N-(2-(3-chlorophenyl)-4-oxoquinazolin-3(4H)-yl)-3-methoxybenzamide:
N-(2-(3-chlorophenyl)-4-oxoquinazolin-3(4H)-yl)-2,6-difluorobenzamide:
N-(2-(3-chlorophenyl)-4-oxoquinazolin-3(4H)-yl)-4-methylbenzamide;
N-(2-(3-chlorophenyl)-4-oxoquinazolin-3(4H)-yl)-4-nitrobenzamide;
N-(2-(3-chlorophenyl)-4-oxoquinazolin-3(4H)-yl)-4-methoxybenzamide:
N-(2-(3-chlorophenyl)-4-oxoquinazolin-3(4H)-yl)-2-naphthamide:
N-(2-(3-chlorophenyl)-4-oxoquinazolin-3(4H)-yl) furan-2-carboxamide;
N-(2-(3-chlorophenyl)-4-oxoquinazolin-3(4H)-yl)-1-naphthamide;
N-(2-(3-chlorophenyl)-4-oxoquinazolin-3(4H)-yl)-2-fluoronicotinamide:
N-(2-(3-chlorophenyl)-4-oxoquinazolin-3(4H)-yl)thiophene-2-carboxamide:
N-(2-(3-chlorophenyl)-4-oxoquinazolin-3(4H)-yl)thiophene-3-carboxamide:
N-(2-(3-chlorophenyl)-4-oxoquinazolin-3(4H)-yl)cyclohexanecarboxamide:
N-(2-(3-chlorophenyl)-4-oxoquinazolin-3(4H)-yl)-2-phenylacetamide:
N-(2-(3-chlorophenyl)-4-oxoquinazolin-3(4H)-yl) acetamide:
N-(2-(3-chlorophenyl)-4-oxoquinazolin-3(4H)-yl)-N-methylbenzamide:
N-(4-oxo-2-(2-(trifluoromethyl)phenyl)quinazolin-3(4H)-yl)benzamide:
N-(2-(3-methoxyphenyl)-4-oxoquinazolin-3(4H)-yl)benzamide:
N-(4-oxo-2-(m-tolyl)quinazolin-3(4H)-yl)benzamide:
N-(2-(3-nitrophenyl)-4-oxoquinazolin-3(4H)-yl)benzamide;
N-(4-oxo-2-(3-(trifluoromethoxy)phenyl)quinazolin-3(4H)-yl)benzamide:
N-(4-oxo-2-(3-(trifluoromethyl)phenyl)quinazolin-3(4H)-yl)benzamide:
N-(2-(4-methoxyphenyl)-4-oxoquinazolin-3(4H)-yl)benzamide:
N-(2-(4-chlorophenyl)-4-oxoquinazolin-3(4H)-yl)benzamide:
2-fluoro-N-(2-methyl-4-oxoquinazolin-3(4H)-yl)benzamide:
N-(4-oxo-2-phenylquinazolin-3(4H)-yl)benzamide;
N-(2-cyclohexyl-4-oxoquinazolin-3(4H)-yl)benzamide:
N-(4-oxo-2-(thiophen-2-yl)quinazolin-3(4H)-yl)benzamide:
N-(4-oxo-2-(thiophen-3-yl)quinazolin-3(4H)-yl)benzamide:
N-(2-(furan-2-yl)-4-oxoquinazolin-3(4H)-yl)benzamide:
N-(2-(3-chlorobenzyl)-4-oxoquinazolin-3(4H)-yl)benzamide:
N-(2-(3-chlorophenyl)-5-methyl-4-oxoquinazolin-3(4H)-yl)benzamide:
N-(5-chloro-2-(3-chlorophenyl)-4-oxoquinazolin-3(4H)-yl)benzamide;
N-(2-(3-chlorophenyl)-6-methyl-4-oxoquinazolin-3(4H)-yl)benzamide:
N-(6-chloro-2-(3-chlorophenyl)-4-oxoquinazolin-3(4H)-yl)benzamide;
N-(7-chloro-2-(3-chlorophenyl)-4-oxoquinazolin-3(4H)-yl)benzamide:
N-(2-(3-chlorophenyl)-8-methyl-4-oxoquinazolin-3(4H)-yl)benzamide:
N-(8-chloro-2-(3-chlorophenyl)-4-oxoquinazolin-3(4H)-yl)benzamide:
3-methyl-N-(4-oxo-2-(thiophen-2-yl)quinazolin-3(4H)-yl)benzamide:
2-methoxy-N-(2-(3-nitrophenyl)-4-oxoquinazolin-3(4H)-yl)benzamide:
N-(2-(5-chlorothiophen-3-yl)-4-oxoquinazolin-3(4H)-yl)-2-fluorobenzamide:
N-(2-(3-chlorophenyl)-4-oxoquinazolin-3(4H)-yl) furan-2-carboxamide:
N-(2-(3-chlorophenyl)-4-oxoquinazolin-3(4H)-yl)-2-nitrobenzamide:
2-fluoro-N-(4-oxo-2-phenylquinazolin-3(4H)-yl)benzamide:
N-(2-cyclohexyl-4-oxoquinazolin-3(4H)-yl)-2-fluorobenzamide:
N-(2-(3-chlorophenyl)-4-oxoquinazolin-3(4H)-yl)-3-cyanobenzamide:
N-(2-(3-chlorophenyl)-4-oxoquinazolin-3(4H)-yl)-5-fluorothiophene-2-carboxamide:
N-(2-(3-chlorophenyl)-4-oxoquinazolin-3(4H)-yl)-4-cyanobenzamide:
N-(2-(3-chlorophenyl)-4-oxoquinazolin-3(4H)-yl) nicotinamide:
N-(2-(3-chlorophenyl)-4-oxoquinazolin-3(4H)-yl) picolinamide:
N-(2-(3-chlorophenyl)-4-oxoquinazolin-3(4H)-yl)cyclohexanesulfonamide:
N-(2-(3-chlorophenyl)-6-methyl-4-oxoquinazolin-3(4H)-yl)-2-fluorobenzamide:
N-(2-(3-chlorophenyl)-4-oxoquinazolin-3(4H)-yl)pyridazine-3-carboxamide:
2-fluoro-N-(2-methyl-4-oxoquinazolin-3(4H)-yl)benzamide:
N-(6-chloro-2-(3-chlorophenyl)-4-oxoquinazolin-3(4H)-yl)-2-fluorobenzamide;
2-bromo-N-(2-(3-chlorophenyl)-4-oxoquinazolin-3(4H)-yl)benzamide:
N-(2-(5-chlorothiophen-3-yl)-4-oxoquinazolin-3(4H)-yl)-2-fluorobenzamide:
N-(2-(3-chlorophenyl)-4-oxoquinazolin-3(4H)-yl)-5-fluorothiophene-2-carboxamide:
4-chloro-N-(2-(3-chlorophenyl)-4-oxoquinazolin-3(4H)-yl)benzenesulfonamide; and
N-(2-(3-chlorophenyl)-4-oxoquinazolin-3(4H)-yl)-2-fluorobenzenesulfonamide,
   or a pharmaceutically acceptable salt thereof.

Clause 12. The compound of clause 11, which is
N-(2-(3-chlorophenyl)-4-oxoquinazolin-3(4H)-yl)benzamide; or
N-(2-(3-chlorophenyl)-4-oxoquinazolin-3(4H)-yl)benzamide-2,3,4,5,6-d₅,
   or a pharmaceutically acceptable salt thereof.

Clause 13. The compound of clause 2, or a pharmaceutically acceptable salt thereof, wherein the compound has a structure of Formula I (b):

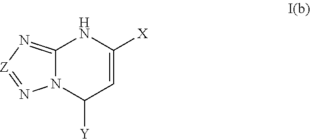

wherein
Z is N or CR⁵.
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
R⁵ is hydrogen, hydroxyalkyl, —OC(O)R¹, —C(O)OR¹, —NR¹C(O)R², or —NR¹S(O)₂R²;
R¹ is hydrogen or alkyl; and
R² is hydrogen, alkyl optionally substituted with —C(O)OR¹, or aryl optionally substituted with alkyl.

Clause 14. The compound of clause 13, or a pharmaceutically acceptable salt thereof, wherein
X is phenyl optionally substituted with one or more substituents selected from the group consisting of halogen, alkoxy, and alkyl,
or X is thiophenyl; and
Y is phenyl or thiophenyl, that is optionally substituted with one or more substituents selected from the group consisting of alkyl, halogen, alkoxy, and alkoxy substituted with one or more halogen,
or Y is 1,3-benzodioxolyl.

Clause 15. The compound of clause 13, wherein the compound is selected from the group consisting of

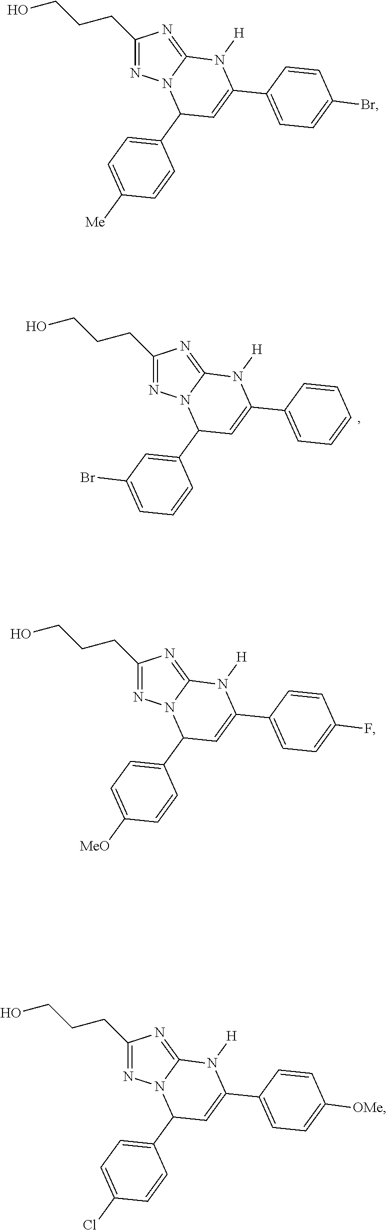 [SMILES: Cc1ccc(C2C=C(c3ccc(Br)cc3)Nc3nc(CCCO)nn32)cc1 ; OCCCc1nc2n(n1)C(c1cccc(Br)c1)C=C(c1ccccc1)N2 ; COc1ccc(C2C=C(c3ccc(F)cc3)Nc3nc(CCCO)nn32)cc1 ; COc1ccc(C2=CC(c3ccc(Cl)cc3)n3nc(CCCO)nc3N2)cc1]

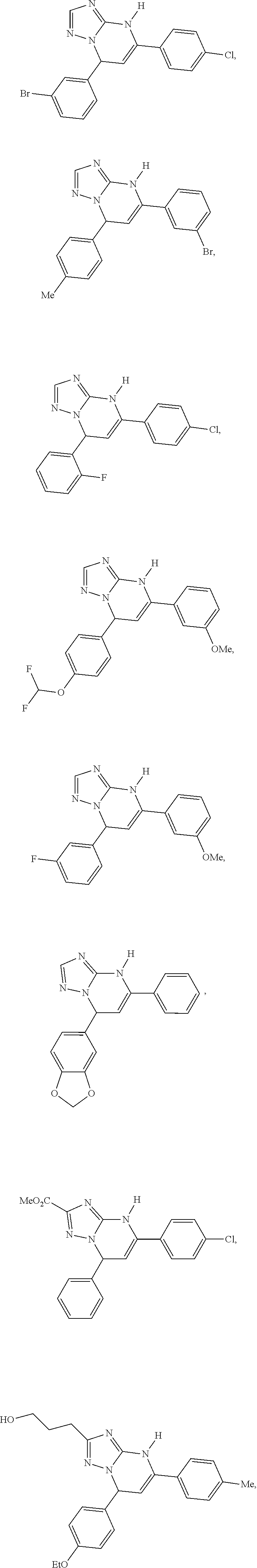

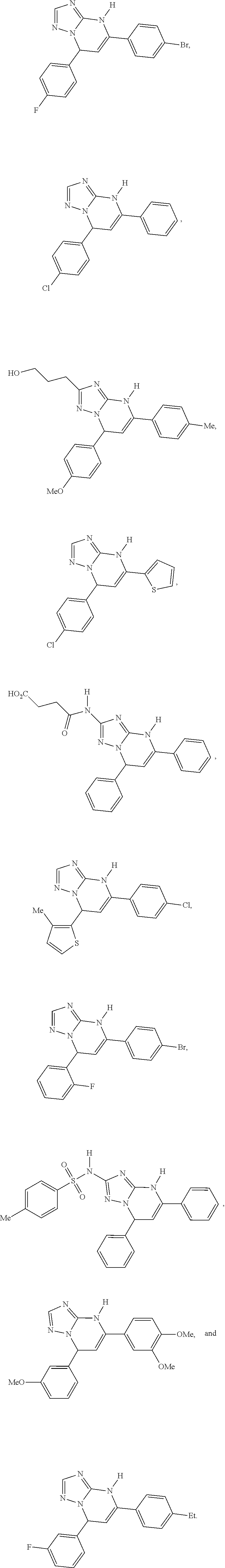

Clause 16. The compound of clause 2, or a pharmaceutically acceptable salt thereof, wherein the compound has a structure of Formula I (c):

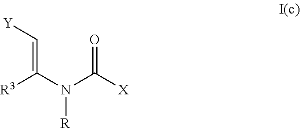

wherein
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
R is hydrogen, alkyl, aryl, cycloalkyl, or heteroaryl;
R³ is —OC(O)R¹, —C(O)OR¹, or —C(O)NR²R⁴;
R¹ is hydrogen or alkyl;
R² is hydrogen, alkyl optionally substituted with —C(O)OR¹, or aryl optionally substituted with alkyl; and
R⁴ is alkyl optionally substituted with hydroxy.

Clause 17. The compound of clause 16, or a pharmaceutically acceptable salt thereof, wherein X is phenyl optionally substituted with alkoxy;
Y is phenyl optionally substituted with halogen or nitro;
R is hydrogen;
R³ is —OC(O)R¹, —C(O)OR¹, or —C(O)NHR⁴;
R¹ is alkyl;
R⁴ is alkyl substituted with hydroxy.

Clause 18. The compound of clause 16, wherein the compound is selected from the group consisting of

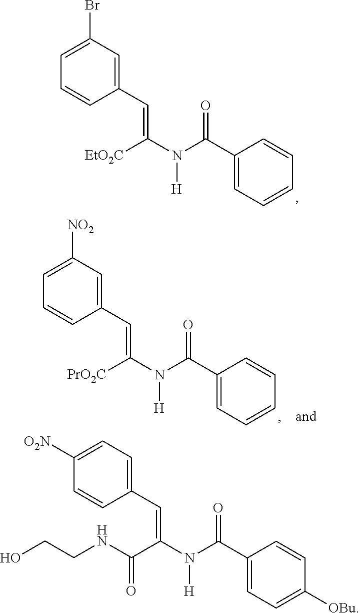

Clause 19. The compound of clause 2, or a pharmaceutically acceptable salt thereof, wherein the compound has a structure of Formula I (d):

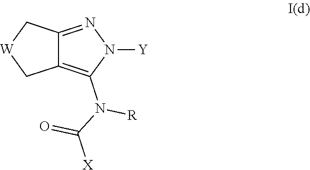

wherein
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
W is S, S(O), or S(O)₂; and R is hydrogen, alkyl, aryl, cycloalkyl, or heteroaryl.

Clause 20. The compound of clause 19, or a pharmaceutically acceptable salt thereof, wherein
W is S or S(O)₂;
X is alkyl;
Y is phenyl optionally substituted by halogen; and
R is hydrogen.

Clause 21. The compound of clause 19, wherein the compound is

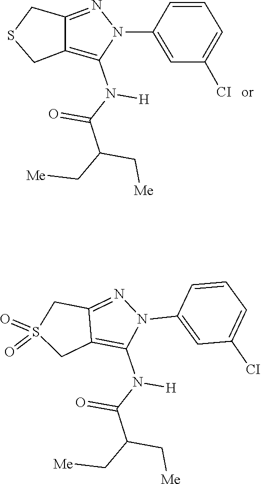

Clause 22. A composition comprising the compound of any of clauses 1-21, or a pharmaceutically acceptable salt thereof, and a pharmaceutically acceptable carrier.

Clause 23. A method of treating a parasitic infection in a subject in need thereof, the method comprises administering to the subject an effective amount of the compound of any of clauses 1-21, or a pharmaceutically acceptable salt thereof, or the composition of clause 22.

Clause 24. The method of clause 23, wherein the parasitic infection is a parasitic flatworm infection.

Clause 25. The method of clause 24, wherein the parasitic flatworm is trematode, cestode, monogenean, schistosome, tapeworm, or a combination thereof.

Clause 26. A method of treating schistosomiasis in a subject in need thereof, the method comprises administering to the subject an effective amount of the compound of any of clauses 1-21, or a pharmaceutically acceptable salt thereof, or the composition of clause 22.

Clause 27. A method of activating a transient receptor potential channel of a parasitic flatworm, the method comprising introducing to the parasitic flatworm an effective amount of the compound of any of clauses 1-21, or a salt thereof, or the composition of clause 22.

Clause 28. A method of causing paralysis or inhibiting growth of a parasitic flatworm, the method comprising introducing to the parasitic flatworm an effective amount of the compound of any of clauses 1-21, or a salt thereof, or the composition of clause 22.

Clause 29. The method of any one of clauses 27-28, wherein the parasitic flatworm is in an infected subject.

Clause 30. The method of any one of clauses 23-26 and 29, wherein the subject is a human.

We claim:

1. A compound of Formula (I′), or a pharmaceutically acceptable salt thereof: 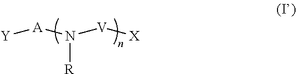
wherein
A is a ring system selected from the group consisting of 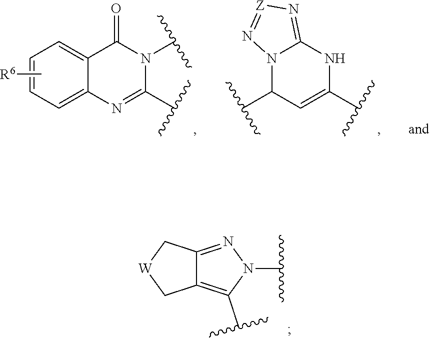
or A is an alkenyl of 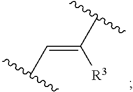
X is alkyl, aryl, heteroaryl, C₁₋₄ alkylenearyl, or cycloalkyl, wherein X is optionally substituted with one or more substituents selected from the group consisting of alkyl, haloalkyl, alkoxy, halogen, cyano, deuterium, and nitro;
Y is alkyl, cycloalkyl, aryl, C₁₋₄ alkylenearyl, or heteroaryl, wherein Y is optionally substituted with one or more substituents selected from the group consisting of halogen, alkoxy optionally substituted with one or more halogen, alkyl, haloalkyl, and nitro;
Z is N or CR⁵;
W is S, S(O), or S(O)₂;
V is C(O) or S(O)₂;
n is 0 or 1; and
wherein
R is hydrogen, alkyl, aryl, cycloalkyl, or heteroaryl;
R¹ is hydrogen or alkyl;
R² is hydrogen, alkyl optionally substituted with —C(O)OR¹, or aryl optionally substituted with alkyl;
R³ is —OC(O)R¹, —C(O)OR¹, or —C(O)NR²R⁴,
R⁴ is alkyl optionally substituted with hydroxy;
R⁵ is selected from the group consisting of hydrogen, hydroxyalkyl, —OC(O)R¹, —C(O)OR¹, —NR¹C(O)R², and —NR¹S(O)₂R²; and
R⁶ is an optional substituent selected from the group consisting of halogen, alkyl, haloalkyl, alkoxy, cyano, and nitro,
provided that the compound is not 4-bromo-N-(2-(4-fluorophenyl)-4-oxoquinazolin-3(4H)-yl)benzamide or 4-bromo-N-(2-(2-fluorophenyl)-4-oxoquinazolin-3(4H)-yl)benzamide.

2. The compound of claim 1, or a pharmaceutically acceptable salt thereof, having a structure of Formula (I), 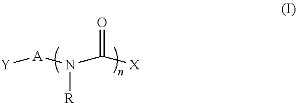
wherein
A is a ring system selected from the group consisting of 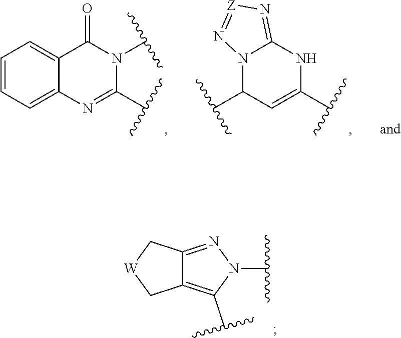
or A is an alkenyl of 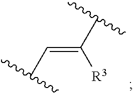
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
Z is N or CR⁵;
W is S, S(O), or S(O)₂;
n is 0 or 1; and
wherein
R is hydrogen, alkyl, aryl, cycloalkyl, or heteroaryl;
R¹ is hydrogen or alkyl;
R² is hydrogen, alkyl optionally substituted with —C(O)OR¹, or aryl optionally substituted with alkyl;
R³ is —OC(O)R¹, —C(O)OR¹, or —C(O)NR²R⁴,
R⁴ is alkyl optionally substituted with hydroxy; and
R⁵ is selected from the group consisting of hydrogen, hydroxyalkyl, —OC(O)R¹, —C(O)OR¹, —NR¹C(O)R², and —NR¹S(O)₂R².

3. The compound of claim 1, or a pharmaceutically acceptable salt thereof, having a structure of Formula I(a′): 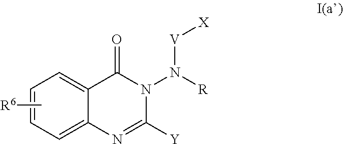
wherein
X is alkyl, aryl, heteroaryl, C₁₋₄ alkylenearyl, or cycloalkyl, wherein X is optionally substituted with one or more substituents selected from the group consisting of alkyl, haloalkyl, alkoxy, halogen, cyano, deuterium, and nitro;
Y is alkyl, cycloalkyl, aryl, C₁₋₄ alkylenearyl, or heteroaryl, wherein Y is optionally substituted with one or more substituents selected from the group consisting of halogen, alkoxy optionally substituted with one or more halogen, alkyl, haloalkyl, and nitro;
R is hydrogen, alkyl, aryl, cycloalkyl, or heteroaryl; and
R⁶ is an optional substituent selected from the group consisting of halogen and alkyl.

4. The compound of claim 2, or a pharmaceutically acceptable salt thereof, having a structure of Formula I(a): 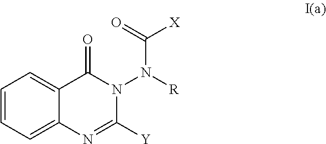
wherein
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro; and
R is hydrogen, alkyl, aryl, cycloalkyl, or heteroaryl.

5. (canceled)

6. The compound of claim 4, or a pharmaceutically acceptable salt thereof, wherein X is pyridyl, or phenyl optionally substituted with one or more substituents selected from the group consisting of alkoxy, alkyl, and halogen; and
Y is phenyl optionally substituted with one or more groups selected from the group consisting of halogen and nitro.

7. The compound of claim 1, or a pharmaceutically acceptable salt thereof, wherein
X is phenyl substituted with 1, 2, 3, 4 or 5 deuterium, or
X is phenyl mono-substituted at 2- or 3-position, or phenyl bi-substituted at both 2- and 3-position.

8. (canceled)

9. The compound of claim 1, or a pharmaceutically acceptable salt thereof, wherein Y is optionally substituted phenyl.

10. The compound of claim 9, or a pharmaceutically acceptable salt thereof, wherein Y is 3-chlorophenyl.

11. The compound of claim 1, which is selected from the group consisting of
2-methoxy-N-(2-(3-nitrophenyl)-4-oxoquinazolin-3(4H)-yl)benzamide;
N-(2-(3-chlorophenyl)-4-oxoquinazolin-3(4H)-yl)-2-methoxybenzamide;
N-(2-(3-chlorophenyl)-4-oxoquinazolin-3(4H)-yl)-2-methylbenzamide;
N-(2-(3-chlorophenyl)-4-oxoquinazolin-3(4H)-yl)-2-fluorobenzamide;
N-(2-(3-chlorophenyl)-4-oxoquinazolin-3(4H)-yl) isonicotinamide;
3-chloro-N-(2-(3-chlorophenyl)-4-oxoquinazolin-3(4H)-yl)benzamide;
N-(2-(3-chlorophenyl)-4-oxoquinazolin-3(4H)-yl)-4-fluorobenzamide;
5-bromo-2-chloro-N-(2-(3-chlorophenyl)-4-oxoquinazolin-3(4H)-yl)benzamide;
2-bromo-N-(2-(3-chlorophenyl)-4-oxoquinazolin-3(4H)-yl)-5-methoxybenzamide;
N-(2-(3-chlorophenyl)-4-oxoquinazolin-3(4H)-yl)benzamide;
N-(2-(3-chlorophenyl)-4-oxoquinazolin-3(4H)-yl)benzamide-2,3,4,5,6-d₅;
N-(2-(3-chlorophenyl)-4-oxoquinazolin-3(4H)-yl)-2-(trifluoromethyl)benzamide;
N-(2-(3-chlorophenyl)-4-oxoquinazolin-3(4H)-yl)-2-nitrobenzamide;
2-chloro-N-(2-(3-chlorophenyl)-4-oxoquinazolin-3(4H)-yl)benzamide;
N-(2-(3-chlorophenyl)-4-oxoquinazolin-3(4H)-yl)-3-fluorobenzamide;
N-(2-(3-chlorophenyl)-4-oxoquinazolin-3(4H)-yl)-3-nitrobenzamide;
N-(2-(3-chlorophenyl)-4-oxoquinazolin-3(4H)-yl)-3-methylbenzamide;
N-(2-(3-chlorophenyl)-4-oxoquinazolin-3(4H)-yl)-3-methoxybenzamide;
N-(2-(3-chlorophenyl)-4-oxoquinazolin-3(4H)-yl)-2,6-difluorobenzamide;
N-(2-(3-chlorophenyl)-4-oxoquinazolin-3(4H)-yl)-4-methylbenzamide;
N-(2-(3-chlorophenyl)-4-oxoquinazolin-3(4H)-yl)-4-nitrobenzamide;
N-(2-(3-chlorophenyl)-4-oxoquinazolin-3(4H)-yl)-4-methoxybenzamide;
N-(2-(3-chlorophenyl)-4-oxoquinazolin-3(4H)-yl)-2-naphthamide;
N-(2-(3-chlorophenyl)-4-oxoquinazolin-3(4H)-yl) furan-2-carboxamide;
N-(2-(3-chlorophenyl)-4-oxoquinazolin-3(4H)-yl)-1-naphthamide;
N-(2-(3-chlorophenyl)-4-oxoquinazolin-3(4H)-yl)-2-fluoronicotinamide;
N-(2-(3-chlorophenyl)-4-oxoquinazolin-3(4H)-yl)thiophene-2-carboxamide;
N-(2-(3-chlorophenyl)-4-oxoquinazolin-3(4H)-yl)thiophene-3-carboxamide;
N-(2-(3-chlorophenyl)-4-oxoquinazolin-3(4H)-yl)cyclohexanecarboxamide;
N-(2-(3-chlorophenyl)-4-oxoquinazolin-3(4H)-yl)-2-phenylacetamide;
N-(2-(3-chlorophenyl)-4-oxoquinazolin-3(4H)-yl) acetamide;
N-(2-(3-chlorophenyl)-4-oxoquinazolin-3(4H)-yl)-N-methylbenzamide;
N-(4-oxo-2-(2-(trifluoromethyl)phenyl)quinazolin-3(4H)-yl)benzamide;
N-(2-(3-methoxyphenyl)-4-oxoquinazolin-3(4H)-yl)benzamide;
N-(4-oxo-2-(m-tolyl)quinazolin-3(4H)-yl)benzamide;
N-(2-(3-nitrophenyl)-4-oxoquinazolin-3(4H)-yl)benzamide;
N-(4-oxo-2-(3-(trifluoromethoxy)phenyl)quinazolin-3(4H)-yl)benzamide;
N-(4-oxo-2-(3-(trifluoromethyl)phenyl)quinazolin-3(4H)-yl)benzamide;
N-(2-(4-methoxyphenyl)-4-oxoquinazolin-3(4H)-yl)benzamide;
N-(2-(4-chlorophenyl)-4-oxoquinazolin-3(4H)-yl)benzamide;
2-fluoro-N-(2-methyl-4-oxoquinazolin-3(4H)-yl)benzamide;
N-(4-oxo-2-phenylquinazolin-3(4H)-yl)benzamide;
N-(2-cyclohexyl-4-oxoquinazolin-3(4H)-yl)benzamide;
N-(4-oxo-2-(thiophen-2-yl)quinazolin-3(4H)-yl)benzamide;
N-(4-oxo-2-(thiophen-3-yl)quinazolin-3(4H)-yl)benzamide;
N-(2-(furan-2-yl)-4-oxoquinazolin-3(4H)-yl)benzamide;
N-(2-(3-chlorobenzyl)-4-oxoquinazolin-3(4H)-yl)benzamide;
N-(2-(3-chlorophenyl)-5-methyl-4-oxoquinazolin-3(4H)-yl)benzamide;
N-(5-chloro-2-(3-chlorophenyl)-4-oxoquinazolin-3(4H)-yl)benzamide;
N-(2-(3-chlorophenyl)-6-methyl-4-oxoquinazolin-3(4H)-yl)benzamide;
N-(6-chloro-2-(3-chlorophenyl)-4-oxoquinazolin-3(4H)-yl)benzamide;
N-(7-chloro-2-(3-chlorophenyl)-4-oxoquinazolin-3(4H)-yl)benzamide;
N-(2-(3-chlorophenyl)-8-methyl-4-oxoquinazolin-3(4H)-yl)benzamide;
N-(8-chloro-2-(3-chlorophenyl)-4-oxoquinazolin-3(4H)-yl)benzamide;
3-methyl-N-(4-oxo-2-(thiophen-2-yl)quinazolin-3(4H)-yl)benzamide;
2-methoxy-N-(2-(3-nitrophenyl)-4-oxoquinazolin-3(4H)-yl)benzamide;
N-(2-(5-chlorothiophen-3-yl)-4-oxoquinazolin-3(4H)-yl)-2-fluorobenzamide;
N-(2-(3-chlorophenyl)-4-oxoquinazolin-3(4H)-yl) furan-2-carboxamide;
N-(2-(3-chlorophenyl)-4-oxoquinazolin-3(4H)-yl)-2-nitrobenzamide;
2-fluoro-N-(4-oxo-2-phenylquinazolin-3(4H)-yl)benzamide;
N-(2-cyclohexyl-4-oxoquinazolin-3(4H)-yl)-2-fluorobenzamide;
N-(2-(3-chlorophenyl)-4-oxoquinazolin-3(4H)-yl)-3-cyanobenzamide;
N-(2-(3-chlorophenyl)-4-oxoquinazolin-3(4H)-yl)-5-fluorothiophene-2-carboxamide;
N-(2-(3-chlorophenyl)-4-oxoquinazolin-3(4H)-yl)-4-cyanobenzamide;
N-(2-(3-chlorophenyl)-4-oxoquinazolin-3(4H)-yl) nicotinamide;
N-(2-(3-chlorophenyl)-4-oxoquinazolin-3(4H)-yl) picolinamide;
N-(2-(3-chlorophenyl)-4-oxoquinazolin-3(4H)-yl)cyclohexanesulfonamide;
N-(2-(3-chlorophenyl)-6-methyl-4-oxoquinazolin-3(4H)-yl)-2-fluorobenzamide;
N-(2-(3-chlorophenyl)-4-oxoquinazolin-3(4H)-yl)pyridazine-3-carboxamide;
2-fluoro-N-(2-methyl-4-oxoquinazolin-3(4H)-yl)benzamide;
N-(6-chloro-2-(3-chlorophenyl)-4-oxoquinazolin-3(4H)-yl)-2-fluorobenzamide;
2-bromo-N-(2-(3-chlorophenyl)-4-oxoquinazolin-3(4H)-yl)benzamide;
N-(2-(5-chlorothiophen-3-yl)-4-oxoquinazolin-3(4H)-yl)-2-fluorobenzamide;
N-(2-(3-chlorophenyl)-4-oxoquinazolin-3(4H)-yl)-5-fluorothiophene-2-carboxamide;
4-chloro-N-(2-(3-chlorophenyl)-4-oxoquinazolin-3(4H)-yl)benzenesulfonamide; and
N-(2-(3-chlorophenyl)-4-oxoquinazolin-3(4H)-yl)-2-fluorobenzenesulfonamide,
or a pharmaceutically acceptable salt thereof.

12. (canceled)

13. The compound of claim 2, or a pharmaceutically acceptable salt thereof, wherein the compound has a structure of Formula I (b): 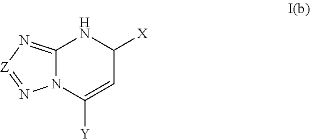
wherein
Z is N or CR⁵;
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
R⁵ is hydrogen, hydroxyalkyl, —OC(O)R¹, —C(O)OR¹, —NR¹C(O)R², or —NR¹S(O)₂R²;
R¹ is hydrogen or alkyl; and
R² is hydrogen, alkyl optionally substituted with —C(O)OR¹, or aryl optionally substituted with alkyl.

14. The compound of claim 13, or a pharmaceutically acceptable salt thereof, wherein
X is phenyl optionally substituted with one or more substituents selected from the group consisting of halogen, alkoxy, and alkyl,
or X is thiophenyl; and
Y is phenyl or thiophenyl, that is optionally substituted with one or more substituents selected from the group consisting of alkyl, halogen, alkoxy, and alkoxy substituted with one or more halogen,
or Y is 1,3-benzodioxolyl.

15. The compound of claim 13, wherein the compound is selected from the group consisting of

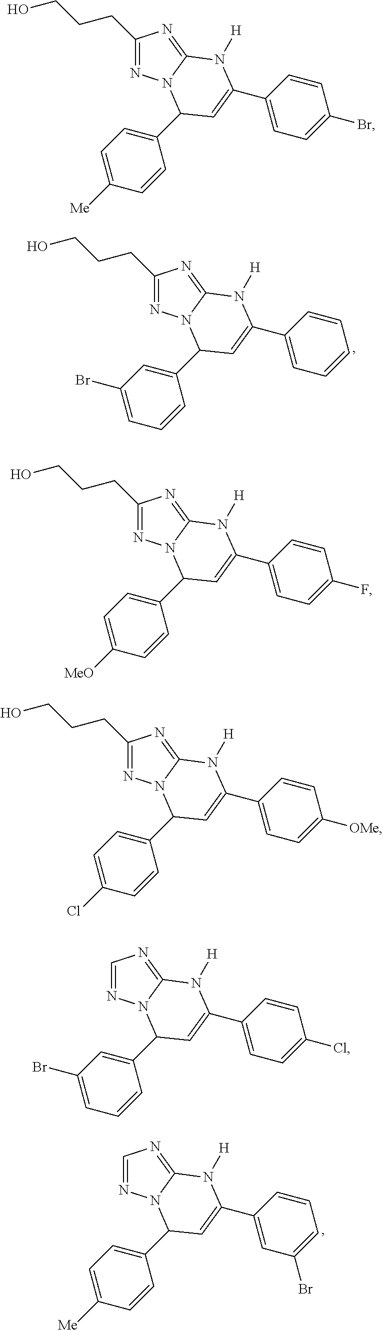

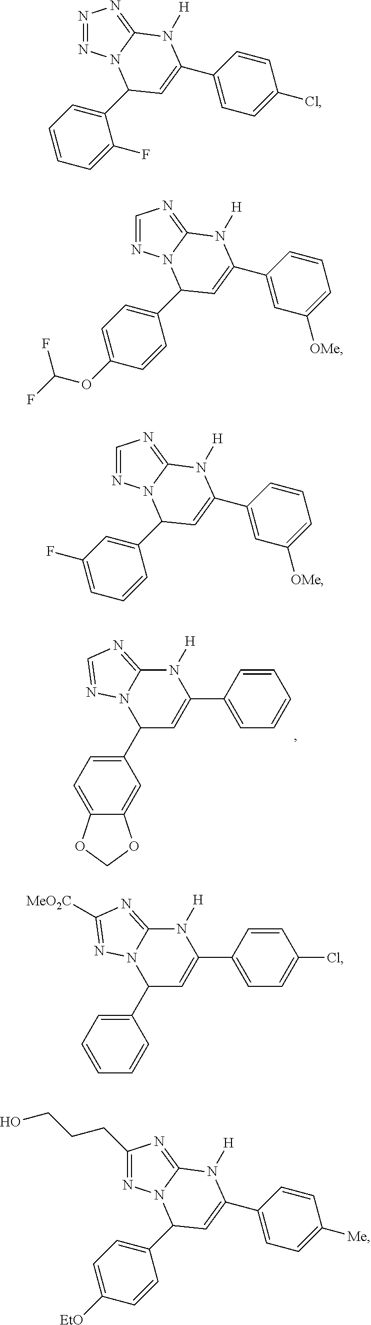

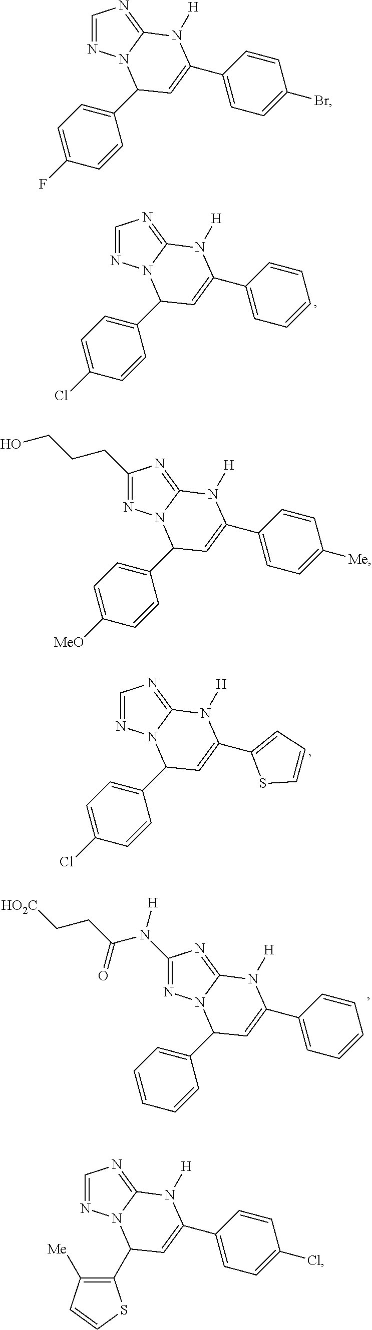

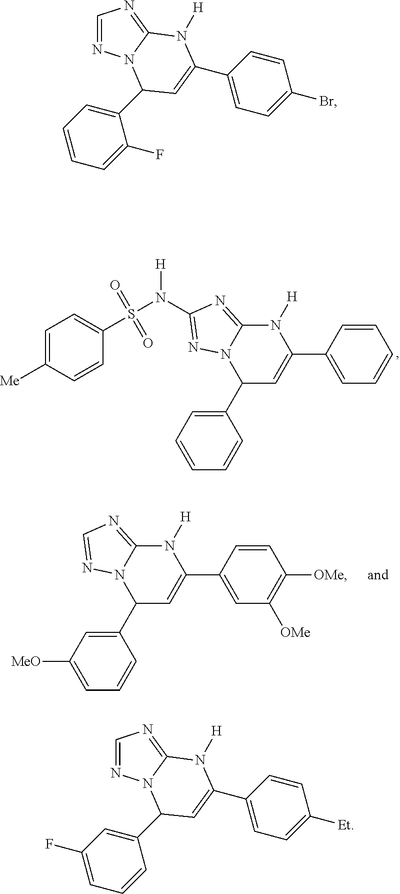 [SMILES: Fc1ccccc1C1C=C(c2ccc(Br)cc2)Nc2ncnn21 ; Cc1ccc(S(=O)(=O)Nc2nc3n(n2)C(c2ccccc2)C=C(c2ccccc2)N3)cc1 ; COc1cccc(C2C=C(c3ccc(OC)c(OC)c3)Nc3ncnn32)c1 ; CCc1ccc(C2=CC(c3cccc(F)c3)n3ncnc3N2)cc1]

16. The compound of claim 2, or a pharmaceutically acceptable salt thereof, wherein the compound has a structure of Formula I (c): 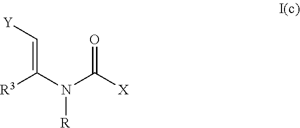
wherein
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
R is hydrogen, alkyl, aryl, cycloalkyl, or heteroaryl;
R³ is —OC(O)R¹, —C(O)OR¹, or —C(O)NR²R⁴;
R¹ is hydrogen or alkyl;
R² is hydrogen, alkyl optionally substituted with —C(O)OR¹, or aryl optionally substituted with alkyl; and
R⁴ is alkyl optionally substituted with hydroxy.

17. The compound of claim 16, or a pharmaceutically acceptable salt thereof, wherein X is phenyl optionally substituted with alkoxy;
Y is phenyl optionally substituted with halogen or nitro;
R is hydrogen;
R³ is —OC(O)R¹, —C(O)OR¹, or —C(O)NHR⁴;
R¹ is alkyl;
R⁴ is alkyl substituted with hydroxy.

18. The compound of claim 16, wherein the compound is selected from the group consisting of

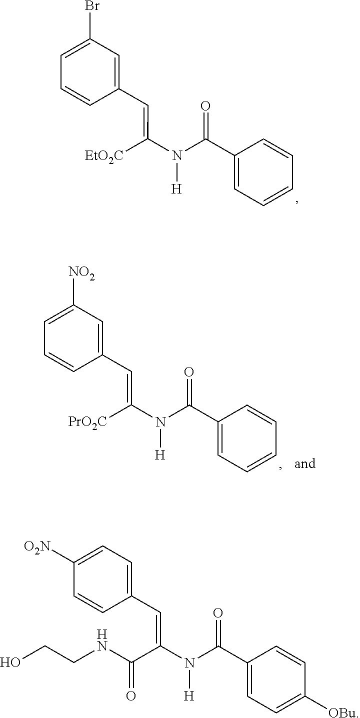

19. The compound of claim 2, or a pharmaceutically acceptable salt thereof, wherein the compound has a structure of Formula I (d): 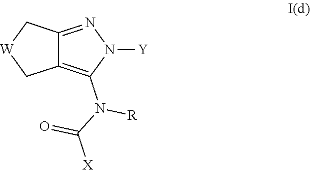
wherein
X is alkyl, aryl, heteroaryl, or cycloalkyl, wherein X is optionally substituted with one or more substituents selected from the group consisting of alkyl, haloalkyl, alkoxy, halogen, and nitro;
Y is alkyl, cycloalkyl, aryl, or heteroaryl, wherein Y is optionally substituted with one or more substituents selected from the group consisting of halogen, alkoxy optionally substituted with one or more halogen, alkyl, haloalkyl, and nitro;
W is S, S(O), or S(O)₂; and
R is hydrogen, alkyl, aryl, cycloalkyl, or heteroaryl.

20. The compound of claim 19, or a pharmaceutically acceptable salt thereof, wherein
W is S or S(O)₂;
X is alkyl;
Y is phenyl optionally substituted by halogen; and
R is hydrogen.

21. The compound of claim 19, wherein the compound is

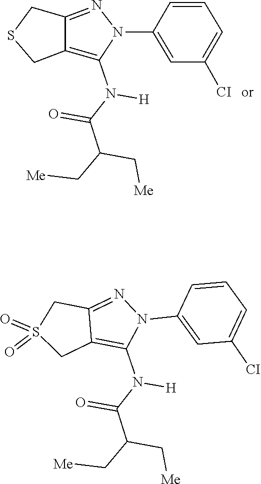

22. A composition comprising the compound of claim 1, or a pharmaceutically acceptable salt thereof, and a pharmaceutically acceptable carrier.

23. A method of treating a parasitic infection in a subject in need thereof, the method comprises administering to the subject an effective amount of the compound of claim 1, or a pharmaceutically acceptable salt thereof.

24. (canceled)

25. (canceled)

26. A method of treating schistosomiasis in a subject in need thereof, the method comprises administering to the subject an effective amount of the compound of claim 1, or a pharmaceutically acceptable salt thereof.

27. A method of activating a transient receptor potential channel of a parasitic flatworm, the method comprising introducing to the parasitic flatworm an effective amount of the compound of claim 1, or a salt thereof.

28. A method of causing paralysis or inhibiting growth of a parasitic flatworm, the method comprising introducing to the parasitic flatworm an effective amount of the compound of claim 1, or a salt thereof.

29. (canceled)

30. (canceled)
